SEQUENCE LISTING (59 sequences)

SEQ ID NO: 1 - protein, 7 aa, synthetic construct
  REGION 1..7: note = Synthetic Construct
SSISSNY

SEQ ID NO: 2 - skipped

SEQ ID NO: 3 - protein, 18 aa, synthetic construct
  REGION 1..18: note = Synthetic Construct
QQGSSIPRTRSEGAPSWK

SEQ ID NO: 4 - protein, 8 aa, synthetic construct
  REGION 1..8: note = Synthetic Construct
GYTFTSYW

SEQ ID NO: 5 - protein, 8 aa, synthetic construct
  REGION 1..8: note = Synthetic Construct
IGPNSGGT

SEQ ID NO: 6 - protein, 22 aa, synthetic construct
  REGION 1..22: note = Synthetic Construct
ARRMVKGCYGLLGPRDHGHRLL

SEQ ID NO: 7 - protein, 11 aa, synthetic construct
  REGION 1..11: note = Synthetic Construct
QSIVHSNGNTY

SEQ ID NO: 8 - skipped

SEQ ID NO: 9 - protein, 9 aa, synthetic construct
  REGION 1..9: note = Synthetic Construct
FQGSHVPYT

SEQ ID NO: 10 - protein, 8 aa, synthetic construct
  REGION 1..8: note = Synthetic Construct
GYTFTDYY

SEQ ID NO: 11 - protein, 8 aa, synthetic construct
  REGION 1..8: note = Synthetic Construct
INPNNGGT

SEQ ID NO: 12 - protein, 11 aa, synthetic construct
  REGION 1..11: note = Synthetic Construct
ARYDYAWYFDV

SEQ ID NO: 13 - protein, 107 aa, synthetic construct
  REGION 1..107: note = Synthetic Construct
DIELTQSPTTMAASPGEKITITCSASSSISSNYLHWYQQKPGFSPKLLIYRTSNLASGVPARFSGSGSGTSYSLTIGTMEAEDVATYYCQQGSSIPRTRSEGAPSWK

SEQ ID NO: 14 - protein, 112 aa, synthetic construct
  REGION 1..112: note = Synthetic Construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 15 - protein, 117 aa, synthetic construct
  REGION 1..117: note = Synthetic Construct
VKLQESGAELVKPGASVKLSCKASGYTFTSYWMHWVKQRPGRGLEWIGRIGPNSGGTKYNEKFKSKATLTVDKPSSTAYMQLSSLTSEDSAVYYCARRMVKGCYGLLGPRDHGHRLL

SEQ ID NO: 16 - protein, 117 aa, synthetic construct
  REGION 1..117: note = Synthetic Construct
VKLQESGPELVKPGASVKISCKASGYTFTDYYMNWVKQSHGKSLEWIGDINPNNGGTSYNQKFKGKATLTVDKSSSTAYMELRSLTSEDSAVYYCARYDYAWYFDVWGQGTTVTVSS

SEQ ID NO: 17 - protein, 272 aa, synthetic construct
  REGION 1..272: note = Synthetic Construct
HHHHHHVKLQESGAELVKPGASVKLSCKASGYTFTSYWMHWVKQRPGRGLEWIGRIGPNSGGTKYNEKFKSKATLTVDKPSSTAYMQLSSLTSEDSAVYYCARRMVKGCYGLLGPRDHGHRLLKGRIPAHWRPLLVDPSSVPSLASGGGGGSGGGGSWISAEFALDIELTQSPTTMAASPGEKITITCSASSSISSNYLHWYQQKPGFSPKLLIYRTSNLASGVPARFSGSGSGTSYSLTIGTMEAEDVATYYCQQGSSIPRTRSEGAPSWK

SEQ ID NO: 18 - protein, 274 aa, synthetic construct
  REGION 1..274: note = Synthetic Construct
MSVPTQVLGLLLLWLTDARCVKLQESGPELVKPGASVKISCKASGYTFTDYYMNWVKQSHGKSLEWIGDINPNNGGTSYNQKFKGKATLTVDKSSSTAYMELRSLTSEDSAVYYCARYDYAWYFDVWGQGTTVTVSSGGGGSGGGGSGGGGDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKRIEGRHHHHHH

SEQ ID NO: 19 - DNA, 321 bp, synthetic construct
  misc_feature 1..321: note = Synthetic Construct
  source 1..321: mol_type = other DNA
gacattgagctcacccagtctccaaccaccatggctgcatctcccggggagaagatcactatcacctgcagtgccagctcaagtataagttccaattacttgcattggtatcagcagaagccaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtagtagtataccacgtacacgttcggagggggcaccaagctggaaa

SEQ ID NO: 20 - DNA, 338 bp, synthetic construct
  misc_feature 1..338: note = Synthetic Construct
  source 1..338: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaacg

SEQ ID NO: 21 - DNA, 352 bp, synthetic construct
  misc_feature 1..352: note = Synthetic Construct
  source 1..352: mol_type = other DNA
gtgaaactgcaggagtcaggggctgagcttgtgaagcctggggcttcagtgaagctgtcctgcaaggcttctggctacaccttcaccagctactggatgcactgggtgaagcagaggcctggacgaggccttgagtggattggaaggattggtcctaatagtggtggtactaagtacaatgagaagttcaagagcaaggccacactgactgtagacaaaccctccagcacagcctacatgcagctcagcagcctgacatctgaggactctgcggtctattattgtgcaagaagaatggtaaaggggtgctatggactactggggccaagggaccacggtcaccgtctcctca

SEQ ID NO: 22 - DNA, 351 bp, synthetic construct
  misc_feature 1..351: note = Synthetic Construct
  source 1..351: mol_type = other DNA
gtgaagctgcaggagtctggacctgagctggtgaagcctggggcttcagtgaagatatcctgtaaggcttctggatacacgttcactgactactacatgaactgggtgaagcagagccatggaaagagccttgagtggattggagatattaatcctaacaatggtggtactagctacaaccagaagttcaagggcaaggccacattgactgtagacaagtcctccagcacagcctacatggagctccgcagcctgacatctgaggactctgcagtctattactgtgcaagatatgattacgcttggtacttcgatgtctggggccaagggaccacggtcaccgtctcctca

SEQ ID NO: 23 - DNA, 837 bp, synthetic construct
  misc_feature 1..837: note = Synthetic Construct
  source 1..837: mol_type = other DNA
gccgccaccatgagtgtgcccactcaggtcctggggttgctgctgctgtggcttacagatgccagatgtgtgaagctgcaggagtctggacctgagctggtgaagcctggggcttcagtgaagatatcctgtaaggcttctggatacacgttcactgactactacatgaactgggtgaagcagagccatggaaagagccttgagtggattggagatattaatcctaacaatggtggtactagctacaaccagaagttcaagggcaaggccacattgactgtagacaagtcctccagcacagcctacatggagctccgcagcctgacatctgaggactctgcagtctattactgtgcaagatatgattacgcttggtacttcgatgtctggggccaagggaccacggtcaccgtctcctcaggcggaggtgggtcgggtggcggcggatctggcggaggtggggatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaacggatcgaaggccggcatcaccatcatcaccactgatag

SEQ ID NO: 24 - DNA, 825 bp, synthetic construct
  misc_feature 1..825: note = Synthetic Construct
  source 1..825: mol_type = other DNA
atgtccgtgcctacccaggtgctcggactcctgctgctgtggctcaccgacgccaggtgtgtgaagctgcaggagagcggacccgagctggtgaagcctggagcctccgtgaagatcagctgcaaggcttccggatacaccttcaccgactactatatgaactgggtgaagcagagccacggcaagagcctggagtggatcggcgacatcaaccctaacaacggcggcacctcctacaaccagaagttcaagggcaaggctacactgaccgtggacaagtcctccagcaccgcctacatggagctcaggagcctgacctccgaggattccgccgtctattactgtgcccggtacgactacgcctggtatttcgacgtgtggggccagggcacaaccgtcacagtctccagcggaggaggaggaagcggcggcggaggatccggaggcggaggcgatgtcctgatgacacagacacctctgagcctccccgtgagcctgggagaccaagcctccatctcctgcaggtcctcccagtccatcgtgcacagcaatggcaacacctacctggagtggtatctgcagaagcctggccagtcccccaagctgctgatctacaaggtgtccaaccggttcagcggcgtccctgacaggttctccggatccggaagcggcacagatttcaccctgaagatcagcagggtcgaggccgaggacctgggagtgtactactgcttccagggctcccatgtcccttacaccttcggcggcggcaccaaactggagatcaagcggatcgagggcaggcatcaccaccatcaccactga

SEQ ID NO: 25 - protein, 10 aa, synthetic construct
  REGION 1..10: note = Synthetic Construct
KSVSTSGYSY

SEQ ID NO: 26 - skipped

SEQ ID NO: 27 - protein, 16 aa, synthetic construct
  REGION 1..16: note = Synthetic Construct
QHIRELTRSEGGPSWK

SEQ ID NO: 28 - protein, 8 aa, synthetic construct
  REGION 1..8: note = Synthetic Construct
GYTFTSYW

SEQ ID NO: 29 - protein, 8 aa, synthetic construct
  REGION 1..8: note = Synthetic Construct
INPSNGGT

SEQ ID NO: 30 - protein, 12 aa, synthetic construct
  REGION 1..12: note = Synthetic Construct
ARRGIRLRHFDY

SEQ ID NO: 31 - protein, 6 aa, synthetic construct
  REGION 1..6: note = Synthetic Construct
QDVGTA

SEQ ID NO: 32 - skipped

SEQ ID NO: 33 - protein, 9 aa, synthetic construct
  REGION 1..9: note = Synthetic Construct
QQYSSYPLT

SEQ ID NO: 34 - protein, 108 aa, synthetic construct
  REGION 1..108: note = Synthetic Construct
DIVMTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWK

SEQ ID NO: 35 - protein, 107 aa, synthetic construct
  REGION 1..107: note = Synthetic Construct
DIQMTQSPKFMSTSVGDRVSITCKASQDVGTAVAWYQQKPGQSPKLLIYWASTRHTGVPDRFTGSGSGTDFTLTISNVQSEDLADYFCQQYSSYPLTFGAGTKLELK

SEQ ID NO: 36 - protein, 117 aa, synthetic construct
  REGION 1..117: note = Synthetic Construct
VKLQESGTELVKPGASVKLSCKASGYTFTSYWMHWVKQRPGQGLEWIGNINPSNGGTNYNEKFKSKATLTVDKSSSTAYMQLSSLTSEDSAVYYCARRGIRLRHFDYWGQGTTVTVS

SEQ ID NO: 37 - protein, 275 aa, synthetic construct
  REGION 1..275: note = Synthetic Construct
VKLQESGTELVKPGASVKLSCKASGYTFTSYWMHWVKQRPGQGLEWIGNINPSNGGTNYNEKFKSKATLTVDKSSSTAYMQLSSLTSEDSAVYYCARRGIRLRHFDYWGQGTTVTVSSRANSADIHHTGGRSSMHLEGPIRPIVSRISGGGGGSGGGGSWISAEFALDIVMTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWK

SEQ ID NO: 38 - protein, 271 aa, synthetic construct
  REGION 1..271: note = Synthetic Construct
MSVPTQVLGLLLLWLTDARCVKLQESGTELVKPGASVKLSCKASGYTFTSYWMHWVKQRPGQGLEWIGNINPSNGGTNYNEKFKSKATLTVDKSSSTAYMQLSSLTSEDSAVYYCARRGIRLRHFDYWGQGTTVTVSSGGGGSGGGGSGGGGSDIQMTQSPKFMSTSVGDRVSITCKASQDVGTAVAWYQQKPGQSPKLLIYWASTRHTGVPDRFTGSGSGTDFTLTISNVQSEDLADYFCQQYSSYPLTFGAGTKLELKRIEGRHHHHHH

SEQ ID NO: 39 - DNA, 324 bp, synthetic construct
  misc_feature 1..324: note = Synthetic Construct
  source 1..324: mol_type = other DNA
gacattgtgatgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaa

SEQ ID NO: 40 - DNA, 322 bp, synthetic construct
  misc_feature 1..322: note = Synthetic Construct
  source 1..322: mol_type = other DNA
gacatccagatgacccagtctcccaaattcatgtccacatcagtaggagacagggtcagcatcacctgcaaggccagtcaggatgtgggtactgctgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatcctctcacgttcggtgctgggaccaagctggagctgaaac

SEQ ID NO: 41 - DNA, 351 bp, synthetic construct
  misc_feature 1..351: note = Synthetic Construct
  source 1..351: mol_type = other DNA
gtgaaactgcaggagtctgggactgaactggtgaagcctggggcttcagtgaagctgtcctgcaaggcttctggctacaccttcaccagctactggatgcactgggtgaagcagaggcctggacaaggccttgagtggattggaaatattaatcctagcaatggtggtactaactacaatgagaagttcaagagcaaggccacactgactgtagacaaatcctccagcacagcctacatgcagctcagcagcctgacatctgaggactctgcggtctattattgtgcaagaagaggcatacggttacgacactttgactactggggccaagggaccacggtcaccgtctcc

SEQ ID NO: 42 - DNA, 828 bp, synthetic construct
  misc_feature 1..828: note = Synthetic Construct
  source 1..828: mol_type = other DNA
gccgccaccatgagtgtgcccactcaggtcctggggttgctgctgctgtggcttacagatgccagatgtgtgaaactgcaggagtctgggactgaactggtgaagcctggggcttcagtgaagctgtcctgcaaggcttctggctacaccttcaccagctactggatgcactgggtgaagcagaggcctggacaaggccttgagtggattggaaatattaatcctagcaatggtggtactaactacaatgagaagttcaagagcaaggccacactgactgtagacaaatcctccagcacagcctacatgcagctcagcagcctgacatctgaggactctgcggtctattattgtgcaagaagaggcatacggttacgacactttgactactggggccaagggaccacggtcaccgtctcctctggcggaggtgggtcgggtggcggcggatctggcggaggtgggtcggacatccagatgacccagtctcccaaattcatgtccacatcagtaggagacagggtcagcatcacctgcaaggccagtcaggatgtgggtactgctgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatcctctcacgttcggtgctgggaccaagctggagctgaaacggatcgaaggccggcatcaccatcatcaccactgatag

SEQ ID NO: 43 - DNA, 819 bp, synthetic construct
  misc_feature 1..819: note = Synthetic Construct
  source 1..819: mol_type = other DNA
atgagcgtgcctacacaggtgctcggcctgctgctcctctggctgacagacgcccggtgtgtgaagctgcaggagtccggaaccgagctggtgaaacctggcgccagcgtgaaactgagctgcaaagccagcggatacaccttcacctcctactggatgcactgggtgaaacagaggcctggccagggcctggaatggattggcaacatcaaccccagcaacggcggcaccaactacaatgagaagttcaagagcaaggccaccctgaccgtggataagtcctcctccaccgcctacatgcagctgtcctccctcacctccgaggacagcgccgtctattactgtgccaggcggggcatcaggctgaggcacttcgactactggggccaaggcacaaccgtcaccgtgagctccggaggaggaggcagcggaggcggaggctccggcggaggcggaagcgacattcagatgacccagagccccaagttcatgtccacctccgtcggcgacagggtgagcatcacctgtaaggccagccaggatgtcggcacagctgtggcctggtaccagcagaagcccggccagtcccccaagctgctgatctactgggcttccacaaggcataccggcgtccccgataggttcacaggctccggctccggcaccgacttcacactcaccatcagcaacgtccagtccgaggacctggccgactacttctgccagcagtactccagctaccccctcaccttcggcgctggcaccaagctggaactcaagcggatcgagggcaggcatcaccaccatcaccactgatag

SEQ ID NO: 44 - protein, 380 aa, synthetic construct
  REGION 1..380: note = Synthetic Construct
MRWLLLYYALCFSLSKASAHTVELNNMFGQIQSPGYPDSYPSDSEVTWNITVPDGFRIKLYFMHFNLESSYLCEYDYVKVETEDQVLATFCGRETTDTEQTPGQEVVLSPGSFMSITFRSDFSNEERFTGFDAHYMAVDVDECKEREDEELSCDHYCHNYIGGYYCSCRFGYILHTDNRTCRVECSDNLFTQRTGVITSPDFPNPYPKSSECLYTIELEEGFMVNLQFEDIFDIEDHPEVPCPYDYIKIKVGPKVLGPFCGEKAPEPISTQSHSVLILFHSDNSGENRGWRLSYRAAGNECPELQPPVHGKIEPSQAKYFFKDQVLVSCDTGYKVLKDNVEMDTFQIECLKDGTWSNKIPTCKKNEIDLESELKSEQVTE

SEQ ID NO: 45 - DNA, 1143 bp, synthetic construct
  misc_feature 1..1143: note = Synthetic Construct
  source 1..1143: mol_type = other DNA
atgaggtggctgcttctctattatgctctgtgcttctccctgtcaaaggcttcagcccacaccgtggagctaaacaatatgtttggccagatccagtcgcctggttatccagactcctatcccagtgattcagaggtgacttggaatatcactgtcccagatgggtttcggatcaagctttacttcatgcacttcaacttggaatcctcctacctttgtgaatatgactatgtgaaggtagaaactgaggaccaggtgctggcaaccttctgtggcagggagaccacagacacagagcagactcccggccaggaggtggtcctctcccctggctccttcatgtccatcactttccggtcagatttctccaatgaggagcgtttcacaggctttgatgcccactacatggctgtggatgtggacgagtgcaaggagagggaggacgaggagctgtcctgtgaccactactgccacaactacattggcggctactactgctcctgccgcttcggctacatcctccacacagacaacaggacctgccgagtggagtgcagtgacaacctcttcactcaaaggactggggtgatcaccagccctgacttcccaaacccttaccccaagagctctgaatgcctgtataccatcgagctggaggagggtttcatggtcaacctgcagtttgaggacatatttgacattgaggaccatcctgaggtgccctgcccctatgactacatcaagatcaaagttggtccaaaagttttggggcctttctgtggagagaaagccccagaacccatcagcacccagagccacagtgtcctgatcctgttccatagtgacaactcgggagagaaccggggctggaggctctcatacagggctgcaggaaatgagtgcccagagctacagcctcctgtccatgggaaaatcgagccctcccaagccaagtatttcttcaaagaccaagtgctcgtcagctgtgacacaggctacaaagtgctgaaggataatgtggagatggacacattccagattgagtgtctgaaggatgggacgtggagtaacaagattcccacctgtaaaaaaaatgaaatcgatctggagagcgaactcaagtcagagcaagtgacagagtga

SEQ ID NO: 46 - protein, 110 aa, synthetic construct
  REGION 1..110: note = Synthetic Construct
FGQIQSPGYPDSYPSDSEVTWNITVPDGFRIKLYFMHFNLESSYLCEYDYVKVETEDQVLATFCGRETTDTEQTPGQEVVLSPGSFMSITFRSDFSNEERFTGFDAHYMA

SEQ ID NO: 47 - DNA, 330 bp, synthetic construct
  misc_feature 1..330: note = Synthetic Construct
  source 1..330: mol_type = other DNA
tttggccagatccagtcgcctggttatccagactcctatcccagtgattcagaggtgacttggaatatcactgtcccagatgggtttcggatcaagctttacttcatgcacttcaacttggaatcctcctacctttgtgaatatgactatgtgaaggtagaaactgaggaccaggtgctggcaaccttctgtggcagggagaccacagacacagagcagactcccggccaggaggtggtcctctcccctggctccttcatgtccatcactttccggtcagatttctccaatgaggagcgtttcacaggctttgatgcccactacatggct

SEQ ID NO: 48 - protein, 38 aa, synthetic construct
  REGION 1..38: note = Synthetic Construct
DVDECKEREDEELSCDHYCHNYIGGYYCSCRFGYILHT

SEQ ID NO: 49 - DNA, 114 bp, synthetic construct
  misc_feature 1..114: note = Synthetic Construct
  source 1..114: mol_type = other DNA
gatgtggacgagtgcaaggagagggaggacgaggagctgtcctgtgaccactactgccacaactacattggcggctactactgctcctgccgcttcggctacatcctccacaca

SEQ ID NO: 50 - protein, 112 aa, synthetic construct
  REGION 1..112: note = Synthetic Construct
CSDNLFTQRTGVITSPDFPNPYPKSSECLYTIELEEGFMVNLQFEDIFDIEDHPEVPCPYDYIKIKVGPKVLGPFCGEKAPEPISTQSHSVLILFHSDNSGENRGWRLSYRA

SEQ ID NO: 51 - DNA, 336 bp, synthetic construct
  misc_feature 1..336: note = Synthetic Construct
  source 1..336: mol_type = other DNA
tgcagtgacaacctcttcactcaaaggactggggtgatcaccagccctgacttcccaaacccttaccccaagagctctgaatgcctgtataccatcgagctggaggagggtttcatggtcaacctgcagtttgaggacatatttgacattgaggaccatcctgaggtgccctgcccctatgactacatcaagatcaaagttggtccaaaagttttggggcctttctgtggagagaaagccccagaacccatcagcacccagagccacagtgtcctgatcctgttccatagtgacaactcgggagagaaccggggctggaggctctcatacagggct

SEQ ID NO: 52 - protein, 63 aa, synthetic construct
  REGION 1..63: note = Synthetic Construct
CPELQPPVHGKIEPSQAKYFFKDQVLVSCDTGYKVLKDNVEMDTFQIECLKDGTWSNKIPTCK

SEQ ID NO: 53 - DNA, 189 bp, synthetic construct
  misc_feature 1..189: note = Synthetic Construct
  source 1..189: mol_type = other DNA
tgcccagagctacagcctcctgtccatgggaaaatcgagccctcccaagccaagtatttcttcaaagaccaagtgctcgtcagctgtgacacaggctacaaagtgctgaaggataatgtggagatggacacattccagattgagtgtctgaaggatgggacgtggagtaacaagattcccacctgtaaa

SEQ ID NO: 54 - protein, 21 aa, Homo sapiens
MPMGSLQPLATLYLLGMLVAS

SEQ ID NO: 55 - protein, 17 aa, Homo sapiens
MGAAGLLGVFLALVAPG

SEQ ID NO: 56 - protein, 20 aa, Homo sapiens
MGAAGLLGVFLALVAPGVLG

SEQ ID NO: 57 - DNA, 927 bp, synthetic construct
  misc_feature 1..927: note = Synthetic Construct
  source 1..927: mol_type = other DNA
gacacgtgatcagccgccaccatgcccatggggtctctgcaaccgctggccaccttgtacctgctggggatgctggtcgcttccgtgctagcgcatcatcatcatcatcatgtgaaactgcaggagtcaggggctgagcttgtgaagcctggggcttcagtgaagctgtcctgcaaggcttctggctacaccttcaccagctactggatgcactgggtgaagcagaggcctggacgaggccttgagtggattggaaggattggtcctaatagtggtggtactaagtacaatgagaagttcaagagcaaggccacactgactgtagacaaaccctccagcacagcctacatgcagctcagcagcctgacatctgaggactctgcggtctattattgtgcaagaagaatggtaaaggggtgctatggactactggggccaagggaccacggtcaccgtctcctcaaagggcgaattccagcacactggcggccgttactagtggatccgagctcggtaccaagcttggcgtcaggaggcggtggcggctcgggtggcggcggctcttggatatctgcagaattcgcccttgacattgagctcacccagtctccaaccaccatggctgcatctcccggggagaagatcactatcacctgcagtgccagctcaagtataagttccaattacttgcattggtatcagcagaagccaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtagtagtataccacgtacacgttcggagggggcaccaagctggaaataatagactagtcgtgcg

SEQ ID NO: 58 - DNA, 954 bp, synthetic construct
  misc_feature 1..954: note = Synthetic Construct
  source 1..954: mol_type = other DNA
gacacgaagcttgccgccaccatgcccatggggtctctgcaaccgctggccaccttgtacctgctggggatgctggtcgcttccgtgctagcgcatcatcatcatcatcatgtcaagctgcaggagtctgggactgaactggtgaagcctggggcttcagtgaagctgtcctgcaaggcttctggctacaccttcaccagctactggatgcactgggtgaagcagaggcctggacaaggccttgagtggattggaaatattaatcctagcaatggtggtactaactacaatgagaagttcaagagcaaggccacactgactgtagacaaatcctccagcacagcctacatgcagctcagcagcctgacatctgaggactctgcggtctattattgtgcaagaagaggcatacggttacgacactttgactactggggccaagggaccacggtcaccgtctcctcaagggcgaattctgcagatatccatcacactggcggccgctcgagcatgcatctagagggcccaattcgccctatagtgagtcgtatatcaggaggcggtggcggctcgggtggcggcggctcttggatatctgcagaattcgcccttgacattgtgatgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaataatagcccgggcgtgcg

SEQ ID NO: 59 - protein, 9 aa, synthetic construct
  REGION 1..9: note = Synthetic Construct
YPYDVPDYA

(19) United States
(12) Patent Application Publication
(10) Pub. No.: US 2025/0000955 A1
(43) Pub. Date: Jan. 2, 2025

(54) MAP44 POLYPEPTIDES AND CONSTRUCTS BASED ON NATURAL ANTIBODIES AND USES THEREOF

(71) Applicant: The Regents of the University of Colorado, A Body Corporate, Denver, CO (US)

(72) Inventors: V. Michael Holers, Denver, CO (US); Nirmal Banda, Aurora, CO (US); Liudmila Kulik, Denver, CO (US)

(21) Appl. No.: 18/614,952

(22) Filed: Mar. 25, 2024

Related U.S. Application Data
(63) Continuation of application No. 18/485,947, filed on Oct. 12, 2023, now abandoned.
Application No. 18/485,947 is a continuation of application No. 17/113,612, filed on Dec. 7, 2020, now abandoned.
Application No. 17/113,612 is a continuation of application No. 15/316,113, filed on Dec. 2, 2016, now abandoned.
(60) Provisional application No. 62/008,470, filed on Jun. 5, 2014.

(51) Int. Cl. A61K 38/48; A01K 67/027; A61K 38/00; A61K 48/00; C07K 16/18; C07K 16/44; C12N 9/64

(52) U.S. Cl. CPC A61K 38/482; A01K 67/027; C07K 16/18; C07K 16/44; C12N 9/6424; C12Y 304/21; A01K 2207/10; A01K 2227/105; A61K 38/00; A61K 48/00; C07K 2317/622; C07K 2319/00; C12N 2710/10041

(57) ABSTRACT

The present invention provides delivery methods and constructs for treating inflammatory diseases in an individual. The targeted delivery approach utilizes an antibody that recognizes an epitope found to be present at sites of inflammation. The antibody is used to deliver a MAp44 polypeptide or fragment thereof to sites of inflammation, where it inhibits the lectin pathway of complement activation.

This application is a continuation application of U.S. patent application Ser. No. 18/485,947, filed Oct. 12, 2023, which is a continuation application of U.S. patent application Ser. No. 17/113,612, filed Dec. 7, 2020 and published as US 2021-0213110 A1 on Jul. 15, 2021, which is a continuation of U.S. patent application Ser. No. 15/316,113, filed on Dec. 2, 2016 and published as US 2017-0209549 A1 on Jul. 27, 2017, which is a U.S. National Stage Application of International Patent Application No. PCT/US2015/34270, filed on Jun. 4, 2015 and published as WO 2015/187992 on Dec. 10, 2015, which claims the benefit of the filing date under 35 U.S.C. § 119(e) to U.S. Provisional Patent Application No. 62/008,470, filed on Jun. 5, 2014; the disclosure of each of the above-referenced applications, including any drawings, sequence listings, and biological material deposit statements, are incorporated herein by reference in their entirety.

### STATEMENT REGARDING FEDERALLY SPONSORED RESEARCH OR DEVELOPMENT

This invention was made with government support under grant number AR051749 awarded by The National Institutes of Health. The government has certain rights in the invention.

### TECHNICAL FIELD

This application pertains to MAp44 polypeptides or fragments thereof optionally linked to a targeting moiety and methods for their use.

### SEQUENCE LISTING

The instant application contains a Sequence Listing XML file which has been submitted electronically in XML format and is hereby incorporated by reference in its entirety. Said XML copy, created Mar. 25, 2024, is named 132301-00304.xml and is 81,203 bytes in size.

### BACKGROUND OF THE INVENTION

The complement system is a central part of the innate immune system. Activation of the complement system is thought to contribute to inflammation and tissue damage in human rheumatoid arthritis (RA), especially in very early disease (Okroj et al., 2007, Ann. Med. 39: 517-530; Sturfelt and Truedsson, 2012, Nat. Rev. Rheumatol. 8: 458-468; Zvaifler, 1974, Arthritis Rheum. 17: 297-305). RA is a complex autoimmune disease with genetic and environmental components, affecting approximately 1% of the population worldwide (Helmick et al., 2008, Arthritis and rheumatism 58: 15-25). Autoantibodies, especially as constituents of immune complexes (ICs), play a central role in triggering inflammation in this disease (Arend and Firestein, 2012, Nat. Rev. Rheumatol. 8: 573-586; Klareskog et al., 2008, Annual review of immunology 26: 651-675). The complement system has been found to play a major role in the development of inflammation and tissue damage in collagen antibody-induced arthritis (CAIA) and other animal models of inflammatory arthritis (Banda et al., 2006, J. Immunol. 177: 1904-1912; Hietala et al., 2002, J. Immunol. 169: 454-459; Ji et al., 2002, Immunity 16: 157-168; Wang et al., 2000, J. Immunol. 164: 4340-4347). ICs containing Abs of the IgG isotype are found in the cartilage and synovium of the joints of patients with RA and have been implicated in induction of local tissue damage through activation of the complement system (Cooke et al., 1975, Arthritis Rheum. 18: 541-551; Ghose et al., 1975, J. Clin. Pathol. 28: 109-117; Ohno and Cooke, 1978, Arthritis Rheum. 21: 516-527).

The complement system can be activated by three pathways: the classical pathway (CP), the lectin pathway (LP), and the alternative pathway (AP). IgG Abs in arthritis-related IC in human RA have previously been shown to activate both the CP and AP of the complement system (Banda et al., 2008, Arthritis Rheum. 58: 3081-3089; Ratnoff et al., 1983, Springer Semin. Immunopathol. 6: 361-371; Wouters et al., 2006, Arthritis Rheum. 54: 1143-1150). In CAIA, using pathway-specific functional deficiencies of the complement system developed through gene targeting and inactivation of specific pathway components, it was previously concluded that the AP alone is both necessary and sufficient for the development of CAIA through its role in both the initiation process and amplification loop (Banda et al., 2006, J. Immunol. 177: 1904-1912). The lack of a role for the LP was inferred using MBL-A/C and C4 deficient mice (Banda et al., 2006, J. Immunol. 177: 1904-1912; Ji et al., 2002, Immunity 16: 157-168; Banda et al., 2007, J. Immunol. 179: 4101-4109), and for the CP using C4 and C1q deficient mice (Banda et al., 2006, J. Immunol. 177: 1904-1912; Banda et al., 2007, J. Immunol. 179: 4101-4109), where disease was largely unchanged.

The CP is initiated by C1q binding to Ab in an IC, leading to activation of C1r, C1s and to the subsequent formation of the CP C3 convertase, C4b2b. The LP is initiated when members of a family of pattern recognition molecules designated the collectins, whose members are mannose-binding lectin (MBL), ficolins (the three in humans are designated H, L, and M), and Collectin-11 (also termed CL-K1) bind along, with MBL-associated serine proteases (MASP-1, MASP-2, and MASP-3), to arrays of specific monosaccharides or modified carbohydrates present on the surface of microorganisms and other target surfaces and molecules. Notably, in humans one MBL is found whereas in mice two, MBL-A and MBL-C, are present; in addition, two ficolins (Ficolin-A and Ficolin-B) are found in mice along with Collectin-11 (Hansen et al., 2000, J. Immunol. 164: 2610-2618; Kawai et al., 2002, Bioscience, biotechnology, and biochemistry 66: 2134-2145; Ohashi and Erickson, 1998, Archives of biochemistry and biophysics 360: 223-232; Ohtani et al., 1999, J. Biol. Chem. 274: 13681-13689). This process leads to the formation of the shared CP/LP C3 convertase, C4b2b, through the activities of MASP-2 (Thiel et al., 1997, Nature 386: 506-510) in a manner that requires initial engagement of MASP-1 (Moller-Kristensen et al., 2007, Int. Immunol. 19: 141-149; Heja et al., 2012, Proc. Natl. Acad. Sci. USA 109: 10498-10503). The AP is initiated by spontaneous turnover of C3 with transient formation of hydrolyzed C3 (H₂O), followed by binding of factor B (FB) with cleavage by factor D (FD) and generation of the AP initiation C3 convertase C3(H₂O)Bb (Pangburn et al., 1983, J. Immunol. 131: 1930-1935). Cleavage of C3 also exposes a short-lived thioester in C3b that covalently attaches to amine and carboxyl groups on target surfaces (Pangburn et al., 1983, J. Immunol. 131: 1930-1935). Following formation of C3b through any of the three pathways, the amplification loop is initiated through the binding of FB and cleavage by FD to form the C3bBb C3 convertase (Rosen et al., 1989, Science 244: 1483-1487).

The AP may also be initiated by properdin bound to target-containing molecular patterns (Kemper et al., 2010, Annu. Rev. Immunol. 28: 131-155) or by adherent IgG or IgA (Wouters et al., 2006, Arthritis Rheum. 54: 1143-1150; Hiemstra et al., 1988, Mol. Immunol. 25: 527-533). In addition, it was reported that the AP in mice is dependent on MASP-1/3 cleavage of pro-FD to form mature FD in the circulation (Takahashi et al., 2010, J. Exp. Med. 207: 29-37) and that mice lacking MASP-1 and MASP-3 have a defective AP and LP (Takahashi et al., 2008, J. Immunol. 180: 6132-6138). Recently, it has been shown that MASP-1/3−/−/fH−/− mice have pro-DF in their circulation and the AP was present but still somewhat defective (Ruseva et al., 2013, Clin. Exp. Immunol.). However, as opposed to mice, a functional AP was present in the serum of a patient reported to lack MASP-1 and MASP-3 (Degn et al., 2012, J. Immunol. 189: 3957-3969).

MBL, ficolins and Collectin-11 circulate in complex with MASP-1, -2 and -3 and two additional proteins (MAp19 and MAp44, also known as sMAP and MAP1, respectively) (Degn et al., 2012, J. Immunol. 189: 3957-3969; Degn et al., 2010, J. Immunol. Methods 361: 37-50). MASPs are present as pro-enzymes and become activated once MBL, ficolins or Collectin-11 bind to ligands. Three proteins, MASP-1, MASP-3 and MAp44 are translated from mRNAs formed by alternative splicing of RNA encoded by the MASP-1 gene (Degn et al., 2012, J. Immunol. 189: 3957-3969). MASP-1 and MASP-3 are two proteases which share their first five domains (CUB1-EGF-CUB2-CCP1-CCP2) but have different serine protease domains encoded by distinct exons (Dahl et al., 2001, Immunity 15: 127-135). MAp44 shares the first four domains with MASP-1 and MASP-3, followed by 17 unique C-terminal amino acid residues encoded by a separate exon (Degn et al., 2010, J. Immunol. Methods 361: 37-50). Since the first three domains mediate binding to MBL, MAp44, MASP-1 and MASP-3 bind to the same site on MBL. MAp44, which lacks a serine protease domain, can thus compete with MASPs for binding to MBL and other collectins, and through this mechanism regulate activity of the LP (Degn et al., 2009, J. Immunol. 183: 7371-7378). MASP-2 activation strictly depends on an initiating activation of MASP-1 because inhibition of MASP-1 prevents autoactivation of MASP-2 (Heja et al., 2012, Proc. Natl. Acad. Sci. USA 109: 10498-10503), and no LP is present in mice lacking MASP-1 (Takahashi et al., 2008, J. Immunol. 180: 6132-6138). MAp44 may also displace MASP-1 and MASP-2 from MBL or ficolins, further inhibiting the activation of MASP-2 and the subsequent cleavage of C4 and C2 (Degn et al., 2009, J. Immunol. 183: 7371-7378). Through these activities, MAp44 is considered to be a natural endogenous inhibitor of the LP (Pavlov et al., 2012, Circulation 126: 2227-2235).

Previously, studies in mice deficient in different components of the complement system have shown that the AP is both necessary and sufficient to mediate CAIA as neither the LP nor the CP appear to be required (Banda et al., 2006, J. Immunol. 177: 1904-1912; Banda et al., 2007, J. Immunol. 179: 4101-4109). Additionally, mice lacking MASP-1, MASP-3 and MAp44 (MASP-1/3−/−) were shown to be resistant to CAIA (Banda et al., 2010, J. Immunol. 185: 5598-5606), likely because they lacked mature FD and a functional AP (Takahashi et al., 2008, J. Immunol. 180: 6132-6138). Although the AP may initiate and amplify CAIA, the LP (and CP) may also function to initiate the disease process. Since the LP has not previously been shown to play an essential role in CAIA, we hypothesized that ficolin-A or —B or Collectin-11 may mediate recognition of ligands independently of MBL-A/C. In addition, direct MASP-mediated cleavage of C3 (Matsushita and Fujita, 1995, Immunobiology 194: 443-448) could have allowed C3 activation and engagement of the amplification loop even in the absence of MBL-A/C or C4. The use of MAp44 as an endogenous inhibitor of all of the MASPs, due to its interference with the interactions between the recognition molecules and MASPs, should allow a more complete impairment of the LP.

The effectiveness of MAp44 as a therapeutic agent could be increased by its targeting to sites of complement activation related to tissue injury or disease. Natural antibodies exist in an immune competent individual and can be found in the serum or plasma of an individual not known to have been stimulated by a specific antigen to which the antibody binds. Previous studies by the present inventors and colleagues have shown that certain types of natural antibodies recognize epitopes on ischemic tissue and catalyze the initiation and subsequent development of ischemia-reperfusion injury (Fleming et al., 2002, J. Immunol. 169:2126-2133; Rehrig et al., 2001, J. Immunol. 167:5921-5927). Ischemia-reperfusion injury, as well as hypovolemic shock and subsequent tissue damage, is known to be caused by complement and Fc receptor activation and the recruitment and activation of neutrophils and other inflammatory cells (Rehrig et al., 2001, supra). It had also been shown that single monoclonal antibodies that react broadly with phospholipids and other extracellular or intracellular antigens such as DNA can cause ischemia-reperfusion injury in mice that lack other antibodies (i.e., B cell-deficient mice).

Ischemia-reperfusion (IR) injury refers to damage to a tissue caused when the blood supply returns to the tissue after a period of ischemia (restriction in blood supply). The absence of oxygen and nutrients from the blood creates a condition in which the restoration of circulation results in inflammation and oxidative damage, rather than restoration of normal function. Ischemia-reperfusion injury can be associated with traumatic injury, including hemorrhagic shock, as well as many other medical conditions such as stroke or large vessel occlusion, and is a major medical problem. More particularly, ischemia-reperfusion injury is important in heart attacks, stroke, kidney failure following vascular surgery, post-transplantation injury and chronic rejection, as well as in various types of traumatic injury, where hemorrhage will lead to organ hypoperfusion, and then subsequent reperfusion injury during fluid resuscitation. Ischemia-reperfusion injury, or an injury due to reperfusion and ischemic events, is also observed in a variety of autoimmune and inflammatory diseases. Independently of other factors, ischemia-reperfusion injury leads to increased mortality.

There is also increasing evidence of reperfusion injury that can be found in autoimmune and inflammatory diseases that are not traditionally thought of as reperfusion injury-related. For example, the synovium in rheumatoid arthritis patients is a site that is subjected to constant reperfusion stress (e.g., low pH, lots of tissue pressure and poor perfusion). The higher quantity of synovial fluid found in hypermobile patients having this disease causes an increase in the intra-articular pressure, which is then exacerbated by joint motion. This may aggravate local inflammation through a hypoxic/reperfusion mechanism, which in turn causes oxidative injury due to intermittent ischemia (e.g., see Punzi et al., Rheumatology 2001; 40: 202-204; Pianon et al., Reumatismo 1996; 48(Suppl. 1):93; and Jawed et al., Ann Rheum Dis 1997; 56:686-9). A variety of inflammatory and autoimmune diseases can also be associated with similar changes in cell stress responses of local cells that are similar to, or mimic, some changes in reperfusion injury.

Kulik et al. showed that pathogenic natural antibodies recognizing Annexin IV are required to develop intestinal ischemia-reperfusion injury. J. Immunol. 2009; 182:5363-5373. U.S. Patent Application Publication No. 2011/0014270 discloses lipids, annexins, and lipid-annexin complexes for use in the prevention and/or treatment of ischemia-reperfusion injury and reperfusion injury associated with a variety of diseases and conditions.

The disclosures of all publications, patents, patent applications and published patent applications referred to herein are hereby incorporated herein by reference in their entirety.

### BRIEF SUMMARY OF THE INVENTION

In one aspect, the present disclosure provides a method of treating a complement-mediated disease in an individual, comprising administering to the individual an effective amount of a composition comprising a construct, wherein the construct comprises a MAp44 polypeptide or fragment thereof.

In some embodiments, the complement-mediated disease is arthritis.

In some embodiments, the MAp44 polypeptide or fragment thereof comprises the sequence of SEQ ID NO: 44. In some embodiments, the MAp44 polypeptide or fragment thereof is between about 50 and about 380 amino acids in length, and comprises a continuous sequence in SEQ ID NO: 44. In some embodiments, the MAp44 polypeptide or fragment thereof comprises amino acids 1-137, amino acids 1-176, amino acids 1-296, or amino acids 1-363 of SEQ ID NO: 44. In some embodiments, the MAp44 polypeptide or fragment thereof comprises one or more sequences selected from the group consisting of SEQ ID NOs: 46, 48, 50 and 52.

In some embodiments, the construct further comprises a targeting moiety, such as an antibody or fragment (e.g., an antigen-binding fragment) thereof, for example a naturally occurring antibody or fragment thereof. In some embodiments, the naturally occurring antibody or fragment thereof recognizes Annexin IV or a phospholipid, such as naturally occurring antibodies B4 or C2.

In some embodiments, the antibody or fragment thereof specifically binds to Annexin IV. In some embodiments, the antibody or fragment thereof competitively inhibits the binding of monoclonal antibody B4 to Annexin IV. In some embodiments, the antibody or fragment thereof binds to the same epitope as monoclonal antibody B4. In some embodiments, the Annexin IV is present on the surface of a cell in an individual that is in or adjacent to a tissue undergoing injury.

In some embodiments, the antibody or fragment thereof specifically binds to a phospholipid. In some embodiments, the antibody or fragment thereof competitively inhibits the binding of monoclonal antibody C2 to the phospholipid. In some embodiments, the antibody or fragment thereof binds to the same epitope as monoclonal antibody C2. In some embodiments, the phospholipid is present on the surface of a cell in an individual that is in or adjacent to a tissue undergoing tissue injury and/or oxidative damage. In some embodiments, the phospholipid is selected from the group consisting of phosphatidylethanolamine (PE), cardiolipin (CL), and phosphatidylcholine (PC). In some embodiments, the antibody or fragment thereof binds to malondialdehyde (MDA).

In some embodiments of any one of the methods described above, the construct is a fusion protein. In some embodiments, the antibody or fragment thereof and the MAp44 polypeptide or fragment thereof are linked via a peptide linker. In some embodiments, the antibody or fragment thereof and the MAp44 polypeptide or fragment thereof are directly linked.

In some embodiments of the method, the antibody or fragment thereof is an scFv. In some embodiments, the antibody or fragment thereof is a Fab, Fab′, or F(ab′)2.

In another aspect, the present disclosure provides a construct comprising a non-naturally occurring MAp44 fragment, wherein the MAp44 fragment comprises at least about 50 continuous amino acids of the sequence of SEQ ID NO: 44. In some embodiments, the MAp44 fragment is between about 50 and about 350 amino acids in length. In some embodiments, the MAp44 fragment comprises amino acids 1-137, amino acids 1-176, amino acids 1-296, or amino acids 1-363 of SEQ ID NO: 44. In some embodiments, the MAp44 polypeptide or fragment thereof comprises one or more sequences selected from the group consisting of SEQ ID NOs: 46, 48, 50 and 52.

In some embodiments of the construct, the construct further comprises an antibody or fragment thereof, wherein the antibody or fragment thereof specifically binds to Annexin IV and comprises: (i) a light chain variable domain comprising a sequence (e.g., a light chain CDR1 sequence) of SEQ ID NO: 1 or 7, a sequence (e.g., a light chain CDR2 sequence) of SEQ ID NO: 2 or 8, or a sequence (e.g., a light chain CDR3 sequence) of SEQ ID NO: 3 or 9; and/or (ii) a heavy chain variable domain comprising a sequence (e.g., a heavy chain CDR1 sequence) of SEQ ID NO: 4 or 10, a sequence (e.g., a heavy chain CDR2 sequence) of SEQ ID NO: 5 or 11, or a sequence (e.g., a heavy chain CDR3 sequence) of SEQ ID NO: 6 or 12. In some embodiments, the antibody or fragment thereof comprises a light chain variable domain comprising a sequence of SEQ ID NO: 1 or 7, a sequence of SEQ ID NO: 2 or 8, and a sequence of SEQ ID NO: 3 or 9. In some embodiments, the antibody or fragment thereof comprises a heavy chain variable domain comprising a sequence of SEQ ID NO: 4 or 10, a sequence of SEQ ID NO: 5 or 11, and a sequence of SEQ ID NO: 6 or 12.

In some embodiments of the construct, the antibody or fragment thereof comprises a light chain variable domain of SEQ ID NO: 13 or 14. In some embodiments, the antibody or fragment thereof comprises a heavy chain variable domain of SEQ ID NO: 15 or 16. In some embodiments, the antibody or fragment is an scFv having the sequence of SEQ ID NO: 17 or 18.

In some embodiments of the construct, the antibody or fragment thereof competitively inhibits the binding of monoclonal antibody B4 to Annexin IV. In some embodiments, the antibody or fragment thereof binds to the same epitope as monoclonal antibody B4. In some embodiments, the Annexin IV is present on the surface of a cell in an individual that is in or adjacent to a tissue undergoing or at risk of undergoing tissue injury.

In some embodiments of the construct, the construct comprises an antibody or fragment thereof, wherein the antibody or fragment thereof specifically binds to a phospholipid and comprises: (i) a light chain variable domain comprising a sequence (e.g., a light chain CDR1 sequence) of SEQ ID NO: 25 or 31, a sequence (e.g., a light chain CDR2 sequence) of SEQ ID NO: 26 or 32, or a sequence (e.g., a light chain CDR3 sequence) of SEQ ID NO: 27 or 33; and/or (ii) a heavy chain variable domain comprising a sequence (e.g., a heavy chain CDR1 sequence) of SEQ ID NO: 28, a sequence (e.g., a heavy chain CDR2 sequence) of SEQ ID NO: 29, or a sequence (e.g., a heavy chain CDR3 sequence) of SEQ ID NO: 30. In some embodiments, the antibody or fragment thereof comprises a light chain variable domain comprising a sequence of SEQ ID NO: 25 or 31, a sequence of SEQ ID NO: 26 or 32, and a sequence of SEQ ID NO: 27 or 33. In some embodiments, the antibody or fragment thereof comprises a heavy chain variable domain comprising a sequence of SEQ ID NO: 28, a sequence of SEQ ID NO: 29, and a sequence of SEQ ID NO: 30.

In some embodiments of the construct, the antibody or fragment thereof comprises a light chain variable domain of SEQ ID NO: 34 or 35. In some embodiments, the antibody or fragment thereof comprises a heavy chain variable domain of SEQ ID NO: 36. In some embodiments, the antibody or fragment is an scFv having the sequence of SEQ ID NO: 37 or 38.

In some embodiments of the construct, the antibody or fragment thereof competitively inhibits the binding of monoclonal antibody C2 to the phospholipid. In some embodiments, the antibody or fragment thereof binds to the same epitope as monoclonal antibody C2. In some embodiments, the phospholipid is present on the surface of a cell in an individual that is in or adjacent to a tissue undergoing or at risk of undergoing tissue injury. In some embodiments, the phospholipid is selected from the group consisting of phosphatidylethanolamine (PE), cardiolipin (CL), and phosphatidylcholine (PC). In some embodiments, the antibody or fragment thereof binds to MDA.

In some embodiments of the construct, the construct is a fusion protein. In some embodiments, the antibody or fragment thereof and the MAp44 fragment are linked by a peptide linker. In some embodiments, the antibody or fragment thereof and the MAp44 fragment are directly linked.

In another aspect, the present disclosure provides a pharmaceutical composition comprising any one of the constructs described above.

In another aspect, the present disclosure provides a method of treating a complement-mediated disease in an individual, comprising administering to the individual an effective amount of a pharmaceutical composition comprising any one of the constructs described above. In some embodiments, the method of treating a complement-mediated disease in an individual comprises administering to the individual a vector comprising an exogenous nucleic acid comprising a sequence for expression of a construct described above. In some embodiments, the vector is chosen from the group consisting of an adenovirus, a retrovirus, an adeno-associated virus, and a plasmid. In some embodiments, the vector is an adenovirus.

Also provided are unit dosage forms, kits, and articles of manufacture that are useful for the methods described herein.

It is to be understood that one, some, or all of the properties of the various embodiments described herein may be combined to form other embodiments of the present invention.

### BRIEF DESCRIPTION OF THE FIGURES

FIGS. 1A-ID. Substantial decrease in the CDA of anti-CII mAb-induced arthritis by pretreatment with AdhMAp44. Higher (HD) and lower (LD) doses of AdhMAp44 particles were used. FIG. 1A. Prevalence of arthritis (%) over the duration of the experiment. FIG. 1B. CDA over the duration of the experiment. Black arrows show the injection time of AdhMAp44 or AdGFP at days −5, 0 and 3. *p<0.05 in comparison with AdGFP treatment for data in B. FIG. 1C. All joint mean (AJM) histopathologic scores for inflammation (black solid bar), pannus formation (white hatched bar), cartilage damage (white empty bar) and bone damage (white line bar) from the five joints (2 forepaws, right hind knee, right hind ankle and right hind paw) were performed following tissue processing and toluidine-blue staining of sections. FIG. 1D. AJM for C3 deposition in the five joints examined within the synovium (black solid bar), on the surface of cartilage (white empty bar), and total score (white line bar) is presented. The data are expressed as mean of disease±SEM (n=5 each time point).

FIGS. 2A-2H. Representative histopathology and C3 deposition images from the knee joints of WT mice treated with AdGFP or AdhMAp44 (HD). The top two panels from left to right (FIGS. 2A & 2C) show staining with toluidine-blue from the knee joints of WT mice treated with AdGFP (left panel) and AdhMAp44 (right panel). The second two panels from left to right (FIGS. 2B & 2D) show staining with Toluidine-blue from the ankle joints of WT mice treated with AdGFP (left panel) and AdhMAp44 (right panel). The third set of two panels from left to right (FIGS. 2E & 2G) show staining with anti-C3 Ab from the knee joints of WT mice treated with AdGFP (left panel) and AdhMAp44 (right panel). The fourth set of two panels from left to right (FIGS. 2F & 2H) show staining with anti-C3 Ab from the ankle joints of WT mice treated with AdGFP (left panel) and AdhMAp44 (right panel). Areas of synovium (S-black arrow), cartilage (C-black arrow), bone (B) and meniscus (M) are identified. Data from using a LD or a HD of AdhMAp44 in the study were indistinguishable; therefore, we show representative pictures from only the HD of AdhMAp44. Magnification for all knee joint images shown in FIG. 2 is 20×, and magnification for all ankle joints shown in FIG. 2 is 10×. Scale bar is 0.1 mm (100 μm).

FIGS. 3A-3D. Effect of AdhMAp44 on C5a levels and C3 activation. FIG. 3A. Decrease in the absolute levels of C5a in the circulation of AdhMAp44 treated mice. Mice were injected i.p. with PBS, AdGFP or AdhMAp44. The absolute levels of C5a were measured using sera from mice before (day −5) after the induction of CAIA (day 10). The data in A represent the mean±SEM based on n=5 for each group. LD=low dose AdhMAp44. HD=high dose AdhMAp44. *p<0.05 in comparison with AdGFP treatment. FIG. 3B. ELISA showing a decrease in mannan-induced (LP) C3 activation, in vitro, using sera from AdhMAp44 treated mice. Sera obtained from mice injected i.p. with PBS, AdGFP or AdhMAp44, at day 10, were analyzed for C3 activation in vitro. Sera from WT mice (No Tx), without CAIA, were used as a positive control. Sera from C3−/− and MBUDf−/− mice were used as negative controls. The data in FIG. 3B represent mean±SEM based on WT mice with no Tx and no CAIA (n=4), C3−/− (n=4) and MBUDf−/− (n=4). *p<0.05 in comparison with AdGFP treatment. FIG. 3C. ELISA showing an overall decrease in LPS-induced C3 activation in vitro in GVB buffer with Ca⁺⁺ (all complement pathways are active) by recombinant human MAp44 in vitro. WT sera from mice without any CAIA were pre-treated with rhMAp44 or anti-fB inhibitory antibody. FIG. 3D. ELISA showing a decrease in LPS-induced C3 activation in Ca-deficient buffer with Mg⁺⁺ EGTA (AP only is active) by recombinant human MAp44 in vitro. WT sera from mice without any CAIA were pre-treated with rhMAp44 or anti-fB inhibitory antibody. The data in FIGS. 3C & 3D represent the mean±SEM based on n=4 for each treatment group. FIG. 3C GVB+ buffer; and FIG. 3D Mg2+ EGTA buffer. p<0.05 in comparison with no Tx sera from WT mice.

FIGS. 4A-4D. Levels of human MAp44 in the circulation of CAIA mice treated with or without AdMAp44. FIG. 4A. Levels human MAp44 in the circulation of mice at day −5 prior to injecting i.p. with PBS or AdGFP or LD and HD of AdhMAp44. FIG. 4B. Levels of human MAp44, at day 0, in the circulation of mice injected with PBS or AdGFP or AdhMAp44. FIG. 4C. Levels human MAp44 at day 3 injected with PBS or AdGFP or AdhMAp44. FIG. 4D. Levels at day 10 of human MAp44 in the circulation of mice with CAIA injected with PBS or AdGFP or AdhMAp44. ELISAs were performed for human MAp44. Sera from mice with CAIA injected with LD or HD dose of AdhMAp44 exhibit somewhat similar levels of human MAp44. In contrast, mice injected with PBS or AdGFP have no detectable levels of human MAp44. The data represent the mean±SEM (ng/ml) based on n=5 for each group, except for mice injected with a HD of AdhMAp44 (n=4). *p<0.01 in comparison with PBS or AdGFP treatment.

FIGS. 5A-5B. Decrease in the CDA resulting from pretreatment with AdmMAp44 in CAIA. The data shown are derived from the indicated days after anti-CII mAb and LPS injections. The arrows in panels A and B indicate the days of injection of AdGFP or AdmMAp44. FIG. 5A. Prevalence of arthritis (%) over the duration of the experiment. FIG. 5B. CDA over the duration of the experiment. The data represent the mean±SEM for each group (n=5). Arrows indicate the injection days of AdmMAp44 and AdGFP. *p<0.05 in comparison to AdGFP treatment.

FIGS. 6A-6D. Decrease in scoring for inflammation, pannus, cartilage damage, and bone damage as well as staining for C3 deposition in knee joints of mice with CAIA treated with AdmMAp44 compared to AdGFP. FIG. 6A. AJM of histopathologic score for inflammation (black solid bar), pannus formation (white hatched bar), cartilage damage (white empty bar) and bone damage (white line bar) from the five joints (2 forepaws, right hind knee, right hind ankle and right hind paw) was performed following tissue processing and toluidine-blue staining of sections. FIG. 6B. Pearson correlation (r) between histology scores (total scores) and CDA. FIG. 6C. AJM for C3 deposition score from the five joints in the synovium (black solid bar), on the surface of cartilage (white empty bar) and total score (white line bar) is presented. FIG. 6D. Pearson correlation (r) between C3 deposition total scores (synovium and cartilage) and CDA. The data are expressed as mean of disease±SEM (n=5).

FIGS. 7A-7F Decrease in the overall CDA by local right knee joint injection of AdmMAp44 or AdGFP on arthritis induced by anti-CII mAb. WT mice were injected three times locally in the right knee joint at days −5, 0, and 3. The effects were examined on both forepaws and the left hind limb. The data shown are derived from the indicated days after the mAb and LPS injection. FIG. 7A. CDA in all joints over the duration of the experiment. FIG. 7B. Prevalence of arthritis (%) in all joints over the duration of the experiment. FIG. 7C. CDA in the right knee joint over the duration of the experiment. FIG. 7D. CDA in the left hind limb over the duration of the experiment. FIG. 7E. CDA in the right fore paw over the duration of the experiment. FIG. 7F. CDA in the left forepaw over the duration of the experiment. The data represent the mean±SEM for each group (n=5). *p<0.05 for AdmMAp44 in comparison to treatment with AdGFP. Black arrows show the injection time of AdmMAp44 and AdGFP.

FIGS. 8A-8C. In vivo transduction and expression efficiency of AdmMAp44 or AdhMAp44 assessed by using Western blot analysis for the HA tag on mouse MAp44 in the sera of WT mice before and after the induction of CAIA. Mice were injected in separate studies with AdmMAp44 or AdhMAp44 i.p. and also locally in the right knee joint. After SDS-PAGE and transfer to nitrocellulose, the blots were probed with anti-HA rabbit antibody. The presence of a HA band (˜43-50 kDa) in serum indicates the successful transduction of cells and protein expression in mice treated with AdmMAp44. FIG. 8A. Presence of HA band in serum at day 0 (lane 3), at day 3 (lane 4) and at day 10 (lane 5) after mice were injected i.p. with AdmMAp44 at day −2 (lane 2). FIG. 8B. Presence of HA band in serum at day 3 (lane 4) and at day 10 (lane 5) after mice were injected in the right knee joint with AdmMAp44 at day −5 (lane 2). Serum from a WT mouse with no injection of adenoviral vectors was used as a negative control (lane 1). FIG. 8C. MAp44 bound to MBL in serum after i.p. injection of AdhMAp44 at day −5. MBL-MAp44 complexes were pulled down using mannose-agarose beads, and the presence of the HA tag on human MAp44 in serum at day 0 (lane 4), day 3 (lane 5), and day 10 (lane 6) was detected using rabbit anti-HA antibody. Serum from a WT mouse without mannose-agarose preparation (lane 1) as well as serum from a WT mouse with no injections with Ad vectors (lane 2) were also examined as controls.

FIGS. 9A-9F. Representative IHS in mice with CAIA comparing the in vivo transduction of AdGFP and AdmMAp44 injected i.p. at day −2 and day 0. All mice were sacrificed at day 10 to assay by IHS the presence of GFP by immunofluorescence or the HA tag (sand grain particles) using anti-HA tag antibodies. FIG. 9A. Mice were not injected with AdGFP or AdmMAp44 but only injected two times with PBS. No green fluorescence was seen in the synovium and in the meniscus. FIG. 9C. Mice were injected two times with AdGFP, and green fluorescence protein is clearly visible in the synovium as marked by a white arrow. FIG. 9E. Mice were injected two times with AdmMAp44 and there was no green fluorescence in the synovium. FIG. 9B. Mice were neither injected with AdGFP nor AdmMAp44 but injected two times with PBS. No sand grain particles were seen in the synovium or in the meniniscus. FIG. 9D. Mice were injected two times with AdGFP and no sand grain particles are visible in the synovium marked by a black arrow. FIG. 9F. Mice were injected two times with AdmMAp44 and there were very distinct sand grain particles (HA) present throughout the synovium. The circular area of the synovium has been enhanced in an insert by 4× to more clearly demonstrate sand grain particles of HA stain. Areas of synovium (S) and meniscus (M) are identified. Magnification for all images in left panels is 10×. Scale bar is 0.1 mm (100 μm). Magnification for all images in right panels is 40×. Scale bar is 0.04 mm (40 μm).

FIG. 10A. Diagrams in numeric sequence illustrating the construction of AdhMAp44 intermediate vectors and production of Ad particles. The human MAp44 cDNA was cloned into pENTCMVMAP44 HA vector by Welgen Inc. The HA sequence was used as a tag to follow expression of both human and mouse AdMAp44. The mouse MAp44 gene was cloned and AdmMAp44 constructed in the same manner. FIG. 10B. AdGFP vector was constructed in the same manner. AdGFP was used as a negative control and to examine the efficiency of transduction in various organs.

FIGS. 11A & 11B show clinical disease activity and prevalence in WT with CAIA. WT mice injected with anti-collagen mAb (arthritomab) alone or LPS alone developed none to low levels of disease in contrast mice injected with anti-collagen mAb followed by the LPS which developed severe disease. FIG. 11A. CDA in WT injected with anti-CII mAb/LPS at day 0 and at day 3, anti-CII mab at day 0 or LPS at day 3 FIG. 11B. Prevalence of disease (%) in WT mice injected with anti-CII mAb/LPS, anti-CII mab or LPS. The data represent the mean±SEM based on n=5 for each group. *p<0.05 in comparison with anti-CII or LPS treatments FIGS. 11C & 11D show percent change in weight over the course of separate CAIA experiments. No major effect of AdhMAp44, AdmMAp44, or AdGFP as compared to PBS was found on the body weight of mice during the course of disease. FIG. 11C. Effect on body weight of a HD and LD of AdhMAp44 as compared to AdGFP and PBS injected i.p. FIG. 11D. Effect on body weight of AdmMAp44 as compared to AdGFP injected i.p. Data are shown as a percent (%) of starting body weight (Mean+SEM). Black arrows in each graph show the injection time of AdhMAp44, AdmMAp44, AdGFP or PBS.

FIGS. 12A-12H. Representative histopathology and C3 deposition images from the knee joints of mice injected i.p. with AdmMAp44 or AdGFP followed by injection of anti-CII mAb and LPS. The top two panels from left to right (FIGS. 12A & 12C) show staining with toluidine-blue from the knee joints of WT mice treated with AdGFP (left panel) and AdmMAp44 (right panel). The second two panels from left to right (FIGS. 12B & 12D) show staining with Toluidine-blue from the ankle joints of WT mice treated with AdGFP (left panel) and AdmMAp44 (right panel). The third set of two panels from left to right (FIGS. 12E & 12G) show staining with anti-C3 Ab from the knee joints of WT mice treated with AdGFP (left panel) and AdmMAp44 (left panel). The fourth set of two panels from left to right (FIGS. 12F & 12H) show staining with anti-C3 Ab (brown color) from the ankle joints of WT mice treated with AdGFP (left panel) and AdmMAp44 (left panel). Areas of synovium (S-black arrow), cartilage (C-black arrow), bone (B) and meniscus (M) are identified. Magnification for all knee joint and ankle joint images shown in FIG. 12 is 20×. Scale bar is 0.1 mm (100 μm).

FIGS. 13A-13B. Effect of AdhMAp44 on RRV-induced arthritis. WT mice injected in the left rear footpad with AdGFP or AdhMAp44 at days −3, 0, and 0. The data shown are derived from the indicated days after the RRV injection in the right footpad. FIG. 13A. CDA over the duration of the experiment. FIG. 13B. Change in weight (%) over the duration of the experiment. The data represent the mean±SEM based on n=4 for each group.

FIG. 14. Mouse models of arthritis. Schematics depicting animal protocols for collagen antibody-induced arthritis and collagen-induced arthritis.

FIG. 15. Collagen antibody-induced arthritis. Severe arthritis develops only in animals injected with anti-CII antibodies followed by injection of LPS.

FIGS. 16A-16B. Effect of natural antibodies C2 and B4 on sub-maximally induced CAIA. FIG. 16A. CDA over the duration of the experiment. FIG. 16B. Prevalence of arthritis (%) over the duration of the experiment. The data represent the mean±SEM for each group. Arrows indicate the injection days of indicated IgM or PBS. *p<0.05.

FIGS. 17A-17F. Representative images of paws from CAIA mice. FIGS. 17A & 17B. Images of paws from CAIA mice at day 3 after LPS injection. FIGS. 17C-17F. Images of paws from CAIA mice at day 10 after LPS injection. FIG. 17A PBS; FIG. 17D control F632 antibody (IgM); FIG. 17E B4IgM; and FIG. 17F C2 IgM.

FIGS. 18A-18B A significant decrease in the clinical disease activity of anti-collagen mAb-induced arthritis in MASP-2/sMAp-% mice compared with WT mice. WT and MASP-2/sMAp^{-/-} mice were injected with 8 mg/mouse/i.p. of ArthritoMab at day 0. All mice were injected with 50 μg/mouse/i.p. with LPS (E. coli strain, 0111B4). All mice were sacrificed at day 10. FIG. 18A. Clinical disease activity (CDA) over the duration of the experiment. There was a 70% decrease in the CDA at day 10 in MASP-2/sMAp^{-/-} mice compared with the WT mice. FIG. 18B. Prevalence of arthritis (%) over the duration of the experiment. Prevalence of disease was 100% in WT and MASP-2/sMAp^{-/-} mice at day 10. The data are expressed as mean of disease±SEM (n=5 each time point). *p<0.05 in comparison with WT mice.

### DETAILED DESCRIPTION OF THE INVENTION

The present application provides MAp44 polypeptides, novel fragments thereof, and uses of these MAp44 polypeptides and fragments thereof for treating complement-mediated diseases, such as arthritis. The present application is based, in part, on the discovery of an essential role of the lectin pathway, and MAp44 in particular, in collagen antibody-induced arthritis disease models. Prior to the present application, the role of LP, and MAp44 in particular, in mediating inflammatory diseases was unclear. For example, studies in mice deficient in different components of the complement system have shown that the AP is both necessary and sufficient to mediate CAIA as neither the LP nor the CP appeared to be required. Studies of LP using MBL-A/C and C4 deficient mice (Banda et al., 2006, J. Immunol. 177: 1904-1912; Ji et al., 2002, Immunity 16: 157-168; Banda et al., 2007, J. Immunol. 179: 4101-4109) showed that disease was largely unaffected by the LP.

The present application has demonstrated that adenoviral-mediated expression of MAp44 dramatically attenuates CAIA and reduces the severity of RRV-induced arthritis in mice, which suggests that the LP is important in the development of tissue injury in these models. The present application provides effective treatment methods based on use of MAp44 and fragments thereof, including, but not limited to, methods involving targeted delivery of MAp44.

The present application thus in one aspect provides constructs comprising MAp44 or novel fragments thereof, optionally linked to a targeting moiety (such as an antibody or fragment thereof).

In another aspect, there are provided methods of treating complement-mediated diseases in an individual, comprising administering to the individual an effective amount of MAp44 or novel fragments thereof, optionally linked to a targeting moiety (such as an antibody or fragment thereof).

Also provided are unit dosage forms, kits, and articles of manufacture that are useful for methods described herein.

### Definitions

The term “individual” refers to a mammal, including humans. An individual includes, but is not limited to, human, bovine, horse, feline, canine, rodent, or primate. In some embodiments, the individual is human.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of the disease. The methods of the invention contemplate any one or more of these aspects of treatment.

The term “effective amount” used herein refers to an amount of a compound or composition sufficient to treat a specified disorder, condition or disease, such as to ameliorate, palliate, lessen, and/or delay one or more of its symptoms.

As used herein, by “pharmaceutically acceptable” or “pharmacologically compatible” is meant a material that is not biologically or otherwise undesirable, e.g., the material may be incorporated into a pharmaceutical composition administered to an individual or patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

As used herein and in the appended claims, the singular forms “a,” “an,” and “the” include plural reference unless the context clearly indicates otherwise.

Reference to “about” a value or parameter herein includes (and describes) embodiments that are directed to that value or parameter per se. For example, description referring to “about X” includes description of “X.”

It is understood that aspects and embodiments of the invention described herein include “consisting” and/or “consisting essentially of” aspects and embodiments.

### MAp44 Polypeptides and Fragments Thereof

The constructs described herein comprise a MAp44 polypeptide or fragment thereof, wherein the MAp44 polypeptide or fragment thereof functions as an inhibitor of complement activity. MAp44, which is present in low levels in serum compared to other MASP proteins such as MASP-1 and MASP-3, functions as a local lectin pathway-specific complement inhibitor. Skjodt et al., Molecular Immunology, 47:2229-30 (2010).

The reduction in complement activity may be incremental (e.g., a 10%, 20%, 30%, 40%, 50%, 60%, 70%, 80%, or 90% reduction in activity) or complete. For example, in some embodiments, a MAp44 polypeptide or fragment thereof can inhibit complement activity by at least 10% (e.g., at least 15%, 20%, 25%, 30%, 35%, 40%, 45%, 50%, 55%, 60%, 65%, 70%, 75%, 80%, 85%, 90%, or 95% or greater) in a standard in vitro red blood cell hemolysis assay, an in vitro CH50eq assay, or the Wieslab® Complement assay (Euro Diagnostica). See, e.g., Kabat and Mayer (eds), “Experimental Immunochemistry, 2nd Edition,” 135-240, Springfield, IL, CC Thomas (1961), pages 135-139, or a conventional variation of that assay such as the chicken erythrocyte hemolysis method as described in, e.g., Hillmen et al. (2004) N Engl J Med 350(6):552.

The CH50eq assay is a method for measuring the total classical complement activity in serum. This test is a lytic assay, which uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50eq assay provides an indirect measure of terminal complement complex (TCC) formation, since the TCC themselves are directly responsible for the hemolysis that is measured.

The assay is well known and commonly practiced by those of skill in the art. Briefly, to activate the classical complement pathway, undiluted serum samples (e.g., human serum samples) are added to microassay wells containing the antibody-sensitized erythrocytes to thereby generate TCC. Next, the activated sera are diluted in microassay wells, which are coated with a capture reagent (e.g., an antibody that binds to one or more components of the TCC). The TCC present in the activated samples bind to the monoclonal antibodies coating the surface of the microassay wells. The wells are washed and, to each well, is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL).

The Wieslab® Complement assay is a commercial kit that can be used to determine specific activation of the CP, AP or LP in serum samples. Briefly, serum is diluted in a solution that blocks the complement pathways that are not being assayed and then incubated in wells coated with activators of the specific pathway being assayed.

An assay useful for detecting the effect of a putative inhibitor on activation of the LP is described in Degn et al., 2009, J. Immunol. 183: 7371-7378. Briefly, putative inhibitors of the LP are incubated with MBL, after which MASP-2 is added and the mixtures transferred to mannan-coated wells. The wells are washed and human complement C4 is added and allowed to incubate. Deposition of C4 fragments can be detected by adding biotin-labeled anti-C4 antibodies followed by streptavidin conjugated to a detectable marker (e.g. radiolabel or fluorescent tag).

Additional methods for detecting and/or measuring complement activity in vitro are set forth and exemplified in the working examples.

The present application in one aspect provides a construct (or a composition comprising the construct such as a pharmaceutical composition, or a vehicle for introducing into an individual an exogenous nucleic acid comprising a sequence for expression of the construct), wherein the construct comprises a MAp44 polypeptide (SEQ ID NO: 44) or fragment thereof. In some embodiments, the MAp44 polypeptide or fragment thereof is between about 50 and about 100 amino acids in length, between about 100 and about 150 amino acids in length, between about 150 and about 200 amino acids in length, between about 200 and about 250 amino acids in length, between about 250 and about 300 amino acids in length, between about 300 and about 350 amino acids in length, or between about 350 and about 380 amino acids in length, and comprises a continuous sequence found in SEQ ID NO: 44. In some embodiments, the MAp44 polypeptide or fragment thereof comprises amino acids 1-137, amino acids 1-176, amino acids 1-296, or amino acids 1-363 of SEQ ID NO: 44. In some embodiments, the MAp44 polypeptide or fragment thereof is fewer than about 100 amino acids in length, fewer than about 200 amino acids in length, fewer than about 250 amino acids in length, or fewer than about 300 amino acids in length. In some embodiments, the MAp44 polypeptide or fragment thereof comprises one or more MAp44 domains selected from the group consisting of CUB1, EGF, CUB2 and CCP1. For example, in some embodiments, the MAp44 polypeptide or fragment thereof comprises one or more sequences selected from the group consisting of SEQ ID NOs: 46, 48, 50 and 52.

In some embodiments, the MAp44 polypeptide or fragment thereof is at least about 50%, 60%, 70%, 80%, 90%, 95%, or 99% homologous to SEQ ID NO: 44.

### Targeting Moieties

The constructs described herein in some embodiments further comprise a targeting moiety. In some embodiments, the targeting moiety is an antibody (such as an antibody recognizing a neoeptitope at a targeted disease site). In some embodiments the targeting moiety is a fragment of complement receptor type 2 (CR2), or a molecule that acts in a similar manner, directing the construct to sites of complement activation. PCT Patent Application No. WO 2004/045520 provides exemplary CR2-based targeting moieties and is specifically incorporated herein by reference. In other embodiments, the targeting moiety is a peptide or other molecule that directs the construct to sites of inflammation, ischemia or oxidative or other forms of injury. In some embodiments, the targeting moiety is a carbohydrate.

In some embodiments, the targeting moiety is an antibody or fragment thereof that specifically binds to Annexin IV or a phospholipid.

Annexin IV belongs to a family of calcium-dependent phospholipid binding proteins. The structure of annexins consists of a conserved Ca²⁺ and membrane binding core of four annexin repeats (eight for annexin IV) and variable N-terminal regions. Annexins are soluble cytosolic proteins, but despite the lack of obvious signal sequences and the apparent inability to enter the classical secretory pathway, annexins have been identified in extracellular fluids or associated with the external cell surface through poorly understood binding sites. Annexin IV is predominantly produced by epithelial cells and is also found at high levels in lung, intestine, pancreas, liver, and kidney. Rescher et al., J. Cell Sci., (2004), 117:2631-2639 and Zernii et al., Biochemistry (Mosc)., (2003), 68(1):129-60.

In some embodiments, the Annexin IV is present on the surface of a cell (and/or in a pathological structure) in an individual that is in or adjacent to a tissue undergoing (or at risk of undergoing) tissue injury. In some embodiments, the Annexin IV is present on the surface of a cell of an individual that is in or adjacent to a tissue undergoing (or at risk of undergoing) oxidative damage. In some embodiments, the Annexin IV is present on the surface of a cell of an individual that is in or adjacent to a tissue undergoing (or at risk of undergoing) ischemia-reperfusion injury. In some embodiments, the Annexin IV is produced by a nucleated cell (such as a mammalian cell). In some embodiments, the Annexin IV is a recombinant protein.

In some embodiments, the epitope on Annexin IV for the antibody or fragment thereof is present on the surface of a cell (and/or in a pathological structure) in an individual that is in or adjacent to a tissue undergoing (or at risk of undergoing) tissue injury but not on the surface of a cell that is in or adjacent to a tissue not undergoing (or is not at risk of undergoing) tissue injury. In some embodiments, the epitope on Annexin IV for the antibody or fragment thereof is present on the surface of a cell that is in or adjacent to a tissue undergoing (or at risk of undergoing) oxidative damage but not on the surface of a cell that is in or adjacent to a tissue not undergoing (or is not at risk of undergoing) oxidative damage. In some embodiments, the epitope on Annexin IV for the antibody or fragment thereof is present on the surface of a cell (and/or in a pathological structure) in an individual that is in or adjacent to a tissue undergoing (or at risk of undergoing) ischemia-reperfusion injury but is not present on the surface of a cell that is in or adjacent to a tissue not undergoing (or is not at risk of undergoing) ischemia reperfusion injury.

In some embodiments, the antibody or fragment thereof specifically binds to a phospholipid, which includes, but is not limited to, phosphatidylethanolamine (PE), cardiolipin (CL), phosphatidylcholine (PC), and in the context of the present application is also intended to encompass sphingolipids and malondialdehyde (MDA). PE, CL, and PC are classes of phospholipids found in biological membranes. Phosphatidylcholine is more commonly found in the exoplasmic or outer leaflet of a cell membrane. It is thought to be transported between membranes within the cell by phosphatidylcholine transfer protein (PCTP). The phospholipid is composed of a choline head group and glycerophosphoric acid with a variety of fatty acids, one being a saturated fatty acid and one being an unsaturated fatty acid. PE consists of a combination of glycerol esterified with two fatty acids and phosphoric acid. Whereas the phosphate group is combined with choline in phosphatidylcholine, it is combined with ethanolamine in PE. The two fatty acids may be the same, or different, and are usually in the 1,2 positions (though they can be in the 1,3 positions). Cardiolipin (IUPAC name “1,3-bis(sn-3′-phosphatidyl)-sn-glycerol”) is an important component of the inner mitochondrial membrane, where it constitutes about 20% of the total lipid composition. Cardiolipin (CL) is a kind of diphosphatidylglycerol lipid, in which two phosphatidylglycerols connect with a glycerol backbone in the center to form a dimeric structure. In most animal tissues, cardiolipin contains 18-carbon fatty alkyl chains with 2 unsaturated bonds on each of them. It has been proposed that the (18:2)4 acyl chain configuration is an important structural requirement for the high affinity of CL to inner membrane proteins in mammalian mitochondria. Phospholipid accumulation has been shown in eyes with age-related macular degeneration (Lommatzsch, et al. (2008) Graefes Arch Clin Exp Ophthalmol. 246(6):803-10).

Malondialdehyde (MDA) is generated from reactive oxygen species (ROS), and as such is often assayed in vivo as a bio-marker of oxidative stress. Reactive oxygen species degrade polyunsaturated lipids, forming malondialdehyde. This compound is a reactive aldehyde and is one of the many reactive electrophile species that cause toxic stress in cells and form covalent protein adducts referred to as advanced lipoxidation end-products (ALE). The production of this aldehyde is also used as a biomarker to measure the level of oxidative stress in an organism. MDA modifications have been shown in eyes with age-related macular degeneration and in the mouse laser-induced CNV model of wet AMD (Weissman et al. (2011) Nature. 478(7367):76-81).

In some embodiments, the phospholipid (such as PE, CL, MDA, and/or PC) is present on the surface of a cell (or in a pathological structure, e.g., drusen) in an individual that is in or adjacent to a tissue undergoing (or at risk of undergoing) tissue injury. In some embodiments, the phospholipid (such as PE, CL, MDA, and/or PC) is present on the surface of a cell (or in a pathological structure, e.g., drusen) of an individual that is in or adjacent to a tissue undergoing (or at risk of undergoing) ocular disease. In some embodiments, the phospholipid (such as PE, CL, MDA, and/or PC) is present on the surface of a cell (or in a pathological structure, e.g., drusen) of an individual that is in or adjacent to a tissue undergoing (or at risk of undergoing) oxidative damage. In some embodiments, the phospholipid is neutral. In some embodiments, the phospholipid is positively charged. In some embodiments, the phospholipid (such as PE, CL, MDA, and/or PC) is oxidized.

In some embodiments, the epitope of phospholipid (such as PE, CL, MDA, and/or PC) to which the antibody or fragment thereof binds is present on the surface of a cell or in a pathological structure (e.g., drusen) in an individual that is in or adjacent to a tissue undergoing (or at risk of undergoing) tissue injury but not on the surface of a cell or in a pathological structure (e.g., drusen) that is in or adjacent to a tissue not undergoing (or is not at risk of undergoing) tissue injury. In some embodiments, the epitope of phospholipid (such as PE, CL, MDA, and/or PC) to which the antibody or fragment thereof binds is present on the surface of a cell or in a pathological structure (e.g., drusen) in an individual that is in or adjacent to a tissue undergoing (or at risk of undergoing) ocular disease but not on the surface of a cell or in a pathological structure (e.g., drusen) that is in or adjacent to a tissue not undergoing (or is not at risk of undergoing) ocular disease. In some embodiments, the epitope on phospholipid (such as PE, CL, MDA, and/or PC) to which the antibody or fragment thereof binds is present on the surface of a cell or in a pathological structure (e.g., drusen) that is in or adjacent to a tissue undergoing (or at risk of undergoing) oxidative damage but not on the surface of a cell or in a pathological structure (e.g., drusen) that is in or adjacent to a tissue not undergoing (or is not at risk of undergoing) oxidative damage.

As described herein, a cell (and/or a pathological structure) that is in or adjacent to a particular tissue as described herein includes a cell (and/or a pathological structure, e.g., drusen) that is part of a tissue or organ, or adjacent to (near, directly next to, in the microenvironment of, bordering, flanking, adjoining) a tissue or organ, in which a certain event (such as non-ischemic injury or oxidative damage) is going to occur, is likely to occur, or is beginning to occur. In the case of an adjacent cell, the cell is sufficiently within the microenvironment of the specific tissue or organ such that conditions of oxidative damage and/or inflammation affect the adjacent cell, as well as the specific tissue or organ. Such a cell may display signs of stress, including, but not limited to, the display of “stress proteins” (e.g., heat shock proteins and other proteins associated with a cellular stress response, including annexins) or other molecules on the cell surface (phospholipids, carbohydrate moieties), including the display of abnormal levels of proteins, modified proteins, or other molecules on the cell surface. Such a cell may be undergoing apoptosis or showing signs of apoptosis, such signs including morphological changes in the cell, chromatin condensation, changes in cellular signal transduction protein interactions, changes in intracellular calcium levels, externalization of phospholipids, cell detachment, loss of cell surface structures, etc.

As used herein, the term “selectively binds to” refers to the specific binding of one protein to another protein, to a lipid, or to a carbohydrate moiety (e.g., the binding of an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art, including, but not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry.

According to the present invention, an “epitope” of a given protein or peptide or other molecule is generally defined, with regard to antibodies, as a part of or site on a larger molecule to which an antibody or antigen-binding fragment thereof will bind, and against which an antibody will be produced. The term epitope can be used interchangeably with the term “antigenic determinant”, “antibody binding site”, or “conserved binding surface” of a given protein or antigen. More specifically, an epitope can be defined by both the amino acid residues involved in antibody binding and also by their conformation in three-dimensional space (e.g., a conformational epitope or the conserved binding surface). An epitope can be included in peptides as small as about 4-6 amino acid residues, or can be included in larger segments of a protein, and need not be comprised of contiguous amino acid residues when referring to a three dimensional structure of an epitope, particularly with regard to an antibody-binding epitope. Antibody-binding epitopes are frequently conformational epitopes rather than a sequential epitope (i.e., linear epitope), or in other words, an epitope defined by amino acid residues arrayed in three dimensions on the surface of a protein or polypeptide to which an antibody binds. As mentioned above, the conformational epitope is not comprised of a contiguous sequence of amino acid residues, but instead, the residues are perhaps widely separated in the primary protein sequence, and are brought together to form a binding surface by the way the protein folds in its native conformation in three dimensions.

Competition assays can be performed using standard techniques in the art (e.g., competitive ELISA or other binding assays). For example, competitive inhibitors can be detected and quantitated by their ability to inhibit the binding of an antigen to a known, labeled antibody (e.g., the mAb B4) or to sera or another composition that is known to contain antibodies against the particular antigen (e.g., sera known to contain natural antibodies against the antigen).

According to the present invention, antibodies are characterized in that they comprise immunoglobulin domains and as such, they are members of the immunoglobulin superfamily of proteins. Generally speaking, an antibody molecule comprises two types of chains. One type of chain is referred to as the heavy or H chain and the other is referred to as the light or L chain. The two chains are present in an equimolar ratio, with each antibody molecule typically having two H chains and two L chains. The two H chains are linked together by disulfide bonds and each H chain is linked to a L chain by a disulfide bond. There are only two types of L chains referred to as lambda (λ) and kappa (κ) chains. In contrast, there are five major H chain classes referred to as isotypes. The five classes include immunoglobulin M (IgM or μ), immunoglobulin D (IgD or δ), immunoglobulin G (IgG or λ), immunoglobulin A (IgA or α), and immunoglobulin E (IgE or ε). The distinctive characteristics between such isotypes are defined by the constant domain of the immunoglobulin and are discussed in detail below. Human immunoglobulin molecules comprise nine isotypes, IgM, IgD, IgE, four subclasses of IgG including IgG1 (γ1), IgG2 (γ2), IgG3 (γ3) and IgG4 (γ4), and two subclasses of IgA including IgA1 (α1) and IgA2 (α2). In humans, IgG subclass 3 and IgM are the most potent complement activators (classical complement system), while IgG subclass 1 and to an even lesser extent, 2, are moderate to low activators of the classical complement system. IgG4 subclass does not activate the complement system (classical or alternative). The only human immunoglobulin isotype known to activate the alternative complement system is IgA. In mice, the IgG subclasses are IgG1, IgG2a, IgG2b and IgG3. Murine IgG1 does not activate complement, while IgG2a, IgG2b and IgG3 are complement activators.

Each H or L chain of an immunoglobulin molecule comprises two regions referred to as L chain variable domains (V_{L} domains) and L chain constant domains (CL domains), and H chain variable domains (V_{H} domains) and H chain constant domains (C_{H} domains). A complete C_{H} domain comprises three sub-domains (CH1, CH2, CH3) and a hinge region. Together, one H chain and one L chain can form an arm of an immunoglobulin molecule having an immunoglobulin variable region. A complete immunoglobulin molecule comprises two associated (e.g., di-sulfide linked) arms. Thus, each arm of a whole immunoglobulin comprises a V_{H+L} region, and a C_{H+L} region. As used herein, the term “variable region” or “V region” refers to a V_{H+L} region (also known as an Fv fragment), a V_{L} region or a V_{H} region. Also as used herein, the term “constant region” or “C region” refers to a C_{H+L} region, a CL region or a C_{H} region.

The antigen specificity of an immunoglobulin molecule is conferred by the amino acid sequence of a variable, or V, region. As such, V regions of different immunoglobulin molecules can vary significantly depending upon their antigen specificity. Certain portions of a V region are more conserved than others and are referred to as framework regions (FR regions). In contrast, certain portions of a V region are highly variable and are designated hypervariable regions. When the V_{L} and V_{H} domains pair in an immunoglobulin molecule, the hypervariable regions from each domain associate and create hypervariable loops that form the antigen binding sites (antigen combining sites). Thus, the hypervariable loops determine the specificity of an immunoglobulin and are termed complementarity-determining regions (CDRs) because their surfaces are complementary to antigens.

Both a L chain and H chain V gene segment contain three regions of substantial amino acid sequence variability. Such regions are referred to as L chain CDR1, CDR2 and CDR3, and H chain CDR1, CDR2 and CDR3, respectively. The length of an L chain CDR1 can vary substantially between different V_{L} regions. For example, the length of CDR1 can vary from about 7 amino acids to about 17 amino acids. In contrast, the lengths of L chain CDR2 and CDR3 typically do not vary between different V_{L} regions. The length of a H chain CDR3 can vary substantially between different V_{H} regions. For example, the length of CDR3 can vary from about 1 amino acid to about 20 amino acids. Each H and L chain CDR region is flanked by FR regions.

Limited digestion of an immunoglobulin with a protease may produce two fragments. An antigen binding fragment is referred to as an Fab, an Fab′, or an F(ab′)₂ fragment. A fragment lacking the ability to bind to antigen is referred to as an Fc fragment. A Fab fragment comprises one arm of an immunoglobulin molecule containing a L chain (V_{L}+C_{L} domains) paired with the V_{H} region and a portion of the C_{H} region (CH1 domain). An Fab′ fragment corresponds to an Fab fragment with part of the hinge region attached to the CH1 domain. An F(ab′)₂ fragment corresponds to two Fab′ fragments that are normally covalently linked to each other through a di-sulfide bond, typically in the hinge regions.

Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab′)₂ fragments), as well as genetically-engineered antibodies or antigen binding fragments thereof, including single chain antibodies (e.g., scFv), humanized antibodies, antibodies that can bind to more than one epitope (e.g., bi-specific antibodies), or antibodies that can bind to one or more different antigens (e.g., bi- or multi-specific antibodies), may also be employed in the invention.

In some embodiments, the targeting moiety of the targeting constructs provided herein comprises an antibody. In some embodiments, the targeting moiety is an scFv. In some embodiments, the targeting moiety is an scFv comprising a (i) a light chain variable domain of SEQ ID NO: 13; and/or (ii) a heavy chain variable domain of SEQ ID NO: 15. In some embodiments, the targeting moiety is an scFv comprising (i) a light chain variable domain of SEQ ID NO: 14; and/or (ii) a heavy chain variable domain of SEQ ID NO: 16. In some embodiments, the targeting moiety is an scFv having the sequence of SEQ ID NO: 17. In some embodiments, the targeting moiety is an scFv having the sequence of SEQ ID NO: 18.

In some embodiments, the targeting moiety is an scFv comprising a (i) a light chain variable domain of SEQ ID NO: 34; and/or (ii) a heavy chain variable domain of SEQ ID NO: 36. In some embodiments, the targeting moiety is an scFv comprising (i) a light chain variable domain of SEQ ID NO: 35; and/or (ii) a heavy chain variable domain of SEQ ID NO: 36. In some embodiments, the targeting moiety is an scFv having the sequence of SEQ ID NO: 37. In some embodiments, the targeting moiety is an scFv having the sequence of SEQ ID NO: 38.

In one embodiment, targeting constructs of the present invention include humanized antibodies or fragments thereof (such as a humanized scFv). A humanized antibody or fragment thereof is a molecule having an antigen binding site derived from an immunoglobulin from a non-human species, the remaining immunoglobulin-derived parts of the molecule being derived from a human immunoglobulin. The antigen binding site may comprise either complete variable regions fused to human constant domains or only the complementarity determining regions (CDRs) grafted onto appropriate human framework regions in the variable domains. A humanized antibody or fragment thereof can be produced, for example, by modeling the antibody variable domains, and producing the antibodies using genetic engineering techniques, such as CDR grafting. A description of various techniques for the production of humanized antibodies is found, for example, in Morrison et al. (1984) Proc. Natl. Acad. Sci. USA 81:6851-55; Whittle et al. (1987) Prot. Eng. 1:499-505; Co et al. (1990) J. Immunol. 148:1149-1154; Co et al. (1992) Proc. Natl. Acad. Sci. USA 88:2869-2873; Carter et al. (1992) Proc. Natl. Acad. Sci. USA 89:4285-4289; Routledge et al. (1991) Eur. J. Immunol. 21:2717-2725 and PCT Patent Publication Nos. WO 91/09967; WO 91/09968 and WO 92/113831.

In some embodiments, the antibody or fragment thereof comprises: (i) a light chain variable domain comprising a sequence (e.g., a light chain CDR1 sequence) of SEQ ID NO: 1, a sequence (e.g., a light chain CDR2 sequence) of SEQ ID NO: 2, or a sequence (e.g., a light chain CDR3 sequence) of SEQ ID NO: 3; and/or (ii) a heavy chain variable domain comprising a sequence (e.g., a heavy chain CDR1 sequence) of SEQ ID NO: 4, a sequence (e.g., a heavy chain CDR2 sequence) of SEQ ID NO: 5, or a sequence (e.g., a heavy chain CDR3 sequence) of SEQ ID NO: 6. In some embodiments, the antibody or fragment thereof comprises: (i) a light chain variable domain comprising a sequence (e.g., a light chain CDR1 sequence) of SEQ ID NO: 7, a sequence (e.g., a light chain CDR2 sequence) of SEQ ID NO: 8, or a sequence (e.g., a light chain CDR3 sequence) of SEQ ID NO: 9; and/or (ii) a heavy chain variable domain comprising a sequence (e.g., a heavy chain CDR1 sequence) of SEQ ID NO: 10, a sequence (e.g., a heavy chain CDR2 sequence) of SEQ ID NO: 11, or a sequence (e.g., a heavy chain CDR3 sequence) of SEQ ID NO: 12.

In some embodiments, the antibody or fragment thereof comprises a light chain variable domain comprising a sequence of SEQ ID NO: 1, a sequence of SEQ ID NO: 2, and a sequence of SEQ ID NO: 3. In some embodiments, the antibody or fragment thereof comprises a light chain variable domain comprising a sequence of SEQ ID NO: 7, a sequence of SEQ ID NO: 8, and a sequence of SEQ ID NO: 9.

In some embodiments, the antibody or fragment thereof comprises a heavy chain variable domain comprising a sequence of SEQ ID NO: 4, a sequence of SEQ ID NO: 5, and a sequence of SEQ ID NO: 6. In some embodiments, the antibody or fragment thereof comprises a heavy chain variable domain comprising a sequence of SEQ ID NO: 10, a sequence of SEQ ID NO: 11, and a sequence of SEQ ID NO: 12.

In some embodiments, the antibody or fragment thereof comprises: (i) a light chain variable domain comprising a sequence of SEQ ID NO: 1, a sequence of SEQ ID NO: 2, and a sequence of SEQ ID NO: 3; and (ii) a heavy chain variable domain comprising a sequence of SEQ ID NO: 4, a sequence of SEQ ID NO: 5, and a sequence of SEQ ID NO: 6. In some embodiments, the antibody or fragment thereof comprises: (i) a light chain variable domain comprising a sequence of SEQ ID NO: 7, a sequence of SEQ ID NO: 8, and a sequence of SEQ ID NO: 9; and (ii) a heavy chain variable domain comprising a sequence of SEQ ID NO: 10, a sequence of SEQ ID NO: 11, and a sequence of SEQ ID NO: 12.

In some embodiments, the antibody or fragment thereof comprises: (i) a light chain CDR1 of SEQ ID NO: 1; (ii) a light chain CDR2 of SEQ ID NO: 2; (iii) a light chain CDR3 of SEQ ID NO: 3; (iv) a heavy chain CDR1 of SEQ ID NO: 4; (v) a heavy chain CDR2 of SEQ ID NO: 5; and (vi) a heavy chain CDR3 of SEQ ID NO: 6. In some embodiments, the antibody or fragment thereof comprises: (i) a light chain CDR1 of SEQ ID NO: 7; (ii) a light chain CDR2 of SEQ ID NO: 8; (iii) a light chain CDR3 of SEQ ID NO: 9; (iv) a heavy chain CDR1 of SEQ ID NO: 10; (v) a heavy chain CDR2 of SEQ ID NO: 11; and (vi) a heavy chain CDR3 of SEQ ID NO: 12.

In some embodiments, the antibody or fragment thereof comprises a light chain variable domain of SEQ ID NO: 13. In some embodiments, the antibody or fragment thereof comprises a heavy chain variable domain of SEQ ID NO: 15. In some embodiments, the antibody or fragment thereof comprises a light chain variable domain of SEQ ID NO: 14. In some embodiments, the antibody or fragment thereof comprises a heavy chain variable domain of SEQ ID NO: 16.

In some embodiments, the antibody or fragment thereof comprises: (i) a light chain variable domain of SEQ ID NO: 13; and (ii) a heavy chain variable domain of SEQ ID NO: 15. In some embodiments, the antibody or fragment thereof comprises: (i) a light chain variable domain of SEQ ID NO: 14; and (ii) a heavy chain variable domain of SEQ ID NO: 16.

In some embodiments, the antibody or fragment is an scFv having the sequence of SEQ ID NO: 17. In some embodiments, the antibody or fragment is an scFv having the sequence of SEQ ID NO: 18.

In some embodiments, the antibody or fragment thereof specifically binds to a phospholipid and comprises: (i) a light chain variable domain comprising a sequence (e.g., a light chain CDR1 sequence) of SEQ ID NO: 25, a sequence (e.g., a light chain CDR2 sequence) of SEQ ID NO: 26, or a sequence (e.g., a light chain CDR3 sequence) of SEQ ID NO: 27; and/or (ii) a heavy chain variable domain comprising a sequence (e.g., a heavy chain CDR1 sequence) of SEQ ID NO: 28, a sequence (e.g., a heavy chain CDR2 sequence) of SEQ ID NO: 29, or a sequence (e.g., a heavy chain CDR3 sequence) of SEQ ID NO: 30. In some embodiments, the antibody or fragment thereof specifically binds to a phospholipid and comprises: (i) a light chain variable domain comprising a sequence (e.g., a light chain CDR1 sequence) of SEQ ID NO: 31, a sequence (e.g., a light chain CDR2 sequence) of SEQ ID NO: 32, or a sequence (e.g., a light chain CDR3 sequence) of SEQ ID NO: 33; and/or (ii) a heavy chain variable domain comprising a sequence (e.g., a heavy chain CDR1 sequence) of SEQ ID NO: 28, a sequence (e.g., a heavy chain CDR2 sequence) of SEQ ID NO: 29, or a sequence (e.g., a heavy chain CDR3 sequence) of SEQ ID NO: 30.

In some embodiments, the antibody or fragment thereof specifically binds to a phospholipid and comprises a light chain variable domain comprising a sequence of SEQ ID NO: 25, a sequence of SEQ ID NO: 26, and a sequence of SEQ ID NO: 27. In some embodiments, the antibody or fragment thereof specifically binds to a phospholipid and comprises a light chain variable domain comprising a sequence of SEQ ID NO: 31, a sequence of SEQ ID NO: 32, and a sequence of SEQ ID NO: 33.

In some embodiments, the antibody or fragment thereof specifically binds to a phospholipid and comprises a heavy chain variable domain comprising a sequence of SEQ ID NO: 28, a sequence of SEQ ID NO: 29, and a sequence of SEQ ID NO: 30.

In some embodiments, the antibody or fragment thereof specifically binds to a phospholipid and comprises: (i) a light chain variable domain comprising a sequence of SEQ ID NO: 25, a sequence of SEQ ID NO: 26, and a sequence of SEQ ID NO: 27; and (ii) a heavy chain variable domain comprising a sequence of SEQ ID NO: 28, a sequence of SEQ ID NO: 29, and a sequence of SEQ ID NO: 30. In some embodiments, the antibody or fragment thereof specifically binds to a phospholipid and comprises: (i) a light chain variable domain comprising a sequence of SEQ ID NO: 31, a sequence of SEQ ID NO: 32, and a sequence of SEQ ID NO: 33; and (ii) a heavy chain variable domain comprising a sequence of SEQ ID NO: 28, a sequence of SEQ ID NO: 29, and a sequence of SEQ ID NO: 30.

In some embodiments, the antibody or fragment thereof specifically binds to a phospholipid and comprises: (i) a light chain CDR1 of SEQ ID NO: 25; (ii) a light chain CDR2 of SEQ ID NO: 26; (iii) a light chain CDR3 of SEQ ID NO: 27; (iv) a heavy chain CDR1 of SEQ ID NO: 28; (v) a heavy chain CDR2 of SEQ ID NO: 29; and (vi) a heavy chain CDR3 of SEQ ID NO: 30. In some embodiments, the antibody or fragment thereof specifically binds to a phospholipid and comprises: (i) a light chain CDR1 of SEQ ID NO: 31; (ii) a light chain CDR2 of SEQ ID NO: 32; (iii) a light chain CDR3 of SEQ ID NO: 33; (iv) a heavy chain CDR1 of SEQ ID NO: 28; (v) a heavy chain CDR2 of SEQ ID NO: 29; and (vi) a heavy chain CDR3 of SEQ ID NO: 30.

In some embodiments, the antibody or fragment thereof comprises a light chain variable domain of SEQ ID NO: 34. In some embodiments, the antibody or fragment thereof comprises a heavy chain variable domain of SEQ ID NO: 36. In some embodiments, the antibody or fragment thereof comprises a light chain variable domain of SEQ ID NO: 35.

In some embodiments, the antibody or fragment thereof comprises: (i) a light chain variable domain of SEQ ID NO: 34; and (ii) a heavy chain variable domain of SEQ ID NO: 36. In some embodiments, the antibody or fragment thereof comprises: (i) a light chain variable domain of SEQ ID NO: 35; and (ii) a heavy chain variable domain of SEQ ID NO: 36.

In some embodiments, the antibody or fragment is an scFv having the sequence of SEQ ID NO: 37. In some embodiments, the antibody or fragment is an scFv having the sequence of SEQ ID NO: 38.

In some embodiments, the targeting moiety is a multivalent antibody or fragment thereof. In some embodiments, the targeting moiety is a bi-specific antibody or fragment thereof. In some embodiments, the targeting moiety is a bi-specific antibody or fragment thereof that binds to both Annexin IV and a phospholipid. In some embodiments, the targeting moiety is a bi-specific antibody or fragment thereof, wherein a first arm of the antibody or fragment thereof binds to Annexin IV and a second arm of the antibody or fragment thereof binds to a phospholipid. For example, in some embodiments the targeting moiety is a bi-specific antibody or fragment thereof, wherein (a) a first arm of the antibody or fragment thereof binds to Annexin IV and comprises a sequence described above for antibodies or fragments thereof that bind to Annexin IV; and (b) a second arm of the antibody or fragment thereof binds to a phospholipid and comprises a sequence described above for antibodies or fragments thereof that bind to a phospholipid. For example, in some embodiments the targeting moiety is a bi-specific antibody or fragment thereof, comprising (a) a first arm that is an arm of naturally occurring antibody B4; and (b) a second arm that is an arm of naturally occurring antibody C2.

In some embodiments, there is provided a construct comprising (a) a bi-specific antibody or fragment thereof that binds to both Annexin IV and a phospholipid as described above; and (b) an inhibitor of complement activity (such as a MAp44 polypeptide or fragment thereof). It is to be understood that the construct described herein may be used for any of the methods described in the present invention.

### MAp44 Constructs

The methods described herein comprise administration of MAp44 constructs. The present application further provides novel MAp44 constructs (such as novel MAp44 fragments and/or novel targeting moiety-MAp44 fusion constructs described herein). The MAp44 constructs are described in this section in detail, and any of the constructs described in the section herein can be used for any of the methods described in the present invention.

The present application further provides methods of delivering any of a MAp44 polypeptide or fragment thereof disclosed herein to an individual by administering to the individual any one of the constructs described herein.

The present application further provides methods of delivering any of a MAp44 polypeptide or fragment thereof disclosed herein to a site of complement activation, a site of tissue injury (such as non-ischemic tissue injury), or a site of complement-associated disease in an individual by administering to the individual any one of the constructs described herein.

In some embodiments, there is provided a construct (or a composition comprising the construct such as a pharmaceutical composition, or a vehicle for introducing into an individual an exogenous nucleic acid comprising a sequence for expression of the construct), wherein the construct comprises a MAp44 polypeptide or fragment thereof.

In some embodiments, there is provided a construct (or a composition comprising the construct such as a pharmaceutical composition, or a vehicle for introducing into an individual an exogenous nucleic acid comprising a sequence for expression of the construct), wherein the construct comprises a MAp44 polypeptide or fragment thereof that comprises the sequence of SEQ ID NO: 44. In some embodiments, the MAp44 polypeptide or fragment thereof is between about 50 and about 100 amino acids in length, between about 100 and about 150 amino acids in length, between about 150 and about 200 amino acids in length, between about 200 and about 250 amino acids in length, between about 250 and about 300 amino acids in length, between about 300 and about 350 amino acids in length, or between about 350 and about 380 amino acids in length, and comprises a continuous sequence found in SEQ ID NO: 44. In some embodiments, the MAp44 polypeptide or fragment thereof comprises amino acids 1-137, amino acids 1-176, amino acids 1-296, or amino acids 1-363 of SEQ ID NO: 44. In some embodiments, the MAp44 polypeptide or fragment thereof is fewer than about 100 amino acids in length, fewer than about 200 amino acids in length, fewer than about 250 amino acids in length, or fewer than about 300 amino acids in length. In some embodiments, the MAp44 polypeptide or fragment thereof comprises one or more sequences selected from the group consisting of SEQ ID NOs: 46, 48, 50 and 52.

In some embodiments, the MAp44 polypeptide or fragment thereof is at least about 50%, 60%, 70%, 80%, 90%, 95%, or 99% homologous to SEQ ID NO: 44.

In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof. In some embodiments, the construct is a fusion protein. In some embodiments, the antibody or fragment thereof (hereinafter also referred to as the “targeting moiety”) and the MAp44 polypeptide or fragment thereof are linked via a linker (such as a peptide linker). In some embodiments, the targeting moiety and the MAp44 polypeptide or fragment thereof are directly linked.

In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment thereof comprises: (i) a light chain variable domain comprising a sequence of SEQ ID NO: 1, a sequence of SEQ ID NO: 2, or a sequence of SEQ ID NO: 3; and/or (ii) a heavy chain variable domain comprising a sequence of SEQ ID NO: 4, a sequence of SEQ ID NO: 5, or a sequence of SEQ ID NO: 6. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment thereof comprises: (i) a light chain variable domain comprising a sequence of SEQ ID NO: 7, a sequence of SEQ ID NO: 8, or a sequence of SEQ ID NO: 9; and/or (ii) a heavy chain variable domain comprising a sequence of SEQ ID NO: 10, a sequence of SEQ ID NO: 11, or a sequence of SEQ ID NO: 12. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or at risk of undergoing) tissue injury (such as non-ischemic injury) and/or oxidative damage. In some embodiments, the Annexin IV is produced by a nucleated cell (such as a mammalian cell). In some embodiments, the Annexin IV is a recombinant protein. In some embodiments, the construct is a fusion protein. In some embodiments, the targeting moiety and the MAp44 polypeptide or fragment thereof are linked via a linker (such as a peptide linker). In some embodiments, the targeting moiety and the MAp44 polypeptide or fragment thereof are directly linked.

In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment thereof comprises a light chain variable domain comprising a sequence of SEQ ID NO: 1, a sequence of SEQ ID NO: 2, and a sequence of SEQ ID NO: 3. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment thereof comprises a light chain variable domain comprising a sequence of SEQ ID NO: 7, a sequence of SEQ ID NO: 8, and a sequence of SEQ ID NO: 9. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or at risk of undergoing) tissue injury (such as non-ischemic injury) and/or oxidative damage. In some embodiments, the Annexin IV is produced by a nucleated cell (such as a mammalian cell). In some embodiments, the Annexin IV is a recombinant protein. In some embodiments, the construct is a fusion protein. In some embodiments, the targeting moiety and the MAp44 polypeptide or fragment thereof are linked via a linker (such as a peptide linker). In some embodiments, the targeting moiety and the MAp44 polypeptide or fragment thereof are directly linked.

In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment thereof comprises a heavy chain variable domain comprising a sequence of SEQ ID NO: 4, a sequence of SEQ ID NO: 5, and a sequence of SEQ ID NO: 6. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment thereof comprises a heavy chain variable domain comprising a sequence of SEQ ID NO: 10, a sequence of SEQ ID NO: 11, and a sequence of SEQ ID NO: 12. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or at risk of undergoing) tissue injury (such as non-ischemic injury) and/or oxidative damage. In some embodiments, the Annexin IV is produced by a nucleated cell (such as a mammalian cell). In some embodiments, the Annexin IV is a recombinant protein. In some embodiments, the construct is a fusion protein. In some embodiments, the targeting moiety and the MAp44 polypeptide or fragment thereof are linked via a linker (such as a peptide linker). In some embodiments, the targeting moiety and the MAp44 polypeptide or fragment thereof are directly linked.

In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment thereof comprises: (i) a light chain variable domain comprising a sequence of SEQ ID NO: 1, a sequence of SEQ ID NO: 2, and a sequence of SEQ ID NO: 3; and (ii) a heavy chain variable domain comprising a sequence of SEQ ID NO: 4, a sequence of SEQ ID NO: 5, and a sequence of SEQ ID NO: 6. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment thereof comprises: (i) a light chain variable domain comprising a sequence of SEQ ID NO: 7, a sequence of SEQ ID NO: 8, and a sequence of SEQ ID NO: 9; and (ii) a heavy chain variable domain comprising a sequence of SEQ ID NO: 10, a sequence of SEQ ID NO: 11, and a sequence of SEQ ID NO: 12. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or at risk of undergoing) tissue injury (such as non-ischemic injury) and/or oxidative damage. In some embodiments, the Annexin IV is produced by a nucleated cell (such as a mammalian cell). In some embodiments, the Annexin IV is a recombinant protein. In some embodiments, the construct is a fusion protein. In some embodiments, the targeting moiety and the MAp44 polypeptide or fragment thereof are linked via a linker (such as a peptide linker). In some embodiments, the targeting moiety and the MAp44 polypeptide or fragment thereof are directly linked.

In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment thereof comprises: (i) a light chain CDR1 of SEQ ID NO: 1; (ii) a light chain CDR2 of SEQ ID NO: 2; (iii) a light chain CDR3 of SEQ ID NO: 3; (iv) a heavy chain CDR1 of SEQ ID NO: 4; (v) a heavy chain CDR2 of SEQ ID NO: 5; and (vi) a heavy chain CDR3 of SEQ ID NO: 6. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment thereof comprises: (i) a light chain CDR1 of SEQ ID NO: 7; (ii) a light chain CDR2 of SEQ ID NO: 8; (iii) a light chain CDR3 of SEQ ID NO: 9; (iv) a heavy chain CDR1 of SEQ ID NO: 10; (v) a heavy chain CDR2 of SEQ ID NO: 11; and (vi) a heavy chain CDR3 of SEQ ID NO: 12. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or at risk of undergoing) tissue injury (such as non-ischemic injury) and/or oxidative damage. In some embodiments, the Annexin IV is produced by a nucleated cell (such as a mammalian cell). In some embodiments, the Annexin IV is a recombinant protein. In some embodiments, the construct is a fusion protein. In some embodiments, the targeting moiety and the MAp44 polypeptide or fragment thereof are linked via a linker (such as a peptide linker). In some embodiments, the targeting moiety and the MAp44 polypeptide or fragment thereof are directly linked.

In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment thereof comprises a light chain variable domain of SEQ ID NO: 13. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment thereof comprises a heavy chain variable domain of SEQ ID NO: 15. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment thereof comprises a light chain variable domain of SEQ ID NO: 14. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment thereof comprises a heavy chain variable domain of SEQ ID NO: 16. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or at risk of undergoing) tissue injury (such as non-ischemic injury) and/or oxidative damage. In some embodiments, the Annexin IV is produced by a nucleated cell (such as a mammalian cell). In some embodiments, the Annexin IV is a recombinant protein. In some embodiments, the construct is a fusion protein. In some embodiments, the targeting moiety and the MAp44 polypeptide or fragment thereof are linked via a linker (such as a peptide linker). In some embodiments, the targeting moiety and the MAp44 polypeptide or fragment thereof are directly linked.

In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment thereof comprises: (i) a light chain variable domain of SEQ ID NO: 13; and (ii) a heavy chain variable domain of SEQ ID NO: 15. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment thereof comprises: (i) a light chain variable domain of SEQ ID NO: 14; and (ii) a heavy chain variable domain of SEQ ID NO: 16. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or at risk of undergoing) tissue injury (such as non-ischemic injury) and/or oxidative damage. In some embodiments, the Annexin IV is produced by a nucleated cell (such as a mammalian cell). In some embodiments, the Annexin IV is a recombinant protein. In some embodiments, the construct is a fusion protein. In some embodiments, the targeting moiety and the MAp44 polypeptide or fragment thereof are linked via a linker (such as a peptide linker). In some embodiments, the targeting moiety and the MAp44 polypeptide or fragment thereof are directly linked.

In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment is an scFv having the sequence of SEQ ID NO: 17. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to Annexin IV; and (b) a MAp44 polypeptide or fragment thereof, wherein the antibody or fragment is an scFv having the sequence of SEQ ID NO: 18. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or at risk of undergoing) tissue injury (such as non-ischemic injury) and/or oxidative damage. In some embodiments, the Annexin IV is produced by a nucleated cell (such as a mammalian cell). In some embodiments, the Annexin IV is a recombinant protein. In some embodiments, the construct is a fusion protein. In some embodiments, the targeting moiety and the MAp44 polypeptide or fragment thereof are linked via a linker (such as a peptide linker). In some embodiments, the targeting moiety and the MAp44 polypeptide or fragment thereof are directly linked.

In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to a phospholipid (such as PE, CL, MDA, and/or PC); and (b) a MAp44 polypeptide or fragment thereof, wherein the antibody or fragment thereof comprises: (i) a light chain variable domain comprising a sequence of SEQ ID NO: 25, a sequence of SEQ ID NO: 26, or a sequence of SEQ ID NO: 27; and/or (ii) a heavy chain variable domain comprising a sequence of SEQ ID NO: 28, a sequence of SEQ ID NO: 29, or a sequence of SEQ ID NO: 30. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to a phospholipid (such as PE, CL, MDA, and/or PC); and (b) a MAp44 polypeptide or fragment thereof, wherein the antibody or fragment thereof comprises: (i) a light chain variable domain comprising a sequence of SEQ ID NO: 31, a sequence of SEQ ID NO: 32, or a sequence of SEQ ID NO: 33; and/or (ii) a heavy chain variable domain comprising a sequence of SEQ ID NO: 28, a sequence of SEQ ID NO: 29, or a sequence of SEQ ID NO: 30. In some embodiments, the antibody or fragment thereof competitively inhibits the binding of a pathogenic antibody (such as monoclonal antibody C2) to the phospholipid. In some embodiments, the antibody or antibody fragment thereof binds to the same epitope as a pathogenic antibody (such as monoclonal antibody C2) to the phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the antibody or fragment thereof binds to malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the MAp44 polypeptide or fragment thereof are linked via a linker (such as a peptide linker). In some embodiments, the targeting moiety and the MAp44 polypeptide or fragment thereof are directly linked.

In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to a phospholipid (such as PE, CL, MDA, and/or PC); and (b) a MAp44 polypeptide or fragment thereof, wherein the antibody or fragment thereof comprises a light chain variable domain comprising a sequence of SEQ ID NO: 25, a sequence of SEQ ID NO: 26, and a sequence of SEQ ID NO: 27. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to a phospholipid (such as PE, CL, MDA, and/or PC); and (b) a MAp44 polypeptide or fragment thereof, wherein the antibody or fragment thereof comprises a light chain variable domain comprising a sequence of SEQ ID NO: 31, a sequence of SEQ ID NO: 32, and a sequence of SEQ ID NO: 33. In some embodiments, the antibody or fragment thereof competitively inhibits the binding of a pathogenic antibody (such as monoclonal antibody C2) to the phospholipid. In some embodiments, the antibody or antibody fragment thereof binds to the same epitope as a pathogenic antibody (such as monoclonal antibody C2) to the phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the antibody or fragment thereof binds to malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the MAp44 polypeptide or fragment thereof are linked via a linker (such as a peptide linker). In some embodiments, the targeting moiety and the MAp44 polypeptide or fragment thereof are directly linked.

In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to a phospholipid (such as PE, CL, MDA, and/or PC); and (b) a MAp44 polypeptide or fragment thereof, wherein the antibody or fragment thereof comprises a heavy chain variable domain comprising a sequence of SEQ ID NO: 28, a sequence of SEQ ID NO: 29, and a sequence of SEQ ID NO: 30. In some embodiments, the antibody or fragment thereof competitively inhibits the binding of a pathogenic antibody (such as monoclonal antibody C2) to the phospholipid. In some embodiments, the antibody or antibody fragment thereof binds to the same epitope as a pathogenic antibody (such as monoclonal antibody C2) to the phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the antibody or fragment thereof binds to malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the MAp44 polypeptide or fragment thereof are linked via a linker (such as a peptide linker). In some embodiments, the targeting moiety and the MAp44 polypeptide or fragment thereof are directly linked.

In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to a phospholipid (such as PE, CL, MDA, and/or PC); and (b) a MAp44 polypeptide or fragment thereof, wherein the antibody or fragment thereof comprises: (i) a light chain variable domain comprising a sequence of SEQ ID NO: 25, a sequence of SEQ ID NO: 26, and a sequence of SEQ ID NO: 27; and (ii) a heavy chain variable domain comprising a sequence of SEQ ID NO: 28, a sequence of SEQ ID NO: 29, and a sequence of SEQ ID NO: 30. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to a phospholipid (such as PE, CL, MDA, and/or PC); and (b) a MAp44 polypeptide or fragment thereof, wherein the antibody or fragment thereof comprises: (i) a light chain variable domain comprising a sequence of SEQ ID NO: 31, a sequence of SEQ ID NO: 32, and a sequence of SEQ ID NO: 33; and (ii) a heavy chain variable domain comprising a sequence of SEQ ID NO: 28, a sequence of SEQ ID NO: 29, and a sequence of SEQ ID NO: 30. In some embodiments, the antibody or fragment thereof competitively inhibits the binding of a pathogenic antibody (such as monoclonal antibody C2) to the phospholipid. In some embodiments, the antibody or antibody fragment thereof binds to the same epitope as a pathogenic antibody (such as monoclonal antibody C2) to the phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the antibody or fragment thereof binds to malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the MAp44 polypeptide or fragment thereof are linked via a linker (such as a peptide linker). In some embodiments, the targeting moiety and the MAp44 polypeptide or fragment thereof are directly linked.

In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to a phospholipid (such as PE, CL, MDA, and/or PC); and (b) a MAp44 polypeptide or fragment thereof, wherein the antibody or fragment thereof comprises: (i) a light chain CDR1 of SEQ ID NO: 25; (ii) a light chain CDR2 of SEQ ID NO: 26; (iii) a light chain CDR3 of SEQ ID NO: 27; (iv) a heavy chain CDR1 of SEQ ID NO: 28; (v) a heavy chain CDR2 of SEQ ID NO: 29; and (vi) a heavy chain CDR3 of SEQ ID NO: 30. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to a phospholipid (such as PE, CL, MDA, and/or PC); and (b) a MAp44 polypeptide or fragment thereof, wherein the antibody or fragment thereof comprises: (i) a light chain CDR1 of SEQ ID NO: 31; (ii) a light chain CDR2 of SEQ ID NO: 32; (iii) a light chain CDR3 of SEQ ID NO: 33; (iv) a heavy chain CDR1 of SEQ ID NO: 28; (v) a heavy chain CDR2 of SEQ ID NO: 29; and (vi) a heavy chain CDR3 of SEQ ID NO: 30. In some embodiments, the antibody or fragment thereof competitively inhibits the binding of a pathogenic antibody (such as monoclonal antibody C2) to the phospholipid. In some embodiments, the antibody or antibody fragment thereof binds to the same epitope as a pathogenic antibody (such as monoclonal antibody C2) to the phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the antibody or fragment thereof binds to malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the MAp44 polypeptide or fragment thereof are linked via a linker (such as a peptide linker). In some embodiments, the targeting moiety and the MAp44 polypeptide or fragment thereof are directly linked.

In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to a phospholipid (such as PE, CL, MDA, and/or PC); and (b) a MAp44 polypeptide or fragment thereof, wherein the antibody or fragment thereof comprises a light chain variable domain of SEQ ID NO: 34. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to a phospholipid (such as PE, CL, MDA, and/or PC); and (b) a MAp44 polypeptide or fragment thereof, wherein the antibody or fragment thereof comprises a heavy chain variable domain of SEQ ID NO: 36. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to a phospholipid (such as PE, CL, MDA, and/or PC); and (b) a MAp44 polypeptide or fragment thereof, wherein the antibody or fragment thereof comprises a light chain variable domain of SEQ ID NO: 35. In some embodiments, the antibody or fragment thereof competitively inhibits the binding of a pathogenic antibody (such as monoclonal antibody C2) to the phospholipid. In some embodiments, the antibody or antibody fragment thereof binds to the same epitope as a pathogenic antibody (such as monoclonal antibody C2) to the phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the antibody or fragment thereof binds to malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the MAp44 polypeptide or fragment thereof are linked via a linker (such as a peptide linker). In some embodiments, the targeting moiety and the MAp44 polypeptide or fragment thereof are directly linked.

In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to a phospholipid (such as PE, CL, MDA, and/or PC); and (b) a MAp44 polypeptide or fragment thereof, wherein the antibody or fragment thereof comprises: (i) a light chain variable domain of SEQ ID NO: 34; and (ii) a heavy chain variable domain of SEQ ID NO: 36. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to a phospholipid (such as PE, CL, MDA, and/or PC); and (b) a MAp44 polypeptide or fragment thereof, wherein the antibody or fragment thereof comprises: (i) a light chain variable domain of SEQ ID NO: 35; and (ii) a heavy chain variable domain of SEQ ID NO: 36. In some embodiments, the antibody or fragment thereof competitively inhibits the binding of a pathogenic antibody (such as monoclonal antibody C2) to the phospholipid. In some embodiments, the antibody or antibody fragment thereof binds to the same epitope as a pathogenic antibody (such as monoclonal antibody C2) to the phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the antibody or fragment thereof binds to malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the MAp44 polypeptide or fragment thereof are linked via a linker (such as a peptide linker). In some embodiments, the targeting moiety and the MAp44 polypeptide or fragment thereof are directly linked.

In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to a phospholipid (such as PE, CL, MDA, and/or PC); and (b) a MAp44 polypeptide or fragment thereof, wherein the antibody or fragment is an scFv having the sequence of SEQ ID NO: 37. In some embodiments, there is provided a construct (or a composition comprising the construct such as a pharmaceutical composition, or a vehicle for introducing into an individual an exogenous nucleic acid comprising a sequence for expression of the construct), wherein the construct comprises (a) an antibody or fragment thereof, wherein the antibody or fragment thereof specifically binds to a phospholipid (such as PE, CL, MDA, and/or PC); and (b) a MAp44 polypeptide or fragment thereof, wherein the antibody or fragment is an scFv having the sequence of SEQ ID NO: 38. In some embodiments, the antibody or fragment thereof competitively inhibits the binding of a pathogenic antibody (such as monoclonal antibody C2) to the phospholipid. In some embodiments, the antibody or antibody fragment thereof binds to the same epitope as a pathogenic antibody (such as monoclonal antibody C2) to the phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the antibody or fragment thereof binds to malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the MAp44 polypeptide or fragment thereof are linked via a linker (such as a peptide linker). In some embodiments, the targeting moiety and the MAp44 polypeptide or fragment thereof are directly linked.

In some embodiments, the targeting moiety is a multivalent antibody or fragment thereof. In some embodiments, the targeting moiety is a bi-specific antibody or fragment thereof. In some embodiments, the targeting moiety is a bi-specific antibody or fragment thereof that binds to both Annexin IV and a phospholipid. In some embodiments, the targeting moiety is a bi-specific antibody or fragment thereof, wherein a first arm of the antibody or fragment thereof binds to Annexin IV and a second arm of the antibody or fragment thereof binds to a phospholipid. For example, in some embodiments the targeting moiety is a bi-specific antibody or fragment thereof, wherein (a) a first arm of the antibody or fragment thereof binds to Annexin IV and comprises a sequence described above for antibodies or fragments thereof that bind to Annexin IV; and (b) a second arm of the antibody or fragment thereof binds to a phospholipid and comprises a sequence described above for antibodies or fragments thereof that bind to a phospholipid. For example, in some embodiments the targeting moiety is a bi-specific antibody or fragment thereof, comprising (a) a first arm that is an arm of naturally occurring antibody B4; and (b) a second arm that is an arm of naturally occurring antibody C2.

In some embodiments, the targeting moiety and the MAp44 polypeptide or fragment thereof are directly bonded, covalently bonded, or, reversibly bonded.

A “targeting construct” used herein refers to a non-naturally occurring molecule comprising a “targeting moiety” and a MAp44 polypeptide or fragment thereof. The targeting moiety is capable of specifically binding to Annexin IV or a phospholipid. The targeting moiety of the targeting construct is responsible for targeted delivery of the molecule to the sites of, e.g., complement activation. The MAp44 polypeptide or fragment thereof is responsible for therapeutic activity, e.g., specifically inhibiting complement activation. The targeting moiety and the MAp44 polypeptide or fragment thereof of a targeting construct molecule can be linked together by any methods known in the art, as long as the desired functionalities of the two portions are maintained.

The targeting construct described herein thus generally has the dual functions of binding to an epitope recognized by an antibody described herein and exerting therapeutic activity. An “epitope of monoclonal C2 antibody or B4 antibody” refers to any molecule that binds to a naturally occurring C2 or B4 antibody, which includes epitopes that bind to a C2 or B4 antibody with a binding affinity that is about any of 10%, 20%, 30%, 40%, 50%, 60%, 70%, 80%, 90%, or 100% of the epitope that naturally binds a C2 or B4 antibody. Binding affinity can be determined by any method known in the art, including for example, surface plasmon resonance, calorimetry titration, ELISA, and flow cytometry.

In some embodiments, a targeting construct described herein is generally capable of inhibiting complement activation (for example inhibiting activation of the lectin pathway). The targeting construct may be a more potent complement inhibitor than a MAp44 polypeptide or fragment thereof as described herein. For example, in some embodiments, the targeting construct has a complement inhibitory activity that is about any of 1.5, 2, 2.5, 3, 3.5, 4, 5, 6, 7, 8, 9, 10, 12, 14, 16, 18, 20, 25, 30, 40, or more times that of a MAp44 polypeptide or fragment thereof as described herein. In some embodiments, the targeting construct has an EC50 of less than about any of 100 nM, 90 nM, 80 nM, 70 nM, 60 nM, 50 nM, 40 nM, 30 nM, 20 nM, or 10 nM, inclusive, including any values in between these numbers. In some embodiments, the targeting construct has an EC50 of about 5 to 60 nM, including for example any of 8 to 50 nM, 8 to 20 nM, 10 to 40 nM, and 20 to 30 nM. In some embodiments, the targeting construct has complement inhibitory activity that is about any of 50%, 60%, 70%, 80%, 90%, or 100% of that of a MAp44 polypeptide or fragment thereof as described herein.

Complement inhibition can be evaluated based on any methods known in the art, including for example, in vitro zymosan assays, assays for lysis of erythrocytes, antibody or immune complex activation assays, alternative pathway activation assays, and mannan activation assays.

In some embodiments, the targeting construct is a fusion protein. “Fusion protein” used herein refers to two or more peptides, polypeptides, or proteins operably linked to each other. In some embodiments, the targeting moiety and the MAp44 polypeptide or fragment thereof are directly fused to each other. In some embodiments, the targeting moiety and the MAp44 polypeptide or fragment thereof are linked by an amino acid linker sequence. Examples of linker sequences are known in the art, and include, for example, (Gly₄Ser), (Gly₄Ser)₂, (Gly₄Ser)₃, (Gly₃Ser)₄, (SerGly₄), (SerGly₄)₂, (SerGly₄)₃, and (SerGly₄)₄. Linking sequences can also comprise “natural” linking sequences found between different domains of complement factors. The order of targeting moiety and MAp44 polypeptide or fragment thereof in the fusion protein can vary. For example, in some embodiments, the C-terminus of the targeting moiety is fused (directly or indirectly) to the N-terminus of the MAp44 polypeptide or fragment thereof of the targeting construct. In some embodiments, the N-terminus of the targeting moiety is fused (directly or indirectly) to the C-terminus of the MAp44 polypeptide or fragment thereof of the targeting construct.

In some embodiments, the targeting moiety of a targeting construct is encoded by a polynucleotide comprising a nucleic acid sequence of any one of SEQ ID NOs: 19-24 and 57. In some embodiments, the targeting construct molecule is encoded by a polynucleotide comprising a nucleic acid sequence that is at least about 50%, 60%, 70%, 80%, 90%, 91%, 92%, 93%, 94%, 95%, 96%, 97%, 98%, or 99% identical to that of any of SEQ ID NOs: 19-24 and 57.

In some embodiments, the targeting construct comprises a targeting moiety and a MAp44 polypeptide or fragment thereof linked via a chemical cross-linker. Linking of the two portions can occur on reactive groups located on the two moieties. Reactive groups that can be targeted using a crosslinker include primary amines, sulfhydryls, carbonyls, carbohydrates, and carboxylic acids, or active groups that can be added to proteins. Examples of chemical linkers are well known in the art and include, but are not limited to, bismaleimidohexane, maleimidobenzoyl-N-hydroxysuccinimide ester, NHS-Esters-Maleimide Crosslinkers such as SPDP, carbodiimide, glutaraldehyde, MBS, Sulfo-MBS, SMPB, sulfo-SMPB, GMBS, Sulfo-GMBS, EMCS, Sulfo-EMCS, imidoester crosslinkers such as DMA, DMP, DMS, DTBP, EDC and DTME.

In some embodiments, the targeting moiety and the MAp44 polypeptide or fragment thereof are non-covalently linked. For example, the two portions may be brought together by two interacting bridging proteins (such as biotin and streptavidin), each linked to a targeting moiety or a MAp44 polypeptide or fragment thereof.

In some embodiments, the targeting moiety of the targeting construct is joined (e.g., directly or by way of a linker) to the amino-terminus of the MAp44 polypeptide or fragment thereof. In some embodiments, the targeting moiety of the targeting construct is joined (e.g., directly or by way of a linker) to the carboxy-terminus of the MAp44 polypeptide or fragment thereof.

In some embodiments, the light chain of the targeting moiety of the targeting construct is linked to at least one MAp44 polypeptide or fragment thereof and the heavy chain is linked to at least one MAp44 polypeptide or fragment thereof. The two or more MAp44 polypeptides or fragments thereof can be the same or different. For example, in some embodiments, the targeting construct comprises the Fab fragment of a targeting moiety described herein, wherein: (i) the light chain of the Fab fragment is linked to (at its C-terminal end) a MAp44 polypeptide or fragment thereof described herein; and (ii) the heavy chain of the Fab fragment is linked to (at its C-terminal end) the same or a different MAp44 polypeptide or fragment thereof described herein. Appropriate pairing of the two chains can be expected to occur as an inherent property of the Fab. The MAp44 polypeptide or fragment thereof and the light chain or heavy chain of the Fab can be joined together directly or by way of a linker sequence (such as any of those described herein).

In some embodiments, the targeting construct comprises two or more (same or different) targeting moieties described herein. In some embodiments, the targeting construct comprises two or more (same or different) MAp44 polypeptides or fragments thereof described herein. These two or more targeting moieties or MAp44 polypeptides or fragments thereof may be tandemly linked (such as fused) to each other. In some embodiments, the targeting construct comprises a targeting moiety and two or more (such as three, four, five, or more) MAp44 polypeptides or fragments thereof. In some embodiments, the targeting construct comprises a MAp44 polypeptide or fragment thereof and two or more (such as three, four, five, or more) targeting moieties. In some embodiments, the targeting construct comprises two or more targeting moieties and two or more MAp44 polypeptides or fragments thereof.

In some embodiments, there is provided an isolated construct. In some embodiments, the constructs form dimers or multimers.

The MAp44 polypeptide or fragment thereof and the targeting moiety in the targeting construct can be from the same species (such as human or mouse), or from different species.

### Variants of Constructs

Also encompassed are variants of the constructs. A variant of the construct described herein may be: (i) one in which one or more of the amino acid residues of the targeting moiety and/or the MAp44 polypeptide or fragment thereof are substituted with a conserved or non-conserved amino acid residue (preferably a conserved amino acid residue) and such substituted amino acid residue may or may not be one encoded by the genetic code; (ii) one in which one or more of the amino acid residues in the targeting and/or MAp44 polypeptide or fragment thereof includes a substituent group, (iii) one in which the construct is fused with another compound, such as a compound to increase the half-life of the construct (for example, polyethylene glycol), (iv) one in which additional amino acids are fused to the construct (such as the targeting moiety or the MAp44 polypeptide or fragment thereof), such as a leader or secretory sequence or a sequence which is employed for purification of the construct, or (v) one in which the construct is fused with a larger polypeptide, i.e., human albumin, an antibody or Fc, for increased duration of effect. Such variants are deemed to be within the scope of those skilled in the art from the teachings herein.

In some embodiments, the variant of the construct contains conservative amino acid substitutions (defined further below) made at one or more predicted, preferably nonessential amino acid residues. A “nonessential” amino acid residue is a residue that is altered from the wild-type sequence of a protein without altering the biological activity, whereas an “essential” amino acid residue is required for biological activity. A “conservative amino acid substitution” is one in which the amino acid residue is replaced with an amino acid residue having a similar side chain.

Twenty amino acids are commonly found in proteins. Those amino acids can be grouped into nine classes or groups based on the chemical properties of their side chains. Substitution of one amino acid residue for another within the same class or group is referred to herein as a “conservative” substitution. Conservative amino acid substitutions can frequently be made in a protein without significantly altering the conformation or function of the protein. Substitution of one amino acid residue for another from a different class or group is referred to herein as a “non-conservative” substitution. In contrast, non-conservative amino acid substitutions tend to disrupt conformation and function of a protein. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). (See Table 1 below.)

TABLE 1
Example of amino acid classification

| | |
|---|---|
| Small/Aliphatic residues: | Gly, Ala, Val, Leu, Ile |
| Cyclic Amino Acid: | Pro |
| Hydroxyl-containing Residues: | Ser, Thr |
| Acidic Residues: | Asp, Glu |
| Amide Residues: | Asn, Gln |
| Basic Residues: | Lys, Arg |
| Imidazole Residue: | His |
| Aromatic Residues: | Phe, Tyr, Trp |
| Sulfur-containing Residues: | Met, Cys |

In some embodiments, the conservative amino acid substitution comprises substituting any of glycine (G), alanine (A), isoleucine (I), valine (V), and leucine (L) for any other of these aliphatic amino acids; serine (S) for threonine (T) and vice versa; aspartic acid (D) for glutamic acid (E) and vice versa; glutamine (Q) for asparagine (N) and vice versa; lysine (K) for arginine (R) and vice versa; phenylalanine (F), tyrosine (Y) and tryptophan (W) for any other of these aromatic amino acids; and methionine (M) for cysteine (C) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pKs of these two amino acid residues are not significant. Still other changes can be considered “conservative” in particular environments (see, e.g., BIOCHEMISTRY at pp. 13-15, 2^{nd} ed. Lubert Stryer ed. (Stanford University); Henikoff et al., Proc. Nat'l Acad. Sci. USA (1992) 89:10915-10919; Lei et al., J. Biol. Chem. (1995) 270(20):11882-11886).

Amino acid substitutions in the targeting moiety and/or the MAp44 polypeptide or fragment thereof of the construct are introduced to improve the functionality of the construct. For example, amino acid substitutions can be introduced into the targeting moiety of a targeting construct to increase binding affinity of the targeting moiety to its ligand(s), increase binding specificity of the targeting construct to its ligand(s), improve targeting of the targeting construct to desired sites, increase dimerization or multimerization of the targeting construct, and improve pharmacokinetics of the targeting construct. Similarly, amino acid substitutions can be introduced into the MAp44 polypeptide or fragment thereof of the construct to increase the functionality of the construct molecule and improve pharmacokinetics of the construct.

In some embodiments, the construct is fused with another compound, such as a compound to increase the half-life of the construct and/or to reduce potential immunogenicity of the construct (for example, polyethylene glycol, “PEG”). The PEG can be used to impart water solubility, size, slow rate of kidney clearance, and reduced immunogenicity to the construct. See e.g., U.S. Pat. No. 6,214,966. In the case of PEGylations, the fusion of a construct described herein to PEG can be accomplished by any means known to one skilled in the art. For example, PEGylation can be accomplished by first introducing a cysteine mutation into the targeting moiety or the MAp44 polypeptide or fragment thereof, followed by site-specific derivatization with PEG-maleimide. The cysteine can be added to the C-terminus of the construct. See, e.g., Tsutsumi et al. (2000) Proc. Natl. Acad. Sci. USA 97(15):8548-8553. Another modification which can be made to the construct involves biotinylation. In certain instances, it may be useful to have the construct biotinylated so that it can readily react with streptavidin. Methods for biotinylation of proteins are well known in the art. Additionally, chondroitin sulfate can be linked with the construct.

In some embodiments, the construct is fused to another moiety which further increases the targeting efficiency of the construct. For example, a construct comprising a B4 antibody can be fused to, e.g., a C2 antibody or another antibody that has the capability to bind or otherwise attach to an endothelial cell of a blood vessel (referred to as “vascular endothelial targeting amino acid ligand”). Exemplary vascular endothelial targeting ligands include, but are not limited to, VEGF, FGF, integrin, fibronectin, I-CAM, PDGF, or an antibody to a molecule expressed on the surface of a vascular endothelial cell.

In some embodiments, the construct is conjugated (such as fused) to a ligand for intercellular adhesion molecules. For example, the construct molecule can be conjugated to one or more carbohydrate moieties that bind to an intercellular adhesion molecule. The carbohydrate moiety facilitates localization of the construct molecule to the site of injury. The carbohydrate moiety can be attached to the construct molecule by means of an extracellular event such as a chemical or enzymatic attachment, or can be the result of an intracellular processing event achieved by the expression of appropriate enzymes. In some embodiments, the carbohydrate moiety binds to a particular class of adhesion molecules such as integrins or selectins, including E-selectin, L-selectin or P-selectin. In some embodiments, the carbohydrate moiety comprises an N-linked carbohydrate, for example the complex type, including fucosylated and sialylated carbohydrates. In some embodiments, the carbohydrate moiety is related to the Lewis X antigen, for example the sialylated Lewis X antigen.

For treatment of eye diseases such as AMD, the construct can be conjugated (such as fused) to an antibody that recognizes an epitope of the drusen. Other targeting molecules such as small targeting peptides can also be used. Other modifications of the construct include, for example, glycosylation, acetylation, phosphorylation, amidation, derivatization by known protecting/blocking groups, and the like.

The construct may include the addition of an immunologically active domain, such as an antibody epitope or other tag, to facilitate targeting or purification of the polypeptide. The use of 6×His and GST (glutathione S transferase) as tags is well known. Inclusion of a cleavage site at or near the fusion junction will facilitate removal of the extraneous polypeptide from the construct after purification. Other amino acid sequences that may be included in the construct include functional domains, such as active sites from enzymes such as a hydrolase, glycosylation domains, and cellular targeting signals.

Variants of the construct include polypeptides having an amino acid sequence sufficiently similar to the amino acid sequence of a construct described herein. The term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that contain a common structural domain that is at least about 45%, preferably about 75% through 98%, identical are defined herein as sufficiently similar. Variants include variants of constructs encoded by a polynucleotide that hybridizes to a polynucleotide of this invention or a complement thereof under stringent conditions. Such variants generally retain the functional activity of the constructs of this invention. Libraries of fragments of the polynucleotides can be used to generate a variegated population of fragments for screening and subsequent selection. For example, a library of fragments can be generated by treating a double-stranded PCR fragment of a polynucleotide with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with Si nuclease, and ligating the resulting fragment library into an expression vector. By this method, one can derive an expression library that encodes N-terminal and internal fragments of various sizes of the constructs of this invention.

Variants include constructs that differ in amino acid sequence due to mutagenesis. In addition, bioequivalent analogs of the constructs may also be constructed by making various substitutions on residues or sequences in the targeting moiety and/or the MAp44 polypeptide or fragment thereof.

In some embodiments, the construct is fused at its N-terminus to a signal peptide. Such signal peptides are useful for the secretion of the construct. Suitable signal peptides include, for example, the signal peptide of the CD5 protein (such as signal peptide of the human CD5 protein MPMGSLQPLATLYLLGMLVAS, SEQ ID NO: 54). In some embodiments, the signal peptide of the CR2 protein is used. For example, in some embodiments, the signal peptide of the human CR2 protein (MGAAGLLGVFLALVAPG, SEQ ID NO: 55 or MGAAGLLGVFLALVAPGVLG, SEQ ID NO: 56) is used.

### Construct Production Methods

The construct described herein can be produced using a variety of techniques known in the art of molecular biology and protein chemistry. For example, a nucleic acid encoding a construct described herein can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system such that it can be maintained in two different organisms, for example in mammalian or insect cells for expression and in a prokaryotic host for cloning and amplification.

Several possible vector systems are available for the expression of constructs from nucleic acids in mammalian cells. One class of vectors relies upon the integration of the desired gene sequences into the host cell genome. Cells which have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed, or introduced into the same cell by co-transfection (Wigler et al. (1979) Cell 16:77). A second class of vectors utilizes DNA elements which confer autonomously replicating capabilities to an extrachromosomal plasmid. These vectors can be derived from animal viruses, such as bovine papillomavirus (Sarver et al. (1982) Proc Natl Acad Sci USA, 79:7147), polyoma virus (Deans et al. (1984) Proc Natl Acad Sci USA 81: 1292), or SV 40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, lipofectin, electroporation, viral infection, dextran-mediated transfection, polybrene-mediated transfection, protoplast fusion, and direct microinjection.

Appropriate host cells for the expression of the construct include yeast, bacteria, insect, plant, and, as described above, mammalian cells. Of interest are bacteria such as E. coli, fungi such as Saccharomyces cerevisiae and Pichia pastoris, insect cells such as SF9, mammalian cell lines (e.g., human cell lines), as well as primary cell lines (e.g., primary mammalian cells). In some embodiments, the constructs can be expressed in Chinese hamster ovary (CHO) cells or in a suitable myeloma cell line such as (NSO). Suitable cell lines also include, for example, BHK-21 (baby hamster kidney) cells; 293 (human embryonic kidney) cells; HMEpC (Human Mammary Epithelial cells; 3T3 (mouse embryonic fibroblast) cells.

The targeting moiety and the one or more MAp44 polypeptides or fragments thereof may optionally be directly joined to each other, or may optionally be joined via a linker. Where the targeting moiety and MAp44 polypeptides or fragments thereof are directly joined, a hybrid vector is made where the DNA encoding the targeting and MAp44 polypeptides or fragments thereof are themselves directly ligated to each other using known scientific methods. Where a linker is used, a hybrid vector is made where the DNA encoding the targeting moiety is ligated to DNA encoding one end of the linker; and the DNA encoding the MAp44 polypeptide or fragment thereof is ligated to the other end of the linker. Methods are known for performing such ligations in proper orientation. Such ligation may be performed either in series, or as a three way ligation. Examples of sequences which may serve as the linker sequence in the present invention include short peptides of about 2 to about 16 amino acids in length. Among the peptide sequences useful as linkers in the present invention are (Gly-Ser)n, where n=1 to 8; (GlyGlyGlySer)n, where n=1 to 4; (GlySerSerGly)n, where n=1 to 4. Other examples of sequences useful as the linker sequence in the present invention include one or more short conserved region (SCR) domains from one or more of the following complement-related proteins: Factor H; complement receptor 1; complement receptor 2; Factor B; DAF; and others.

In some embodiments, a construct described herein can be expressed in, and purified from, transgenic animals (e.g., transgenic mammals). For example, a construct described herein can be produced in transgenic non-human mammals (e.g., rodents, sheep or goats) and isolated from milk as described in, e.g., Houdebine (2002) Curr Opin Biotechnol 13(6):625-629; van Kuik-Romeijn et al. (2000) Transgenic Res 9(2): 155-159; and Pollock et al. (1999) J Immunol Methods 231(1-2):147-157. Additional methods for producing proteins in mammalian milk products are described in, e.g., U.S. patent application publication nos. 200600105347 and 20040006776 and U.S. Pat. No. 7,045,676.

The constructs described herein can be produced from cells by culturing a host cell transformed with the expression vector containing nucleic acid encoding the construct, under conditions, and for an amount of time, sufficient to allow expression of the construct. Such conditions for protein expression will vary with the choice of the expression vector and the host cell, and will be easily ascertained by one skilled in the art through routine experimentation. For example, polypeptides expressed in E. coli can be refolded from inclusion bodies (see, e.g., Hou et al. (1998) Cytokine 10:319-30). Bacterial expression systems and methods for their use are well known in the art (see Current Protocols in Molecular Biology, Wiley & Sons, and Molecular Cloning—A Laboratory Manual—3rd Ed., Cold Spring Harbor Laboratory Press, New York (2001)). The choice of codons, suitable expression vectors and suitable host cells will vary depending on a number of factors, and may be easily optimized as needed. A construct described herein can be expressed in mammalian cells or in other expression systems including but not limited to yeast, baculovirus, and in vitro expression systems (see, e.g., Kaszubska et al. (2000) Protein Expression and Purification 18:213-220).

Following expression, the construct can be isolated. The term “purified” or “isolated” as applied to any of the proteins described herein (e.g., a construct, a targeting moiety, and/or a MAp44 polypeptide or fragment thereof) refers to a polypeptide that has been separated or purified from components (e.g., proteins or other naturally-occurring biological or organic molecules) which naturally accompany it, e.g., other proteins, lipids, and nucleic acid in a prokaryote expressing the proteins. Typically, a polypeptide is purified when it constitutes at least 60 (e.g., at least 65, 70, 75, 80, 85, 90, 92, 95, 97, or 99) %, by weight, of the total protein in a sample.

A construct described herein can be isolated or purified in a variety of ways known to those skilled in the art depending on what other components are present in the sample. Standard purification methods include electrophoretic, molecular, immunological, and chromatographic techniques, including ion exchange, hydrophobic, affinity, and reverse-phase HPLC chromatography. For example, a construct can be purified using a standard anti-construct antibody affinity column. Ultrafiltration and diafiltration techniques, in conjunction with protein concentration, are also useful. See, e.g., Scopes (1994) “Protein Purification, 3rd edition,” Springer-Verlag, New York City, New York. The degree of purification necessary will vary depending on the desired use. In some instances, no purification of the expressed polypeptide thereof will be necessary.

Methods for determining the yield or purity of a purified polypeptide are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

In some embodiments, a construct described herein can be synthesized de novo in whole or in part, using chemical methods well known in the art. For example, the component amino acid sequences can be synthesized by solid phase techniques, cleaved from the resin, and purified by preparative high performance liquid chromatography followed by chemical linkage to form a desired polypeptide. The composition of the synthetic peptides may be confirmed by amino acid analysis or sequencing.

Once expressed and/or purified, a construct described herein can be assayed for any one of a numbered of desired properties using in vitro or in vivo assays such as any of those described herein. For example, a construct described herein can be assayed for its ability to inhibit complement activity as described herein.

In some embodiments, endotoxin can be removed from the construct preparations. Methods for removing endotoxin from a protein sample are known in the art. For example, endotoxin can be removed from a protein sample using a variety of commercially available reagents including, without limitation, the ProteoSpin™ Endotoxin Removal Kits (Norgen Biotek Corporation), Detoxi-Gel Endotoxin Removal Gel (Thermo Scientific; Pierce Protein Research Products), MiraCLEAN® Endotoxin Removal Kit (Mirus), or Acrodisc™-Mustang® E membrane (Pall Corporation).

Methods for detecting and/or measuring the amount of endotoxin present in a sample (both before and after purification) are known in the art and commercial kits are available. For example, the concentration of endotoxin in a protein sample can be determined using the QCL-1000 Chromogenic kit (BioWhittaker), the limulus amebocyte lysate (LAL)-based kits such as the Pyrotell®, Pyrotell®-T, Pyrochrome®, Chromo-LAL, and CSE kits available from the Associates of Cape Cod Incorporated.

Following expression and purification, the constructs described herein can be modified. The modifications can be covalent or non-covalent modifications. Such modifications can be introduced into the constructs by, e.g., reacting targeted amino acid residues in the targeting moiety and/or the MAp44 polypeptide or fragment thereof with an organic derivatizing agent that is capable of reacting with selected side chains or terminal residues. Suitable sites for modification can be chosen using any of a variety of criteria including, e.g., structural analysis or amino acid sequence analysis of the constructs described herein.

In some embodiments, a construct described herein can be conjugated to a heterologous moiety. In embodiments where the heterologous moiety is a polypeptide, a construct and a corresponding heterologous moiety described herein can be joined by way of fusion protein. The heterologous moiety can be, e.g., a heterologous polypeptide, a therapeutic agent (e.g., a toxin or a drug), or a detectable label such as, but not limited to, a radioactive label, an enzymatic label, a fluorescent label, or a luminescent label. Suitable heterologous polypeptides include, e.g., an antigenic tag (e.g., FLAG, polyhistidine, hemagglutinin (HA), glutathione-S-transferase (GST), or maltose-binding protein (MBP)) for use in purifying the constructs. Heterologous polypeptides also include polypeptides that are useful as diagnostic or detectable markers, for example, luciferase, green fluorescent protein (GFP), or chloramphenicol acetyl transferase (CAT). Where the heterologous moiety is a polypeptide, the moiety can be incorporated into a fusion protein described herein, resulting in a fusion protein.

### Conjugates

In some embodiments, the fusion molecules described herein are created by linkage of two independently produced polypeptide fragments, e.g., an antibody (e.g., a Fab fragment of a B4 or C2 antibody) and a complement modulator polypeptide (e.g., a MAp44 polypeptide or fragment thereof). In certain embodiments, the targeting moiety is conjugated to the MAp44 polypeptide or fragment thereof through a lysine, cysteine, glutamate, aspartate, or arginine amino acid. A targeting moiety can be conjugated to a MAp44 polypeptide or fragment thereof through, e.g., a reaction comprising a thiolated targeting moiety, and a maleoyl-activated amine of the MAp44 polypeptide or fragment thereof; an EDC/NHS-activated targeting moiety, and an amine of the MAp44 polypeptide or fragment thereof; or an EDC/NHS-activated carboxylic acid of the MAp44 polypeptide or fragment thereof and an amine of the targeting moiety. Two proteins (e.g., a construct described herein and a heterologous moiety or the two constituent parts of a targeting construct) can, in some embodiments, be chemically cross-linked using any of a number of known chemical cross linkers. Examples of such cross linkers are those which link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyloxycarbonyla-methyl-a (2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful crosslinkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., m-maleimidobenzoyl-N-hydroxysuccinimide ester), an amino group and a carboxyl group (e.g., 4-[pazidosalicylamido]butylamine), and an amino group and a guanidinium group that is present in the side chain of arginine (e.g., p-azidophenyl glyoxal monohydrate).

In some embodiments, a fusion protein described herein can contain a heterologous moiety which is chemically linked to the fusion protein. For example, in some embodiments, a drug described herein, a fluorescent label, a paramagnetic label, a radioactive label, etc., can be directly conjugated to the amino acid backbone of the construct and/or targeting moiety (e.g., for use of the labeled construct for in vivo imaging studies).

In some embodiments, the constructs can be modified, e.g., with a moiety that improves the stabilization and/or retention of the constructs in circulation, e.g., in blood, serum, or other tissues. For example, a construct described herein can be PEGylated as described in, e.g., Lee et al. (1999) Bioconjug Chem 10(6): 973-8; Kinstler et al. (2002) Advanced Drug Deliveries Reviews 54:477-485; and Roberts et al. (2002) Advanced Drug Delivery Reviews 54:459-476. The stabilization moiety can improve the stability, or retention, of the construct by at least 1.5 (e.g., at least 2, 5, 10, 15, 20, 25, 30, 40, or 50 or more) fold.

In some embodiments, the constructs described herein can be glycosylated. In some embodiments, a construct described herein can be subjected to enzymatic or chemical treatment, or produced from a cell, such that the construct, targeting moiety, and/or MAp44 polypeptide or fragment thereof has reduced or absent glycosylation. Methods for producing polypeptides with reduced glycosylation are known in the art and described in, e.g., U.S. Pat. No. 6,933,368; Wright et al. (1991) EMBO J 10(10):2717-2723; and Co et al. (1993) Mol Immunol 30:1361-1367.

### Pharmaceutical Compositions

Also provided herein are pharmaceutical compositions comprising a construct comprising a MAp44 polypeptide or fragment thereof optionally linked to a targeting moiety and a pharmaceutically acceptable carrier. The pharmaceutical compositions may be suitable for a variety of modes of administration described herein, including for example systemic or localized administration. The pharmaceutical compositions can be in the form of eye drops, injectable solutions, or in a form suitable for inhalation (either through the mouth or the nose) or oral administration. The pharmaceutical compositions described herein can be packaged in single unit dosages or in multidosage forms.

In some embodiments, the pharmaceutical compositions comprise a construct comprising a MAp44 polypeptide or fragment thereof optionally linked to a targeting moiety and a pharmaceutically acceptable carrier suitable for administration to human. In some embodiments, the pharmaceutical compositions comprise a construct comprising a MAp44 polypeptide or fragment thereof optionally linked to a targeting moiety and a pharmaceutically acceptable carrier suitable for intraocular injection. In some embodiments, the pharmaceutical compositions comprise a construct comprising a MAp44 polypeptide or fragment thereof optionally linked to a targeting moiety and a pharmaceutically acceptable carrier suitable for topical application to the eye. In some embodiments, the pharmaceutical compositions comprise a construct comprising a MAp44 polypeptide or fragment thereof optionally linked to a targeting moiety and a pharmaceutically acceptable carrier suitable for intravenous injection. In some embodiments, the pharmaceutical compositions comprise a construct comprising a MAp44 polypeptide or fragment thereof optionally linked to a targeting moiety and a pharmaceutically acceptable carrier suitable for injection into the arteries (such as renal arteries).

The compositions are generally formulated as sterile, substantially isotonic, and in full compliance with all Good Manufacturing Practice (GMP) regulations of the U.S. Food and Drug Administration. In some embodiments, the composition is free of pathogen. For injection, the pharmaceutical composition can be in the form of liquid solutions, for example in physiologically compatible buffers such as Hank's solution or Ringer's solution. In addition, the pharmaceutical composition can be in a solid form and redissolved or suspended immediately prior to use. Lyophilized compositions are also included.

For oral administration, the pharmaceutical compositions can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

The present invention in some embodiments provides compositions comprising a construct comprising a MAp44 polypeptide or fragment thereof optionally linked to a targeting moiety and a pharmaceutically acceptable carrier suitable for administration to the eye. Such pharmaceutical carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Suitable pharmaceutical excipients include starch, glucose, lactose, sucrose, gelatin, malt, rice, sodium state, glycerol monostearate, glycerol, propylene, water, and the like. The pharmaceutical composition, if desired, can also contain minor amounts of wetting or emulsifying agents, or pH buffering agents. The construct and other components of the composition may be encased in polymers or fibrin glues to provide controlled release of the construct. These compositions can take the form of solutions, suspensions, emulsions, ointment, gel, or other solid or semisolid compositions, and the like. The compositions typically have a pH in the range of 4.5 to 8.0. The compositions must also be formulated to have osmotic values that are compatible with the aqueous humor of the eye and ophthalmic tissues. Such osmotic values will generally be in the range of from about 200 to about 400 milliosmoles per kilogram of water (“mOsm/kg”), but will preferably be about 300 mOsm/kg.

In some embodiment, the composition is formulated in accordance with routine procedures as a pharmaceutical composition adapted for injection intravenously, intraperitoneally, or intravitreally. Typically, compositions for injection are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions may further comprise additional ingredients, for example preservatives, buffers, tonicity agents, antioxidants and stabilizers, nonionic wetting or clarifying agents, viscosity-increasing agents, and the like.

Suitable preservatives for use in a solution include polyquaternium-1, benzalkonium chloride, thimerosal, chlorobutanol, methyl paraben, propyl paraben, phenylethyl alcohol, edetate disodium, sorbic acid, benzethonium chloride, and the like. Typically (but not necessarily) such preservatives are employed at a level of from 0.001% to 1.0% by weight.

Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5.

Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%.

Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like.

The use of viscosity enhancing agents to provide topical compositions with viscosities greater than the viscosity of simple aqueous solutions may be desirable to increase ocular absorption of the active compounds by the target tissues or increase the retention time in the eye. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents know to those skilled in the art. Such agents are typically employed at a level of from 0.01% to 2% by weight.

In some embodiments, there is provided a pharmaceutical composition for delivery of a nucleotide encoding a construct comprising a MAp44 polypeptide or fragment thereof optionally linked to a targeting moiety. The pharmaceutical composition for gene therapy can be in an acceptable diluent, or can comprise a slow release matrix in which the gene delivery vehicle or compound is imbedded. Alternatively, where the complete gene delivery system can be produced intact from recombinant cells, e.g., retroviral vectors, the pharmaceutical composition can comprise one or more cells which produce the gene delivery system.

In clinical settings, a gene delivery system for a gene therapeutic can be introduced into a subject by any of a number of methods. For instance, a pharmaceutical composition of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter, See U.S. Pat. No. 5,328,470, or by stereotactic injection, Chen et al. (1994), Proc. Natl. Acad. Sci., USA 91: 3054-3057. A polynucleotide encoding a construct can be delivered in a gene therapy construct by electroporation using techniques described, Dev et al. (1994), Cancer Treat. Rev. 20:105-115.

In some embodiments, there is provided a pharmaceutical composition for gene delivery to the eye. Ophthalmic solutions useful for storing and/or delivering expression vectors have been disclosed, for example, in WO03077796A2.

### Methods of Treating Diseases

The present application in some embodiments provides a method of inhibiting complement activation, inhibiting inflammation, or treating an inflammatory disease in an individual, comprising administering to the individual an effective amount of a composition comprising a construct, wherein the construct comprises a MAp44 polypeptide or fragment thereof. In some embodiments, the composition is administered by injection, such as parenteral, intravenous, subcutaneous, intraocular, intra-articular, or intramuscular injections. In some embodiments, there is provided a method of delivering a MAp44 polypeptide or fragment thereof to a site of tissue injury (such as non-ischemic tissue injury) in an individual, comprising administering to the individual an effective amount of a composition comprising a construct, wherein the construct comprises a MAp44 polypeptide or fragment thereof.

In some embodiments, the construct further comprises a targeting moiety (such as an antibody). In some embodiments, there is provided a method of inhibiting complement activation, inhibiting inflammation, or treating an inflammatory disease in an individual, comprising administering to the individual an effective amount of a composition comprising a construct, wherein the construct comprises (a) a MAp44 polypeptide or fragment thereof; and (b) an antibody or fragment thereof, wherein the antibody or fragment thereof specifically binds to Annexin IV or a phospholipid. In some embodiments, the composition is administered by injection, such as parenteral, intravenous, subcutaneous, intraocular, intra-articular, or intramuscular injections. In some embodiments, there is provided a method of delivering a MAp44 polypeptide or fragment thereof to a site of tissue injury (such as non-ischemic tissue injury) in an individual, comprising administering to the individual an effective amount of a composition comprising a construct, wherein the construct comprises (a) a MAp44 polypeptide or fragment thereof; and (b) an antibody or fragment thereof, wherein the antibody or fragment thereof specifically binds to Annexin IV or a phospholipid.

In some embodiments, there is provided a method of inhibiting complement activation (or inhibiting inflammation, for example complement-mediated inflammation) in a tissue in an individual, comprising administering to the individual an effective amount of a composition comprising a construct, wherein the construct comprises a MAp44 polypeptide or fragment thereof. In some embodiments, the tissue is any one of liver or portal tract, heart, muscle, brain, central or peripheral nervous system, gastrointestinal tract, lung, limb, arterial or venous vascular system, skin, bone marrow cells including red blood cells, platelets and nucleated cells, pancreas, eye, joint, and kidney. In some embodiments, the tissue is any one of eye, joint, and kidney. In some embodiments, the inflammation (such as complement mediated inflammation) is associated with tissue damage resulting from inflammatory disorders, transplant rejection (cellular or antibody mediated), pregnancy-related diseases, adverse drug reactions, autoimmune or immune complex disorders. In some embodiments, at least about 10% (including for example at least about any of 20%, 30%, 40%, 50%, 60%, 70%, 80%, 90%, 95%, or 100%) complement activation or inflammation is inhibited.

In some embodiments, there is provided a method of inhibiting complement activation (or inhibiting inflammation, for example complement-mediated inflammation) in a tissue in an individual, comprising administering to the individual an effective amount of a composition comprising a construct, wherein the construct comprises (a) an antibody or fragment thereof, wherein the antibody or fragment thereof specifically binds to Annexin IV; and (b) a MAp44 polypeptide or fragment thereof. In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or at risk of undergoing) tissue injury (such as non-ischemic injury) and/or oxidative damage. In some embodiments, the Annexin IV is produced by a nucleated cell (such as a mammalian cell). In some embodiments, the Annexin IV is a recombinant protein. In some embodiments, the construct is a fusion protein. In some embodiments, the targeting moiety and the MAp44 polypeptide or fragment thereof are linked via a linker (such as a peptide linker). In some embodiments, the tissue is any one of liver or portal tract, heart, muscle, brain, central or peripheral nervous system, gastrointestinal tract, lung, limb, arterial or venous vascular system, skin, bone marrow cells including red blood cells, platelets and nucleated cells, pancreas, eye, joint, and kidney. In some embodiments, the tissue is any one of eye, joint, and kidney. In some embodiments, the inflammation (such as complement mediated inflammation) is associated with tissue damage resulting from inflammatory disorders, transplant rejection (cellular or antibody mediated), pregnancy-related diseases, adverse drug reactions, autoimmune or immune complex disorders. In some embodiments, at least about 10% (including for example at least about any of 20%, 30%, 40%, 50%, 60%, 70%, 80%, 90%, 95%, or 100%) complement activation or inflammation is inhibited.

In some embodiments, there is provided a method of inhibiting complement activation (or inhibiting inflammation for example complement-mediated inflammation) in a tissue in an individual, comprising administering to the individual an effective amount of a composition comprising a construct, wherein the construct comprises (a) an antibody or fragment thereof, wherein the antibody or fragment thereof specifically binds to a phospholipid; and (b) a MAp44 polypeptide or fragment thereof. In some embodiments, the antibody or fragment thereof competitively inhibits the binding of a pathogenic antibody (such as monoclonal antibody C2) to the phospholipid. In some embodiments, the antibody or antibody fragment thereof binds to the same epitope as a pathogenic antibody (such as monoclonal antibody C2) to the phospholipid. In some embodiments, the phospholipid is present on the surface of a cell (or in a pathological structure (e.g., drusen)) in an individual that is in or adjacent to a tissue undergoing (or at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the antibody or fragment thereof binds to malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the MAp44 polypeptide or fragment thereof are linked via a linker (such as a peptide linker). In some embodiments, the tissue is any one of liver or portal tract, heart, muscle, brain, central or peripheral nervous system, gastrointestinal tract, lung, limb, arterial or venous vascular system, skin, bone marrow cells including red blood cells, platelets and nucleated cells, pancreas, eye, joint, and kidney. In some embodiments, the tissue is any one of eye, joint, and kidney. In some embodiments, the inflammation (such as complement mediated inflammation) is associated with tissue damage resulting from inflammatory disorders, transplant rejection (cellular or antibody mediated), pregnancy-related diseases, adverse drug reactions, autoimmune or immune complex disorders. In some embodiments, at least about 10% (including for example at least about any of 20%, 30%, 40%, 50%, 60%, 70%, 80%, 90%, 95%, or 100%) complement activation or inflammation is inhibited.

In some embodiments, there is provided a method of inhibiting complement activation (or inhibiting inflammation, for example complement-mediated inflammation) in a tissue having an oxidative damage in an individual, comprising administering to the individual an effective amount of a composition comprising a construct, wherein the construct comprises a MAp44 polypeptide or fragment thereof. In some embodiments, the tissue is any one of liver or portal tract, heart, muscle, brain, central or peripheral nervous system, gastrointestinal tract, lung, limb, arterial or venous vascular system, skin, bone marrow cells including red blood cells, platelets and nucleated cells, pancreas, eye, joint, and kidney. In some embodiments, the tissue is any one of eye, joint, and kidney. In some embodiments, the inflammation (such as complement mediated inflammation) is associated with tissue damage resulting from inflammatory disorders, transplant rejection (cellular or antibody mediated), pregnancy-related diseases, adverse drug reactions, autoimmune or immune complex disorders. In some embodiments, at least about 10% (including for example at least about any of 20%, 30%, 40%, 50%, 60%, 70%, 80%, 90%, 95%, or 100%) complement activation or inflammation is inhibited.

In some embodiments, there is provided a method of inhibiting complement activation (or inhibiting inflammation, for example complement-mediated inflammation) in a tissue having an oxidative damage in an individual, comprising administering to the individual an effective amount of a composition comprising a construct, wherein the construct comprises (a) an antibody or fragment thereof, wherein the antibody or fragment thereof specifically binds to Annexin IV; and (b) a MAp44 polypeptide or fragment thereof. In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or at risk of undergoing) tissue injury (such as non-ischemic injury) and/or oxidative damage. In some embodiments, the Annexin IV is produced by a nucleated cell (such as a mammalian cell). In some embodiments, the Annexin IV is a recombinant protein. In some embodiments, the construct is a fusion protein. In some embodiments, the targeting moiety and the MAp44 polypeptide or fragment thereof are linked via a linker (such as a peptide linker). In some embodiments, the tissue is any one of liver or portal tract, heart, muscle, brain, central or peripheral nervous system, gastrointestinal tract, lung, limb, arterial or venous vascular system, skin, bone marrow cells including red blood cells, platelets and nucleated cells, pancreas, eye, joint, and kidney. In some embodiments, the tissue is any one of eye, joint, and kidney. In some embodiments, the inflammation (such as complement mediated inflammation) is associated with tissue damage resulting from inflammatory disorders, transplant rejection (cellular or antibody mediated), pregnancy-related diseases, adverse drug reactions, autoimmune or immune complex disorders. In some embodiments, at least about 10% (including for example at least about any of 20%, 30%, 40%, 50%, 60%, 70%, 80%, 90%, 95%, or 100%) complement activation or inflammation is inhibited.

In some embodiments, there is provided a method of inhibiting complement activation (or inhibiting inflammation, for example complement-mediated inflammation) in a tissue having an oxidative damage in an individual, comprising administering to the individual an effective amount of a composition comprising a construct, wherein the construct comprises (a) an antibody or fragment thereof, wherein the antibody or fragment thereof specifically binds to a phospholipid; and (b) a MAp44 polypeptide or fragment thereof. In some embodiments, the antibody or fragment thereof competitively inhibits the binding of a pathogenic antibody (such as monoclonal antibody C2) to the phospholipid. In some embodiments, the antibody or antibody fragment thereof binds to the same epitope as a pathogenic antibody (such as monoclonal antibody C2) to the phospholipid. In some embodiments, the phospholipid is present on the surface of a cell (or in a pathological structure (e.g., drusen)) in an individual that is in or adjacent to a tissue undergoing (or at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the antibody or fragment thereof binds to malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the MAp44 polypeptide or fragment thereof are linked via a linker (such as a peptide linker). In some embodiments, the tissue is any one of liver or portal tract, heart, muscle, brain, central or peripheral nervous system, gastrointestinal tract, lung, limb, arterial or venous vascular system, skin, bone marrow cells including red blood cells, platelets and nucleated cells, pancreas, eye, joint, and kidney. In some embodiments, the tissue is any one of eye, joint, and kidney. In some embodiments, the inflammation (such as complement mediated inflammation) is associated with tissue damage resulting from inflammatory disorders, transplant rejection (cellular or antibody mediated), pregnancy-related diseases, adverse drug reactions, autoimmune or immune complex disorders. In some embodiments, at least about 10% (including for example at least about any of 20%, 30%, 40%, 50%, 60%, 70%, 80%, 90%, 95%, or 100%) complement activation or inflammation is inhibited.

In some embodiments, there is provided a method of treating an inflammatory disease (or a disease involving oxidative damage) in an individual, comprising administering to the individual an effective amount of a composition comprising a construct, wherein the construct comprises a MAp44 polypeptide or fragment thereof. In some embodiments, the inflammatory disease is any of inflammatory disorders, transplant rejection (cellular or antibody mediated, such as hyperacute xenograft injection), pregnancy-related diseases, adverse drug reactions (such as drug allergy and IL-2 induced vascular leakage syndrome), autoimmune or immune complex disorders.

In some embodiments, there is provided a method of treating an inflammatory disease (or a disease involving oxidative damage) in an individual, comprising administering to the individual an effective amount of a composition comprising a construct, wherein the construct comprises (a) an antibody or fragment thereof, wherein the antibody or fragment thereof specifically binds to Annexin IV; and (b) a MAp44 polypeptide or fragment thereof. In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or at risk of undergoing) tissue injury (such as non-ischemic injury) and/or oxidative damage. In some embodiments, the Annexin IV is produced by a nucleated cell (such as a mammalian cell). In some embodiments, the Annexin IV is a recombinant protein. In some embodiments, the construct is a fusion protein. In some embodiments, the targeting moiety and the MAp44 polypeptide or fragment thereof are linked via a linker (such as a peptide linker). In some embodiments, the inflammatory disease is any of inflammatory disorders, transplant rejection (cellular or antibody mediated, such as hyperacute xenograft injection), pregnancy-related diseases, adverse drug reactions (such as drug allergy and IL-2 induced vascular leakage syndrome), autoimmune or immune complex disorders.

In some embodiments, there is provided a method of treating an inflammatory disease (or a disease involving oxidative damage) in an individual, comprising administering to the individual an effective amount of a composition comprising a construct, wherein the construct comprises (a) an antibody or fragment thereof, wherein the antibody or fragment thereof specifically binds to a phospholipid; and (b) a MAp44 polypeptide or fragment thereof. In some embodiments, the antibody or fragment thereof competitively inhibits the binding of a pathogenic antibody (such as monoclonal antibody C2) to the phospholipid. In some embodiments, the antibody or antibody fragment thereof binds to the same epitope as a pathogenic antibody (such as monoclonal antibody C2) to the phospholipid. In some embodiments, the phospholipid is present on the surface of a cell (or in a pathological structure (e.g., drusen)) in an individual that is in or adjacent to a tissue undergoing (or at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the antibody or fragment thereof binds to malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the MAp44 polypeptide or fragment thereof are linked via a linker (such as a peptide linker). In some embodiments, the inflammatory disease is any of inflammatory disorders, transplant rejection (cellular or antibody mediated, such as hyperacute xenograft injection), pregnancy-related diseases, adverse drug reactions (such as drug allergy and IL-2 induced vascular leakage syndrome), autoimmune or immune complex disorders.

Also provided are methods of delivering a construct comprising a MAp44 polypeptide or fragment thereof optionally linked to a targeting moiety to a site of tissue injury in an individual, comprising administering to the individual an effective amount of a composition comprising a construct, wherein the construct comprises (a) a MAp44 polypeptide or fragment thereof; and/or (b) an antibody or fragment thereof, wherein the antibody or fragment thereof specifically binds to Annexin IV or a phospholipid.

In some embodiments, there is provided a method of inhibiting complement activation, inhibiting inflammation, or treating an inflammatory disease in an individual, comprising administering to the individual a vehicle for introducing into the individual an exogenous nucleic acid comprising a sequence for expression of a construct comprising a MAp44 polypeptide or fragment thereof optionally linked to a targeting moiety, wherein the vehicle is a vector selected from the group consisting of an adenovirus, a retrovirus, an adeno-associated virus and a plasmid.

Also provided are methods of delivering a construct comprising a MAp44 polypeptide or fragment thereof optionally linked to a targeting moiety to a site of tissue injury in an individual, comprising administering to the individual a vehicle for introducing into the patient an exogenous nucleic acid comprising a sequence for expression of a construct described above, wherein the vehicle is a vector selected from the group consisting of an adenovirus, a retrovirus, an adeno-associated virus and a plasmid.

In some embodiments, the disease to be treated is an ocular disease. In some embodiments, the disease is an ocular disease associated with complement activation. In some embodiments, the disease is age-related macular degeneration (“AMD”), including wet AMD and dry AMD. Other ocular diseases that can be treated by methods described herein include, but are not limited to, CMV retinitis, macular edema, uveitis, glaucoma, diabetic retinopathy, retinitis pigmentosa, retinal detachment, proliferative vitreoretinopathy and ocular melanoma.

In some embodiments, the disease to be treated is inflammatory arthritis.

In some embodiments, the disease to be treated is a kidney disease, including but not limited to, acute kidney injury, glomerulonephritis, chronic kidney disease, and focal segmental glomerulosclerosis.

In some embodiments, the disease to be treated is an inflammatory disorder, which includes, but is not limited to, burns, endotoxemia, septic shock, adult respiratory distress syndrome, cardiopulmonary bypass, hemodialysis, anaphylactic shock, asthma, angioedema, Crohn's disease, sickle cell anemia, poststreptococcal glomerulonephritis, membranous nephritis, and pancreatitis.

In some embodiments, the disease to be treated is a pregnancy-related disease, which includes, but is not limited to, HELLP (Hemolytic anemia, elevated liver enzymes, and low platelet count), recurrent fetal loss, and pre-eclampsia.

In some embodiments, the disease to be treated is an autoimmune or immune complex disorder, which include, but is not limited to, myasthenia gravis, Alzheimer's disease, multiple sclerosis, neuromyelitis optica, rheumatoid arthritis, osteoarthritis, systemic lupus erythematosus, lupus nephritis, IgG4 associated diseases, insulin-dependent diabetes mellitus, acute disseminated encephalomyelitis, Addison's disease, antiphospholipid antibody syndrome, thrombotic thrombycytopenic purpura, autoimmune hepatitis, Crohn's disease, Goodpasture's syndromes, Graves' disease, Guillain-Barre syndrome, Hashimoto's disease, idiopathic thrombocytopenic purpura, pemphigus, Sjogren's syndrome, Takayasu's arteritis, autoimmune glomerulonephritis, membranoproliferative glomerulonephritis type II, membranous disease, paroxysmal nocturnal hemoglobinuria, age-related macular degeneration, diabetic maculopathy, uveitis, retinal degeneration disorders, diabetic nephropathy, focal segmental glomerulosclerosis, ANCA associated vasculitis, hemolytic uremic syndrome, Shiga-toxin-associated hemolytic uremic syndrome, and atypical hemolytic uremic syndrome. In some embodiments, the disease to be treated is an autoimmune glomerulonephritis, which includes, but is not limited to, immunoglobulin A nephropathy or membranoproliferative glomerularnephritis type I.

### Diseases to be Treated

The treatment methods described herein can be used for treating a variety of diseases, including, but not limited to, inflammatory diseases, transplant rejections, pregnancy-related diseases, adverse drug reactions, tissue damage resulting from ischemia-reperfusion injury, ocular diseases, kidney diseases, joint diseases, and autoimmune or immune complex disorders. In some embodiments, the disease to be treated includes, but is not limited to, systemic lupus erythematosus and glomerulonephritis, rheumatoid arthritis, cardiopulmonary bypass and hemodialysis, hyperacute rejection in organ transplantation, myocardial infarction, ischemia/reperfusion injury, antibody-mediated allograft rejection, for example, in the kidneys, and adult respiratory distress syndrome. Moreover, other inflammatory conditions and autoimmune/immune complex diseases are also closely associated with complement activation, including, but not limited to, thermal injury, severe asthma, anaphylactic shock, bowel inflammation, urticaria, angioedema, vasculitis, multiple sclerosis, myasthenia gravis, myocarditis, membranoproliferative glomerulonephritis, atypical hemolytic uremic syndrome, Sjogren's syndrome, renal and pulmonary ischemia/reperfusion, and other organ-specific inflammatory disorders. Accordingly, in some embodiments, the methods described herein are particularly useful for treating a complement-mediated disease including, but not limited to, inflammatory disease, a transplant rejection, pregnancy-related disease, adverse drug reaction, tissue damage resulting from ischemia-reperfusion injury, ocular disease, kidney disease, joint disease, or an autoimmune or immune complex disorder. In some embodiments, also provided herein are methods of treating a complement-mediated disease in an individual, comprising administering to the individual an effective amount of any of the compositions (such as a composition comprising a construct) described herein.

The methods described herein are particularly useful for treating inflammatory diseases including, but not limited to, burns, endotoxemia, septic shock, adult respiratory distress syndrome, cardiopulmonary bypass, hemodialysis, anaphylactic shock, asthma, angioedema, Crohn's disease, sickle cell anemia, poststreptococcal glomerulonephritis, membranous nephritis, pancreatitis, rheumatoid arthritis, inflammatory arthritis, inflammatory bowel disease, acute lung injury, and disseminated intravascular coagulation (DIC). In some embodiments, the inflammation (such as complement-mediated inflammation) is associated with tissue damage resulting from inflammatory or autoinflammatory disorders, transplant rejection (cellular- or antibody-mediated), pregnancy-related diseases, adverse drug reactions, degenerative, neovascular, hemolytic, thrombotic, vasculitic, arthritic, regenerative, traumatic, autoimmune or immune complex disorders.

The compositions described herein are also useful for treating a transplant rejection including, but not limited to, a hyperacute transplant rejection, antibody-mediated transplant rejection, cellular-mediated transplant rejection, acute transplant rejection, and chronic transplant rejection. In some embodiments, the transplant is a xenograft, an allograft, or an isograft. In some embodiments, the transplant is a fluid, a cell, a tissue or an organ. In some embodiments, the transplant is selected from the group consisting of: heart, liver, kidney, lung, pancreas, intestine, stomach, testis, hand, arm, leg, uterus, ovary, and thymus. In some embodiments, the transplant is selected from the group consisting of: bone, tendons, cornea, skin, heart valve, islets of Langerhans, bone marrow, hematopoietic stem cell, blood transfusion, and vein. In some embodiments, the transplant is a heart, liver or kidney. Transplant rejections can result in several complications such as graft-versus-host disease. In some embodiments, a complement-mediated disease is graft-versus-host disease.

The methods described herein are also particularly useful for treating a pregnancy-related disease including, but not limited to, HELLP (Hemolytic anemia, elevated liver enzymes, and low platelet count), recurrent fetal loss, atypical hemolytic uremic syndrome, fetal hypoxia syndrome, hypertensive disease, and pre-eclampsia.

In addition, the methods described herein are useful for treating an adverse drug reaction including, but not limited to, a drug allergy, a radiographic contrast media allergy, and IL-2 induced vascular leakage.

The methods described herein are also useful for treating tissue damage resulting from ischemia-reperfusion injury following, but not limited to, acute myocardial infarction, aneurysm, aneurysm repair, deep hypothermic circulatory arrest, tourniquet use, solid organ transplant, stroke including perinatal stroke, hemorrhagic shock, crush injury, multiple organ failure, hemodialysis, hypovolemic shock, spinal cord injury, traumatic brain injury, intestinal ischemia, retinal ischemia, cardiopulmonary bypass, emergency coronary surgery for failed percutaneous transluminal coronary angioplasty (PCTA), and any vascular surgery with blood vessel cross clamping, pancreatitis after manipulation of pancreatic or bile duct. In some embodiments, tissue damage can be treated before, during, or after the ischemic event (such as intestinal ischemia) that triggers ischemia-reperfusion injury.

In some embodiments, tissue damage is treated with any of the methods disclosed herein by administering a construct (or a composition comprising the construct or a vehicle for expression of the construct) disclosed herein before reperfusion. In some embodiments, tissue damage is treated with any of the methods disclosed herein by administering a construct (or a composition comprising the construct or a vehicle for expression of the construct) disclosed herein after reperfusion. In some embodiments, the ischemia-reperfusion injury is selected from the group consisting of: myocardial ischemia-reperfusion, renal ischemia-reperfusion injury, gastrointestinal ischemia-reperfusion injury, hepatic ischemia-reperfusion injury, skeletal muscle ischemia-reperfusion injury, cerebral ischemia-reperfusion injury, pulmonary ischemia-reperfusion injury, intestine ischemia-reperfusion injury, retinal ischemia-reperfusion injury, and joint ischemia-reperfusion injury. In some embodiments, tissue damage is caused by oxidative damage.

There are instances when a therapy or surgery induces a reperfusion but not an ischemia (referred herein as non-ischemia reperfusion injury). Such therapy or surgery includes, but is not limited to, pharmacological thrombolysis, including intravenous and endovascular therapies for stroke, acute coronary syndromes, peripheral arterial occlusion, pulmonary embolus, renal artery occlusion, mechanical thromnbolysis, e.g. percutaneous coronary intervention, peripheral arterial angioplasty, visceral arterial angioplasty, coronary artery bypass grafting, carotid endarterectorny, rnesenteric ischemia, shock including hemorrhagic, cardiogenic, neurogenic, anaphylactic, flap-failure, e.g. plastic surgery, re-implantation of digits and limbs, and strangulated bowel. Accordingly, in some embodiments, tissue damage resulting from non-ischemia reperfusion injury is treated with any of the methods disclosed herein by administering a construct (or a composition comprising the construct or a vehicle for expression of the construct) disclosed herein.

The methods described herein are also particularly useful for treating a kidney disease including, but not limited to, acute kidney injury, hemolytic uremic syndrome, glomerulonephritis, membranous glomerulonephritis, mesangioproliferative glomerulonephritis, acute postinfectious glomeruilonephritis (such as poststreptococcal glomerulonephritis), cryoglobulineric glorerulonephritis, lupus nephritis, membranoproliferative glomerulonephritis (such as mesangiocapillary glomerulonephritis), dense deposit disease, minimal change disease, diabetic nephropathy, Henoch-Schonlein purpura nephritis, IgA nephropathy, chronic kidney disease, delayed graft function of a kidney transplant, acute and chronic renal transplant rejection, proteinuric renal disease and nephrotic syndrome, hypertensive kidney disease, and focal segmental glomerulosclerosis. In some embodiments, the kidney disease is a glomerular disease. For example, the methods are useful for treating glomerular disease that leads to binding of natural IgM to damaged glomerulus. In some embodiments, damaged glomerulus can be a result of mechanical, metabolic, chemical, oxidative or immunologic stress. In some embodiments, damaged glomerulus can be a result of ischemia, diabetes, hypertension, and secondary focal segmental glomerulosclerosis. Symptoms of damaged glomerulus include an inflammatory response such as cytokine release and fibrosis such as collagen mesangial matrix deposition, tubular cell damage, and tubulointerstitial fibrosis. The methods are also useful for treating kidney disease such a glomerulonephritis which is inflammation of the glomerulus. Glomerulonephritis is commonly associated with deposition of electron dense material in the glomerulus which contains complement components, including C3. The methods are also useful for treating acute kidney injury associated with renal ischemia. Ischemia is the leading cause of acute kidney injury. Ischemia and subsequent reperfusion elicit acute kidney injury through endothelial dysfunction, leukocyte-mediated inflammation and decreased microvascular blood flow that can lead to rarefaction of the peritubular capillaries, shifting the fragile balance of oxygen supply and demand to the corticomedullary junction toward a negative oxygen balance. The shift in balance causes a hypoxic environment and can lead to accumulation of fibrosis and subsequent development of chronic kidney disease. In some embodiments, the kidney disease is due to a factor H deficiency.

The methods described herein are also useful for treating a joint disease including, but not limited to, arthritis (such as rheumatoid arthritis) and joint inflammation associated with infection (such as hepatitis B infection), inflammatory disease (such as inflammatory bowel disease) or autoimmune disease (such as systemic lupus erythematosus). In some embodiments, methods provided herein are useful for treating a joint disease including, but not limited to, arthritis, amyloid arthropathy, amyloidosis, ankylosing spondylitis, carpal tunnel syndrome, temporal arteritis, polymyalgia rheumatica, polyarthralgia, tendinitis, Whipple's disease, bursitis, trigeminal neuralgia, fibromyoma, fibrositis, autoimmune arthritis, rheumatoid arthritis, juvenile arthritis, psoriatic arthritis, lupus arthritis, polyarthritis, inflammatory arthritis not resulting from an autoimmune disease or disorder, such as an infectious arthritis, i.e., joint pain, soreness, stiffness and swelling caused by an infectious agent such as bacteria (including Mycoplasma), viruses, fungi, septic arthritis, or osteoarthritis. Joint disease can be associated with symptoms such as joint stiffness, pain, weakness, joint fatigue, tenderness and swelling. Accordingly, in some embodiments, symptoms of joint disease can be treated d with any of the methods disclosed herein by administering a construct (or a composition comprising the construct or a vehicle for expression of the construct) disclosed herein. For example, the compositions are useful for treating arthritis or symptoms of arthritis. In some embodiments, the arthritis is selected from the group consisting of: rheumatoid arthritis, juvenile onset rheumatoid arthritis, psoriatic arthritis, and lupus arthritis. In some embodiments the arthritis is osteoarthritis. In some embodiments, the arthritis is infectious arthritis caused by a bacterial pathogen, such as Haemophilus influenzae, Gonoccous spp., Mycoplasma spp. Meingococcus spp., Pneumococcus spp., Streptococcus spp., Staphyloccus spp., Salmonella spp., Brucella spp., Neisseria spp., Streptobacillus moniliformis (Haverhill fever), Mycobacterium tuberculosis, Treponema pallidum (syphilis), Treponema pertenue (yaws), or Rickettsia spp. In some embodiments, the arthritis is infectious arthritis caused by a viral pathogen, such as a rubella virus, a mumps virus, a varicella-zoster virus, an adenovirus, an echovirus, a herpes simplex virus, a cytomegalovirus, a parvovirus, a retrovirus, and alphavirus, or a hepatitis virus. In some embodiments, the arthritis is infectious arthritis caused by a fungus, such as Coccidioides spp., Histoplasmoa spp., Blastomyces spp., Cryptococcus spp., Candida spp., or Sporothrix spp. As another example, the compositions are useful for treating a joint disease or symptoms of a joint disease. In some embodiments, the joint disease is arthritis, amyloid arthropathy, amyloidosis, ankylosing spondylitis, carpal tunnel syndrome, temporal arteritis, polymyalgia rheumatica, polyarthralgia, tendinitis, Whipple's disease, bursitis, trigeminal neuralgia, fibromyoma, and fibrositis. In some embodiments, the joint disease is associated with arthritis. In some embodiments, the joint disease precedes the development of arthritis. In some embodiments, the joint disease develops due to the onset of arthritis.

Rheumatoid arthritis affects approximately 1% of the population, with women affected three times more commonly than men. Rheumatoid arthritis and juvenile onset rheumatoid arthritis are systemic diseases with numerous pathologic manifestations in addition to their joint inflammatory aspects. In rheumatoid arthritis, these manifestations include vasculitis (inflammation of the blood vessels), which can affect nearly any organ system and can cause numerous pathologic sequelae including polyneuropathy, cutaneous ulceration, and visceral infarction. Pleuropulmonary manifestations include pleuritis, interstitial fibrosis, pleuropulmonary nodules, pneumonitis, and arteritis. Other manifestations include the development of inflammatory rheumatoid nodules on a variety of periarticular structures such as extensor surfaces, as well as on pleura and meninges. Weakness and atrophy of skeletal muscle are common. Many patients with systemic lupus erythematosis also develop joint inflammation referred to as lupus arthritis. Systemic lupus erythematosis is an autoimmune disease of unknown cause in which numerous different cells, tissues, and organs are damaged by pathogenic autoantibodies and immune complexes. Clinical manifestations of systemic lupus erythematosis are numerous and include a variety of maculopapular rashes, nephritis, cerebritis, vasculitis, hematologic abnormalities including cytopenias and coagulopathies, pericarditis, myocarditis, pleurisy, gastrointestinal symptoms, and the aforementioned joint inflammation. Osteoarthritis represents the most common chronic joint disease. It is manifested by pain, stiffness, and swelling of the involved joints. Articular cartilage, responsible for the most critical mechanical functions of the joint, is the major target tissue of osteoarthritis and the breakdown of articular cartilage in osteoarthritis is mediated by various enzymes such as metalloproteinases, plasmin, and cathepsin, which are in turn stimulated by various factors that can also act as inflammatory mediators. These factors include cytokines such as interleukin-1, which is known to activate the pathogenic cartilage and synovial proteases. Synovial inflammation becomes more frequent as the disease progresses. Psoriatic arthritis is a chronic inflammatory joint disorder that affects 5 to 8% of people with psoriasis. A significant percentage of these individuals (one-fourth) develop progressive destructive disease. Twenty five percent of psoriasis patients with joint inflammation develop symmetric joint inflammation resembling the joint inflammation manifestations of rheumatoid arthritis, and over half of these go on to develop varying degrees of joint destruction.

The methods described herein are useful for treating an autoimmune or immune complex including, but not limited to, but is not limited to, myasthenia gravis, Alzheimer's disease, multiple sclerosis, emphysema, obesity, neuromyelitis optica, rheumatoid arthritis, osteoarthritis, systemic lupus erythematosus, lupus nephritis, IgG4 associated diseases, insulin-dependent diabetes mellitus, acute disseminated encephalomyelitis, Addison's disease, antiphospholipid antibody syndrome, thrombotic thrombycytopenic purpura, autoimmune hepatitis, Crohn's disease, Goodpasture's syndromes, Graves' disease, Guillain-Barre syndrome, Hashimoto's disease, idiopathic thrombocytopenic purpura, pemphigus, Sjogren's syndrome, Takayasu's arteritis, autoimmune glomerulonephritis, dense deposit disease (also known as membranoproliferative glomerulonephritis type II), membranous disease, paroxysmal nocturnal hemoglobinuria, age-related macular degeneration, diabetic maculopathy, uveitis, retinal degeneration disorders, diabetic nephropathy, focal segmental glomerulosclerosis, ANCA associated vasculitis, hemolytic uremic syndrome, Shiga-toxin-associated hemolytic uremic syndrome, atypical hemolytic uremic syndrome, and inflammation associated cardiopulmonary bypass and hemodialysis. In some embodiments, the disease to be treated d is an autoimmune glomerulonephritis, which includes, but is not limited to, immunoglobulin A nephropathy or membranoproliferative glomerularnephritis type I. In some embodiments, an autoimmune or immune complex disorder is an inflammatory disease.

The methods described herein are particularly useful for treating ocular diseases including, but not limited to, age-related macular degeneration (“AMD”), including wet AMD and dry AMD, CMV retinitis, macular edema, uveitis, glaucoma, diabetic retinopathy, retinitis pigmentosa, retinal detachment, proliferative vitreoretinopathy and ocular melanoma. For example, the methods are useful for treating age-related macular degeneration (AMD). AMD is clinically characterized by progressive loss of central vision which occurs as a result of damage to the photoreceptor cells in an area of the retina called the macula. AMD has been broadly classified into two clinical states: a wet form and a dry form, with the dry form making up to 80-90% of total cases. The dry form is characterized clinically by the presence of macular drusen, which are localized deposits between the retinal pigment epithelium (RPE) and the Bruch's membrane, and by geographic atrophy characterized by RPE cell death with overlying photoreceptor atrophy. Wet AMD, which accounts for approximately 90% of serious vision loss, is associated with neovascularization in the area of the macular and leakage of these new vessels. The accumulation of blood and fluid can cause retinal detachment followed by rapid photoreceptor degeneration and loss of vision. It is generally accepted that the wet form of AMD is preceded by and arises from the dry form.

Analysis of the contents of drusen in AMD patients has shown a large number of inflammatory proteins including amyloid proteins, coagulation factors, and a large number of proteins of the complement pathway. A genetic variation in the complement factor H substantially raises the risk of age-related macular degeneration (AMD), suggesting that uncontrolled complement activation underlies the pathogenesis of AMD. Edward et al., Science 2005, 308:421; Haines et al., Science 2005, 308:419; Klein et al., Science 308:385-389; Hageman et al., Proc. Natl. Acad. Sci. USA 2005, 102:7227.

In some embodiments, the methods described herein can be used to treat cytomegalovirus (CMV) retinitis. CMV retinitis is an infection that causes inflammation of the photoreceptor cells in the retina. CMV is typically rare in immunocompetent individuals. However, individuals who are immunocompromised, e.g., by diseases, transplants, or chemotherapy, are particularly susceptible to CMV retinitis. Retinitis usually begins in one eye, but often progresses to the other eye. Without treatment, progressive damage to the retina can lead to blindness in 4-6 months or less.

In some embodiments, the methods described herein can be used to treat macular edema. Macular edema occurs when fluid and protein deposits collect on or under the macula of the eye, causing it to thicken and swell. The swelling may distort an individual's central vision, as the macula holds tightly packed cones that provide sharp, clear central vision to enable a person to see detail, form, and color that is directly in the direction of gaze. Macular edema can be classified into two types. Cystoid macular edema (CME) involves fluid accumulation in the outer plexiform layer secondary to abnormal perifoveal retinal capillary permeability. Diabetic macular edema (DME) is similarly caused by leaking macular capillaries. DME is the most common cause of visual loss in both proliferative and non-proliferative diabetic retinopathy.

In certain embodiments, the methods described herein can be used to treat uveitis, i.e., inflammation of the uvea (the iris, ciliary body, and choroid of the eye beneath the sclera). Uveitis is typically associated with eye infections, eye injuries, and/or autoimmune disorders. However, in many cases, the cause is unknown. The most common form of uveitis is anterior uveitis, which involves inflammation in iris. Posterior uveitis affects the choroid, a layer of blood vessels and connective tissue in the middle part of the eye. Another form of uveitis is pars planitis. This inflammation affects the narrowed area (pars plana) between the iris and the choroid.

In certain embodiments, the methods described herein can be used to treat glaucoma, a group of eye conditions that lead to damage to the optic nerve, and loss of vision. The nerve damage involves loss of retinal ganglion cells in a characteristic pattern. The many different subtypes of glaucoma can all be considered to be a type of optic neuropathy. Raised intraocular pressure (above 21 mmHg or 2.8 kPa) is the most important and only modifiable risk factor for glaucoma. Intraocular pressure is a function of production of liquid aqueous humor by the ciliary processes of the eye, and its drainage through the trabecular meshwork. Aqueous humor flows from the ciliary processes into the posterior chamber, bounded posteriorly by the lens and the zonules of Zinn, and anteriorly by the iris. It then flows through the pupil of the iris into the anterior chamber, bounded posteriorly by the iris and anteriorly by the cornea. From here, the trabecular meshwork drains aqueous humor via Schlemm's canal into scleral plexuses and general blood circulation.

In open/wide-angle glaucoma, flow is reduced through the trabecular meshwork, due to the degeneration and obstruction of the trabecular meshwork, whose original function is to absorb the aqueous humor. Loss of aqueous humor absorption leads to increased resistance and thus a chronic, painless buildup of pressure in the eye. In close/narrow-angle, the iridocorneal angle is completely closed because of forward displacement of the final roll and root of the iris against the cornea, resulting in the inability of the aqueous fluid to flow from the posterior to the anterior chamber and then out of the trabecular network. This accumulation of aqueous humor causes an acute increase of pressure and pain.

In some embodiments, the methods described herein can be used to treat diabetic retinopathy, a complication of diabetes that causes damage that results from microvascular retinal changes. Small blood vessels, such as those in the eye, are especially vulnerable to poor blood sugar control. An over accumulation of glucose and/or fructose damages the tiny blood vessels in the retina. Hyperglycemia-induced pericyte death and thickening of the basement membrane lead to increased permeability of the vascular walls, which changes the formation of the blood-retinal barrier. In some individuals, diabetic retinopathy is accompanied by macular edema. As diabetic retinopathy progresses, the lack of oxygen in the retina causes fragile, new, blood vessels to grow along the retina and in the vitreous humour. Without timely treatment, these new blood vessels can bleed, cloud vision, and destroy the retina and/or cause tractional retinal detachment.

In certain embodiments, the methods described herein can be used to treat retinitis pigmentosa (RP), a group of inherited, degenerative eye diseases that cause severe vision impairment and blindness. Mutations in more than 60 genes are known to cause retinitis pigmentosa. Approximately 20% of RP is autosomal dominant (ADRP), 20% is autosomal recessive (ARRP), and 10% is X linked (XLRP), while the remaining 50% is found in patients without any known affected relatives. The genes associated with retinitis pigmentosa play essential roles in the structure and function of photoreceptors in the retina, and the progressive degeneration of these cells causes vision loss.

In certain embodiments, the methods described herein can be used to treat proliferative vitreoretinopathy, i.e., the formation of scar tissue within the eye that is often a complication of rhegmatogenous retinal detachment. During rhegmatogenous retinal detachment, fluid from the vitreous humor enters a retinal hole. The accumulation of fluid in the subretinal space and the tractional force of the vitreous on the retina result in rhegmatogenous retinal detachment. During this process the retinal cell layers come in contact with vitreous cytokines, which trigger the proliferation and migration of retinal pigmented epithelium (RPE). The RPE cells undergo epithelial-mesenchymal transition (EMT) and develop the ability to migrate out into the vitreous. During this process the RPE cell layer-neural retinal adhesion and RPE-ECM (extracellular matrix) adhesions are lost. The RPE cells lay down fibrotic membranes while they migrate and these membranes contract and pull at the retina, and this can lead to secondary retinal detachment after primary retinal detachment surgery.

In certain embodiments, the treatment methods described herein can be used in conjunction with, e.g., surgery for the repair of a retinal tear, hole or detachment, or with, e.g., radiation therapy for the treatment of ocular melanoma.

In certain embodiments, the compositions and methods described herein can be used to treat and/or improve the outcome of corneal wound healing and/or corneal transplantation. The corneal wound healing response is a complex cascade involving cytokine mediated interactions between the epithelial cells, stromal keratocytes, corneal nerves, lacrimal glands, tear film and cells of the immune system. The response of the tissue changes depends on the inciting injury. For example, incisional, lamellar and surface scrape injuries, like the ones used in keratorefractive surgery procedures, are followed by typical wound healing responses that are similar in some respects, but different in others. For example, elsewhere in the body, wound healing culminates in scar formation and vascularisation whereas one of the most crucial aspects of corneal wound healing is how the healing processes aim to minimize these end results, which would otherwise have serious visual consequences. Causes of corneal scarring include almost any disruption to normal corneal structure and function, whether from infection, laser refractive surgery, corneal transplantation, ocular trauma (chemical or physical) or corneal dystrophies.

Corneal transplantation, also known as corneal grafting, is a surgical procedure where a damaged or diseased cornea is replaced by donated corneal tissue (the graft) in its entirety (penetrating keratoplasty) or in part (lamellar keratoplasty). The graft is taken from a recently deceased individual with no known diseases or other factors that may affect the viability of the donated tissue or the health of the recipient. Since the cornea has no blood vessels (it takes its nutrients from the aqueous humor) it heals much more slowly than a cut on the skin. The risks are similar to other intraocular procedures, but additionally include graft rejection (lifelong), detachment or displacement of lamellar transplants and primary graft failure. There is also a risk of infection.

The present invention provides methods of treating an ocular disease described herein by administering an effective amount of a composition comprising a construct. In some embodiments, the invention provides methods of treating one or more aspects or symptoms of the ocular diseases described herein, including, but not limited to, formation of ocular drusen, inflammation in the eye or eye tissue, loss of photoreceptor cells, loss of vision (including for example visual acuity and visual field), neovascularization (such as choroidal neovascularization or CNV), and retinal detachment. Other related aspects, such as photoreceptor degeneration, RPE degeneration, retinal degeneration, chorioretinal degeneration, cone degeneration, retinal dysfunction, retinal damage in response to light exposure (such as constant light exposure), damage of the Bruch's membrane, loss of RPE function, gain or RPE function, loss of integrity of the histoarchitecture of the cells and/or extracellular matrix of the normal macular, loss of function of the cells in the macula, photoreceptor dystrophy, mucopolysaccharidoses, rod-cone dystrophies, cone-rod dystrophies, anterior and posterior uvitis, and diabetic neuropathy, are also included.

In some embodiments, there are provided methods of treating a drusen-associated disease. The term “drusen-associated disease” refers to any disease in which formation of drusen or drusen-like extracellular disease plaque takes place, and for which drusen or drusen-like extracellular disease plaque causes or contributes to thereto or represents a sign thereof. For example, AMD, characterized by the formation of macular drusen, is considered as a drusen-associated disease. Non-ocular drusen-related diseases include, but are not limited to, amyloidosis, elastosis, dense deposit disease, and/or atherosclerosis.

### Modes of Administration

The compositions described herein can be administered to an individual via any route, including, but not limited to, intravenous (e.g., by infusion pumps), intraperitoneal, intraocular, intra-arterial, intrapulmonary, oral, intravesicular, intramuscular, intra-tracheal, subcutaneous, intraocular, intrathecal, transdermal, transpleural, topical, inhalational (e.g., as mists of sprays), mucosal (such as via nasal mucosa), gastrointestinal, intraarticular, intracisternal, intraventricular, rectal (i.e., via suppository), vaginal (i.e., via pessary), intracranial, intraurethral, intrahepatic, and intratumoral. In some embodiments, the compositions are administered systemically (for example by intravenous injection). In some embodiments, the compositions are administered locally (for example by intraarterial or intraocular injection).

In some embodiments, the compositions are administered directly to the eye or the eye tissue. In some embodiments, the compositions are administered topically to the eye, for example, in eye drops. In some embodiments, the compositions are administered by injection to the eye (intraocular injection) or to the tissues associated with the eye. The compositions can be administered, for example, by intraocular injection, periocular injection, subretinal injection, intravitreal injection, trans-septal injection, subscleral injection, intrachoroidal injection, intracameral injection, subconjunctival injection, sub-Tenon's injection, retrobulbar injection, peribulbar injection, or posterior juxtascleral delivery. These methods are known in the art. For example, for a description of exemplary periocular routes for retinal drug delivery, see Periocular routes for retinal drug delivery, Raghava et al. (2004), Expert Opin. Drug Deliv. 1(1):99-114. The compositions may be administered, for example, to the vitreous, aqueous humor, sclera, conjunctiva, the area between the sclera and conjunctiva, the choroid tissues, macula, or other area in or proximate to the eye of an individual. The compositions can also be administered to the individual as an implant. Preferred implants are biocompatible and/or biodegradable sustained release formulations which gradually release the compounds over a period of time. Ocular implants for drug delivery are well-known in the art. See, e.g., U.S. Pat. Nos. 5,501,856, 5,476,511, and 6,331,313. The compositions can also be administered to the individual using iontophoresis, including, but are not limited to, the iontophoretic methods described in U.S. Pat. No. 4,454,151 and U.S. Pat. App. Pub. No. 2003/0181531 and 2004/0058313.

In some embodiments, the compositions are administered intravascularly, such as intravenously (IV) or intraarterially. In some embodiments (for example for the treatment of renal diseases), the compositions are administered directly into arteries (such as renal arteries).

In some embodiments, the compositions are administered directly into the joint tissue. In some embodiments, the compositions are administered to the synovium.

The optimal effective amount of the compositions can be determined empirically and will depend on the type and severity of the disease, route of administration, disease progression and health, mass and body area of the individual. Such determinations are within the skill of one in the art. The effective amount can also be determined based on in vitro complement activation assays. Examples of dosages of constructs which can be used for methods described herein include, but are not limited to, an effective amount within the dosage range of any of about 0.01 μg/kg to about 300 mg/kg, or within about 0.1 μg/kg to about 40 mg/kg, or with about 1 μg/kg to about 20 mg/kg, or within about 1 μg/kg to about 10 mg/kg. For example, when administered intraocularly, the composition may be administered at low microgram ranges, including for example about 0.1 μg/kg or less, about 0.05 μg/kg or less, or 0.01 μg/kg or less. In some embodiments, the amount of a construct administered to an individual is about 10 μg to about 500 mg per dose, including for example any of about 10 μg to about 50 μg, about 50 μg to about 100 μg, about 100 μg to about 200 μg, about 200 μg to about 300 μg, about 300 μg to about 500 μg, about 500 μg to about 1 mg, about 1 mg to about 10 mg, about 10 mg to about 50 mg, about 50 mg to about 100 mg, about 100 mg to about 200 mg, about 200 mg to about 300 mg, about 300 mg to about 400 mg, or about 400 mg to about 500 mg per dose.

The compositions may be administered in a single daily dose, or the total daily dose may be administered in divided dosages of two, three, or four times daily. The compositions can also be administered less frequently than daily, for example, six times a week, five times a week, four times a week, three times a week, twice a week, once a week, once every two weeks, once every three weeks, once every month, once every two months, once every three months, or once every six months. The compositions may also be administered in a sustained release formulation, such as in an implant which gradually releases the composition for use over a period of time, and which allows for the composition to be administered less frequently, such as once every month, once every 2-6 months, once every year, or even a single administration. The sustained release devices (such as pellets, nanoparticles, microparticles, nanospheres, microspheres, and the like) may be administered by injection or surgical implanted in various locations in the eye or tissue associated with the eye, such as intraocular, intravitreal, subretinal, periocular, subconjunctival, or sub-tenons.

The pharmaceutical compositions can be administered alone or in combination with other molecules known to have a beneficial effect on retinal attachment or damaged retinal tissue, including molecules capable of tissue repair and regeneration and/or inhibiting inflammation. Examples of useful cofactors include anti-VEGF agents (such as an antibody against VEGF), basic fibroblast growth factor (bFGF), ciliary neurotrophic factor (CNTF), axokine (a mutein of CNTF), leukemia inhibitory factor (LIF), neutrotrophin 3 (NT-3), neurotrophin-4 (NT-4), nerve growth factor (NGF), insulin-like growth factor II, prostaglandin E2, 30 kD survival factor, taurine, and vitamin A. Other useful cofactors include symptom-alleviating cofactors, including antiseptics, antibiotics, antiviral and antifungal agents and analgesics and anesthetics.

### Gene Therapy

The constructs can also be delivered by their expression in vivo, which is often referred to as “gene therapy”. For example, cells may be engineered with a polynucleotide (DNA or RNA) encoding for the construct ex vivo, the engineered cells are then provided to an individual to be treated with the fusion protein. Such methods are well-known in the art. For example, cells may be engineered by procedures known in the art by use of a retroviral particle containing RNA encoding for the fusion protein of the present invention.

Local delivery of the construct of the present invention using gene therapy may provide the therapeutic agent to the target area, for example to the eye or the eye tissue.

Methods of gene delivery are known in the art. These methods include, but are not limited to, direct DNA transfer, see, e.g., Wolff et al. (1990) Science 247: 1465-1468; 2) Liposome-mediated DNA transfer, see, e.g., Caplen et al. (1995) Nature Med. 3:39-46; Crystal (1995) Nature Med. 1:15-17; Gao and Huang (1991) Biochem. Biophys. Res. Comm. 179:280-285; 3) Retrovirus-mediated DNA transfer, see, e.g., Kay et al. (1993) Science 262:117-119; Anderson (1992) Science 256:808-813; 4) DNA Virus-mediated DNA transfer. Such DNA viruses include adenoviruses (preferably Ad2 or Ad5 based vectors), herpes viruses (preferably herpes simplex virus based vectors), and parvoviruses (preferably “defective” or non-autonomous parvovirus based vectors, more preferably adeno-associated virus based vectors, most preferably AAV-2 based vectors). See, e.g., Ali et al. (1994) Gene Therapy 1:367-384; U.S. Pat. No. 4,797,368, incorporated herein by reference, and U.S. Pat. No. 5,139,941.

Retroviruses from which the retroviral plasmid vectors described herein may be derived include, but are not limited to, Moloney Mouse Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus. In one embodiment, the retroviral plasmid vector is derived from Moloney Mouse Leukemia Virus.

Adenoviruses have the advantage that they have a broad host range, can infect quiescent or terminally differentiated cells, such as neurons or hepatocytes, and appear essentially non-oncogenic. See, e.g., Ali et al. (1994), supra, p. 367. Adenoviruses do not appear to integrate into the host genome. Because they exist extrachromosomally, the risk of insertional mutagenesis is greatly reduced. Ali et al. (1994), supra, p. 373.

Adeno-associated viruses exhibit similar advantages as adenoviral-based vectors. However, AAVs exhibit site-specific integration on human chromosome 19 (Ali et al. (1994), supra, p. 377).

The gene therapy vectors include one or more promoters. In some embodiments, the vector has a promoter that drives expression in multiple cell types. In some embodiments, the vector has a promoter that drives expression in specific cell types (such as cells of retina or cells in the kidney). Suitable promoters which may be employed include, but are not limited to, the retroviral LTR; the SV40 promoter; and the human cytomegalovirus (CVM) promoter described in Miller et al. (1989) Biotechniques 7(9):980-990, or any other promoter (e.g., cellular promoters such as eukaryotic cellular promoters including, but not limited to, the histone, pol III, and .beta.-actin promoters). Other viral promoters which may be employed include, but are not limited to, adenovirus promoters, thymidine kinase (TK) promoters, and B19 parvovirus promoters. The selection of a suitable promoter will be apparent to those skilled in the art from the teachings contained herein.

The nucleic acid sequence encoding a construct is under the control of a suitable promoter. Suitable promoters which may be employed include, but are not limited to, adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoA1 promoter; human globin promoters; viral thymidine kinase promoters, such as the Herpes Simplex thymidine kinase promoter; retroviral LTRs (including the modified retroviral LTRs hereinabove described); the 3-actin promoter; and human growth hormone promoter.

Retroviral plasmid vectors can be employed to transduce packaging cell lines to form producer cell lines. Examples of packaging cells which maybe transfected are described in Miller (1990) Human Gene Therapy 1:5-14. The vectors may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host. The producer cell line generates infectious retroviral vector particles which include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

In some embodiments, gene delivery vectors which direct expression of a construct in the eye are used. Vectors for gene delivery to the eye are known in the art, and have been disclosed, for example, in U.S. Pat. No. 6,943,153, and U.S. Patent Application Publication Nos. US20020194630, US20030129164, US200600627165.

In some embodiments, the complement activation is inhibited by contacting a body fluid with a composition comprising a construct ex vivo under conditions that permit the construct to function to inhibit complement activation. Suitable body fluids include those that can be returned to the individual, such as blood, plasma, or lymph. Affinity adsorption apheresis is described generally in Nilsson et al. (1988) Blood 58(1):38-44; Christie et al. (1993) Transfusion 33:234-242; Richter et al. (1997) ASAIO J. 43(1):53-59; Suzuki et al. (1994) Autoimmunity 19: 105-112; U.S. Pat. No. 5,733,254; Richter et al. (1993) Metabol. Clin. Exp. 42:888-894; and Wallukat et al. (1996) Int'l J. Card. 54:1910195.

Accordingly, the invention include methods of treating one or more diseases described herein in an individual comprising treating the individual's blood extracorporeally (i.e., outside the body or ex vivo) with a composition comprising a construct under conditions that permit the molecule to function to inhibit complement activation, and returning the blood to the individual.

### Unit Dosages, Articles of Manufacture, and Kits

Also provided are unit dosage forms of construct compositions, each dosage containing from about 0.01 mg to about 50 mg, including for example any of about 0.1 mg to about 50 mg, about 1 mg to about 50 mg, about 5 mg to about 40 mg, about 10 mg to about 20 mg, or about 15 mg of the construct. In some embodiments, the unit dosage forms of a construct composition comprises about any of 0.01 mg-0.1 mg, 0.1 mg-0.2 mg, 0.2 mg-0.25 mg, 0.25 mg-0.3 mg, 0.3 mg-0.35 mg, 0.35 mg-0.4 mg, 0.4 mg-0.5 mg, 0.5 mg-1.0 mg, 10 mg-20 mg, 20 mg-50 mg, 50 mg-80 mg, 80 mg-100 mg, 100 mg-150 mg, 150 mg-200 mg, 200 mg-250 mg, 250 mg-300 mg, 300 mg-400 mg, or 400 mg-500 mg construct. In some embodiments, the unit dosage form comprises about 0.25 mg construct. The term “unit dosage form” refers to a physically discrete unit suitable as unitary dosages for an individual, each unit containing a predetermined quantity of active material calculated to produce the desired therapeutic effect, in association with a suitable pharmaceutical carrier, diluent, or excipient. These unit dosage forms can be stored in a suitable packaging in single or multiple unit dosages and may also be further sterilized and sealed.

Also provided are articles of manufacture comprising the compositions described herein in suitable packaging. Suitable packaging for compositions (such as ophthalmic compositions) described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The present invention also provides kits comprising compositions (or unit dosages forms and/or articles of manufacture) described herein and may further comprise instruction(s) on methods of using the composition, such as uses described herein. The kits described herein may further include other materials desirable from a commercial and user standpoint, including other buffers, diluents, filters, needles, syringes, and package inserts with instructions for performing any methods described herein.

### EXAMPLES

### Example 1: Essential Role for the Lectin Pathway in Collagen Antibody-Induced Arthritis Revealed Through Use of Adenovirus Programming

### Materials and Methods

### Mice

Eight to ten week-old WT C57BL/6 male mice (n=73) were used for this study. We obtained C4−/− mice originally from Dr. Michael Carroll and C1q/MBL−/− mice from Dr. Gregory Stahl. Our laboratory has now maintained colonies of both C4−/− and C1q/MBL−/− C57BL/6 homozygous mice; sera from these mice were used for various ELISAs. WT C57BL/6 mice were obtained from Jackson Laboratories. All mice were weighed prior to use and were kept in a barrier animal facility with a climate-controlled environment with 12 h light/dark cycles. Filter top cages were used with three mice in each cage. During the course of this study, all experimental mice were fed breeder's chow provided by the Center for Laboratory Animal Care, University of Colorado School of Medicine.

### Construction of AdMAp44 Vectors

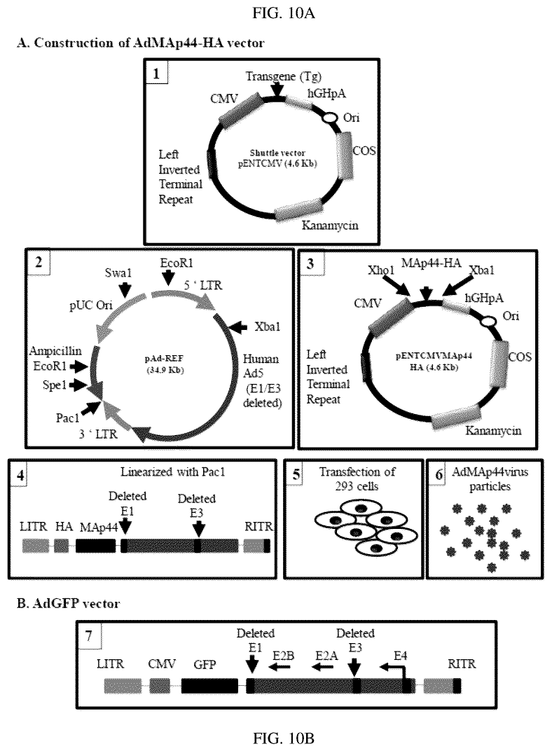

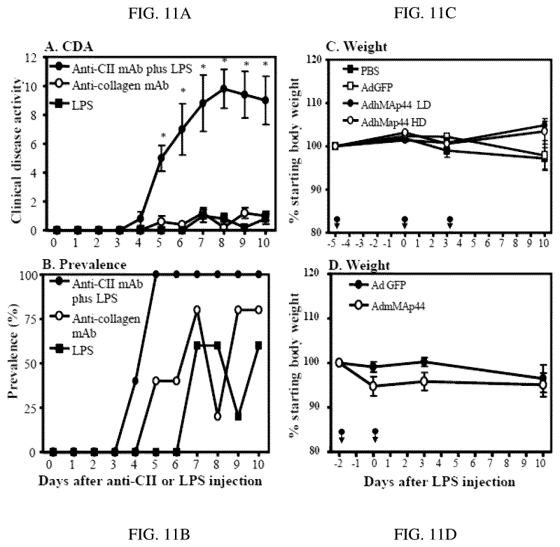

Human AdMAp44 (AdhMAp44) construct was generated by Welgen, Inc (Worcester, MA) using the human MAp44 cDNA purchased from Thermo Fisher (Waltham, MA). The HA-Tag (Human influenza hemagglutinin, sequence “YPYDVPDYA”) was added to the C-terminus of the MAp44 to facilitate the detection of recombinant MAp44 in the circulation of mice generated by the administration of AdhMAp44 or AdmMAp44. To detect the presence of HA in the sera of mice with and without CAIA, anti-HA tag antibodies were used. Information about the vectors and specific elements described herein is found in FIG. 10. Additional RGD sequences were added to the construct for AdhMAp44, but not AdmMAp44, to stimulate receptors for adenoviral entry in synovial cells other than the CAR (Bakker et al., 2001, Gene Ther. 8: 1785-1793). Briefly, pBSK-MAp-1-HA was cleaved with Xhol/Xbal and the MAp44-HA fragment was ligated to the pEntCMV shuttle vector digested with the same enzymes. Positive clones were screened and sequenced for confirmation. pEntCMV-MAp44-HA was treated with LR Clonase II (Invitrogen) and ligated with the plasmid pAd5. The recombination products were used to transform E. coli. After incubation overnight, clones were selected and grown, and cosmid DNA was purified. The purified cosmid DNA (2 mg) was digested with Pac and then transfected into 293 cells with Lipofectamine 2000 (Life Technologies) according to the manufacturer's instructions. The 293 cells were grown at 37° C. with 5% CO2. Ad plaque growth was apparent by 7 days after transfection. The titer of virus particles (vp) was further amplified to 10¹² vp/ml. The amplified Ad was purified on 2 sequential cesium chloride gradients and then dialyzed against PBS, pH 7.4, containing 10% glycerol. The titer of the purified virus was estimated from the absorption at 260 nm. The final titer of AdhMAp44 was 1.0×10¹² particles per ml. Ad with cytomegalovirus (CMV) sequences and programming expression of green fluorescent protein (GFP) (AdGFP) was used as a negative control for all CAIA studies.

### Human Recombinant MAp44

Human recombinant MAp44 (hrMAp44) was produced as described in detail elsewhere (Degn et al., 2009, J. Immunol. 183: 7371-7378). In brief mammalian cells, HEK293F cells, were transfected with a vector encoding human MAp44 using PEI as transfection reagent. MAp44 expressed in the supernatants was purified by affinity chromatography on MBL coated beads.

### Induction of Collagen Antibody-Induced Arthritis

CAIA was induced in WT mice using a cocktail of 5 mAbs to bovine CII (Arthritomab-CIA, Chondrex) suspended in sterile PBS as previously described (Banda et al., 2006, J. Immunol. 177: 1904-1912; Banda et al., 2007, J. Immunol. 179: 4101-4109; Banda et al., 2010, J. Immunol. 185: 5598-5606). WT mice were injected i.p. with 4 mg/mouse of Arthritomab on day 0 and 50 μg/mouse of LPS from E. coli strain 0111B4 (Chondrex) on day 3 to synchronize the development of arthritis according to the standard protocol suggested by the supplier of Arthritomab and LPS. Mice started to develop arthritis at day 4 and were sacrificed at day 10. Mice do develop very mild transient arthritis and with no histological damage (data not shown) for a few days when anti-CII abs or LPS were injected i.p. alone with 6 mg/mouse/i.p or 50 μg/mouse/i.p. respectively (FIG. 11A). Furthermore mice injected with anti-CII abs or LPS alone developed inconsistent disease as evident from prevalence and it is difficult to assess the cause and effect relationship of complement inhibitors (FIG. 11B). Therefore, an injection of anti-collagen mAbs followed by an injection of LPS is required for a sustained production of disease for a minimum of 10 days with histological damage and to observe the effects of complement inhibitors (FIG. 11A). Clinical disease activity (CDA) was examined daily until day 10 by observers blinded to the treatment as per our previously published studies (Banda et al., 2006, J. Immunol. 177: 1904-1912; Banda et al., 2007, J. Immunol. 179: 4101-4109; Banda et al., 2010, J. Immunol. 185: 5598-5606).

In Ad studies, WT mice were injected i.p. at day −5, day 0, and day 3 with AdhMAp44 (at either a higher dose (1×10¹¹) or a lower dose (5.0×10¹⁰)), AdGFP (1×10¹¹ particles), or PBS alone (n=5 for each treatment). Arthritomab was injected as usual at day 0. For the local joint injection experiment, 50 μl containing 5.0×10¹⁰ particles of AdmMAp44 or of AdGFP were injected in the right knee joint at day −5, day 0 (after the anti-CII mAb injection), and at day 3 (after LPS injection).

### Ross River Virus-Induced Mouse Model of Inflammatory Arthritis

Three-to-four week old WT C57BL/6 mice were inoculated in the left rear footpad with 103 PFU of Ross River virus (RRV) in a volume of 10 μl as previously described (Morrison et al., 2007, J. Virol. 81: 5132-5143; Morrison et al., 2008, J. Virol. 82: 11263-11272; Morrison et al., 2006, J. Virol. 80: 737-749). Disease scores were determined by assessing grip strength, hind limb weakness, and altered gait as previously described (Morrison et al., 2007, J. Virol. 81: 5132-5143; Morrison et al., 2008, J. Virol. 82: 11263-11272; Morrison et al., 2006, J. Virol. 80: 737-749). In RRV-induced arthritis, AdhMAp44 and AdGFP were injected with doses of viral particles identical to those used in the CAIA studies for knee injection, i.e., 5×10¹⁰ at days −3, 0, and 3 in the right rear footpad.

### Histopathology and Immunohistochemistry of all Joints

Knee joints from both fore limbs, and the right hind limb knee joint, ankle and paw from WT mice with CAIA at day 10 were fixed in 4% paraformaldehyde and examined by immunohistochemical staining (IHS) for Toluidine-blue (T-blue) and C3 deposition according to our published methods (Banda et al., 2006, J. Immunol. 177: 1904-1912; Banda et al., 2007, J. Immunol. 179: 4101-4109; Banda et al., 2010, Clin. Exp. Immunol. 159: 100-108). Additionally, IHS was used to detect integrin αvβ5 (antibody dilution 1:500) in the synovium and knee joint sections from mice transduced with AdhMAp44 and AdGFP. Hemotoxylin (VWR) staining was used to show the presence of the synovium. Toluidine-blue stain was used to assess histopathology for determination of inflammation, pannus formation, and cartilage and bone damage according to published criteria (Banda et al., 2006, J. Immunol. 177: 1904-1912; Banda et al., 2007, J. Immunol. 179: 4101-4109; Banda et al., 2010, Clin. Exp. Immunol. 159: 100-108). Seven m sections were cut for histology and processed for T-blue and C3 IHS. All slides for histopathology and C3 deposition were observed under light microscopy at a magnification of 20× or 10× in a blinded fashion and scored according to published criteria (Banda et al., 2006, J. Immunol. 177: 1904-1912; Banda et al., 2007, J. Immunol. 179: 4101-4109; Banda et al., 2010, Clin. Exp. Immunol. 159: 100-108). The knee joints from untreated C3−/− mice on C57BL/6 background were used as negative controls.

### Immunohistochemistry to Detect GFP and the HA Tag in Organs

Knee joints from the right hind limb, liver, spleen and kidney from WT mice with and without CAIA at day 10 were fixed in 4% paraformaldehyde and examined by IHS for GFP. Anti-GFP polyclonal rabbit (dilution 1:200) and the secondary antibody (goat anti-rabbit, Alexa Flour 488 (1:200 dilution) (Invitrogen)) were used to detect GFP. Sections were visualized under UV-light using an Olympus (Model—BX51) microscope. The presence of green fluorescence under UV-light indicated the presence of GFP expression in tissues.

Additionally, we examined for the presence of HA in the synovium from the knee joints of mice injected i.p. with AdhMAp44 and AdmMAp44 at day 10. At the time of sacrifice liver, spleen, kidney and knee joints were collected and fixed in 10% neutral-buffered formalin, processed and sections were cut. After staining with anti-HA antibody (dilution 1:1000) (Cell Signal), and the development of color using anti-Rabbit En Vision plus Polymer HRP-conjugated followed by DAB plus Chromogen (Dako), the sections were visualized by light microscopy and photographed.

### Examination of In Vivo Transduction Efficiency and Western Blot Analysis

Since both AdhMAp44 and AdmMAp44 constructs utilized a HA tag, we analyzed sera at days −5 or −2, 0, 3 and 10 after the i.p. injections for the presence of HA using Western blot analysis. Similarly, we analyzed the sera from mice injected in the knee joints with AdmMAp44 at days −5, 0, 3 and 10 for the presence of HA using Western blot analysis.

### Western Blot Analysis

A 10% Bis-Tris reducing SDS gel was used for separation of proteins in mouse serum. After transfer, the blots were incubated overnight at 40 C with rabbit Ab specific for HA (dilution 1:1000) (Cell Signal). Anti-rabbit HRP-conjugated Ab was used as the secondary Ab (dilution 1:2000) (Hycult Biotech). The blots were washed 3×10 min in 1×PBS 0.5% Tween 20 and developed for 3 min using a 1:1 mixture of SuperSignal West Pico chemiluminescent substrate (Thermo Scientific). The presence of HA and MAp44 bands at ˜43-50 kDa in serum identified the presence in circulation of AdhMAp44 or AdmMAp4, indicating successful synthesis and secretion. We also evaluated whether the expressed hMAp44 protein was functionally active, i.e. did it bind to MBL. This was examined by incubation of sera with mannose-agarose beads, which bind MBL and, thus, should indirectly bind MAp44 through its interaction with MBL. The material eluted from the beads was analyzed by Western blot analysis and probed with anti-HA Ab as noted above.

Quantitative RT-PCR for mRNA Expression Levels

Knee joints were harvested from mice with CAIA at day 10 after induction. Total RNA was extracted using an RNAeasy mini kit (Qiagen) from the left knee joints of all experimental mice injected i.p. with PBS, AdhMAp44 LD, AdhMAp44 HD, or AdGFP at days −5, 0, and 3. The presence of mouse MBL-A, MBL-C, Ficolin-A (FCN-A), MASP-1, MASP-2, MASP-3, FD, TNF-α, IL-1α and IL-1β were analyzed in the samples by RT-PCR using 40 cycles according to published methods (Schmittgen and Livak, 2008, Nat. Protoc. 3: 1101-1108). All RT-PCR data were analyzed using a cDNA based standard curve. The standard curves for mRNA encoding FD, MASP-1, MASP-2, and MASP-3 were constructed by using mRNA from mouse adipose tissue for FD and liver for MBL-A, MBL-C, FCN-A, TNF-α, IL-1α and IL-1β, and the MASPs, respectively. In parallel, the baseline mRNA levels for various targets from knee joint of age-matched WT mice without CAIA and without any treatment were also determined. Primer sequences used to determine mRNA concentrations are available upon request from the corresponding authors.

### Human MAp44 Assay

A sandwich type immunoassay method was used to determine the absolute levels of human MAp44 present in the circulation of WT mice injected with PBS, AdGFP or AdhMAp44 (low dose or high dose) according to recently published studies (Degn et al., 2010, J. Immunol. Methods 361: 37-50). This assay is highly specific and sensitive for human MAp44 and can be used to determine the levels of human MAp44 in mouse serum. To measure the levels of MAp44, serum from each mouse obtained at day −5, day 0, day 3 and at day 10 was diluted 1:15 in a binding buffer. A standard human plasma pool with a known level of human MAp44 was used to establish a standard curve. All samples were tested in duplicate, and three quality controls were included in each assay as described (Degn et al., 2010, J. Immunol. Methods 361: 37-50).

### Measurements of Absolute Levels of C5a in Serum

Serum levels of C5a before (day −5) and after (day 10) the development of disease in WT mice injected with AdhMAp44 or AdGFP or PBS were measured using standard ELISA protocols according to our published methods (Banda et al., 2012, J. Immunol. 188: 1469-1478).

### Measurements of LP Induced C3 in Serum

LP induced C3 activation using sera from CAIA mice, at day 10, treated with PBS or AdGFP or AdhMAp44 LD or AdhMAp44 HD was determined by using mannan particles pre-coated ELISA plates according to previously described methods (Banda and Takahashi, 2014, Methods in molecular biology 1100: 365-371). Sera from WT mice with no CAIA were used as a positive control. Sera from C3−/− and MBUDf−/− mice were used as a negative controls.

### Analysis of Recombinant Human MAp44 on LPS Induced C3 Activation

The effect of recombinant human MAp44 (rhMAp44) on LPS-induced C3 activation via LP and AP was determined by using ELISA. A 96-well ELISA plates were pre-coated with 5 μg/well of LPS from E. coli strain 0111B4. Serum from WT mouse with no disease was diluted (1:10) and pre-treated for 30 minutes with rhMAp44 (10 μg/10 μl of serum) in GBV+ buffer (Ca⁺⁺-sufficient buffer) or in Mg²⁺ EGTA buffer (Ca⁺⁺-deficient buffer). LPS induced C3 activation was measured according to the methods described by (Kimura et al., 2008, Blood 111: 732-740). In parallel serum from WT mouse was also pre-treated with an inhibitory anti-factor B antibody (4 μg/10 μl of serum) as a positive control to inhibit C3 activation specifically from the AP. Serum from C3−/− mice was used as a negative control and there was no C3 activation as expected.

### Statistical Analyses

Student's t test was used to calculate p-values using the GraphPad Prism® 4 statistical program. The data in all graphs and histograms are shown as the mean+SEM with p<0.05 considered significant. Pearson correlation was used to calculate r² value among histological parameters, C3 deposition and CDA. Preliminary analyses using a null hypothesis for w-statistics indicated that the data were normally distributed.

### Results

Human AdMAp44 Prevents Initiation of Disease in Mice with CAIA

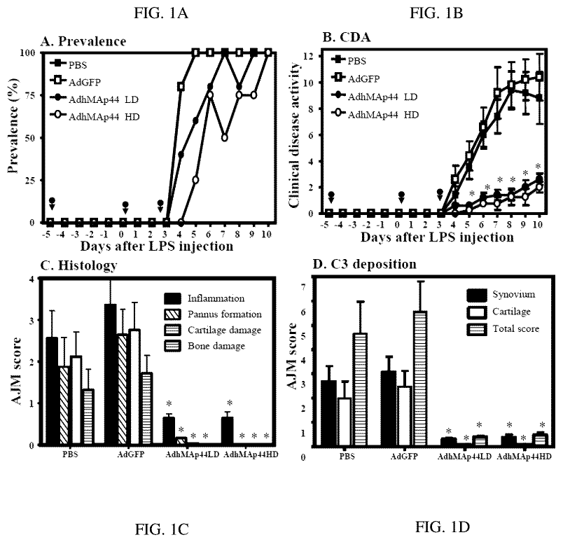

The generation of AdGFP, AdhMAp44, and AdmMAp44 is described in detail in Materials and Methods and illustrated in FIG. 10. To determine the effect of human MAp44 expression, we examined the development of CAIA in WT mice treated with a higher and lower dose of AdhMAp44 as compared to AdGFP and PBS buffer alone. The mice were treated with AdhMAp44, AdGFP or PBS alone on days −5, 0, and 3. Anti-CII mAb was injected i.p. on day 0. The prevalence of disease in each condition was 100% at day 10 (FIG. 1A). The CDA in WT mice injected with a higher (HD) or lower dose (LD) of AdhMAp44 was significantly reduced by 81% and 75%, respectively, as compared to WT mice injected with an equivalent higher dose of AdGFP (FIG. 1B). Specifically, the CDA at day 10 in mice treated with the HD and LD of AdhMAp44 was 2.0+0.4 and 2.6+0.4, respectively. In contrast, in mice treated with PBS and AdGFP, the CDA at day 10 was 10.6+1.8 and 8.8+2.0, respectively. These results demonstrate that while pretreatment with either dose of AdhMAp44 did not prevent CAIA, it significantly reduced the severity.

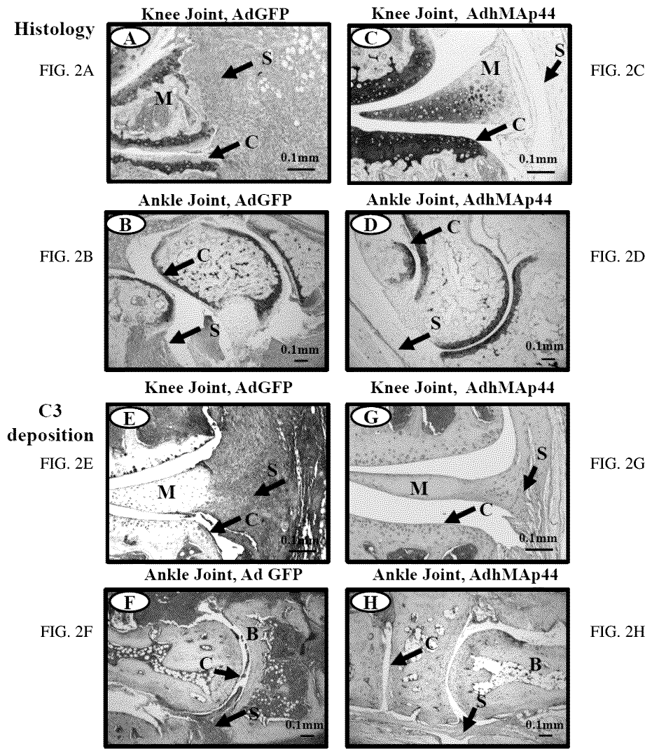

A significant decrease (p<0.034) was seen in the total histopathology score as well as in the individual scores for inflammation (p<0.0080), pannus (p<0.006), cartilage damage (p<0.007), and bone damage (p<0.009) in mice treated with a HD of AdhMAp44 in comparison to mice treated with AdGFP (FIG. 1C). Almost identical results were observed in mice treated with a LD of AdhMAp44 (FIG. 1C). The correlation coefficient (r2) between CDA and histology scores was highly positive (0.99). Representative tissue sections of histology are shown in knee joints (FIG. 2A, C) and ankle (FIG. 2B, D) of mice injected i.p. with AdGFP or AdhMAp44 (HD), respectively.

The level of C3 deposition in the synovium and cartilage was also significantly decreased in mice injected with either dose of AdhMAp44 in comparison to the mice injected with AdGFP or PBS (FIG. 1D). A significant decrease was seen in the total C3 deposition score as well as in the individual scores for synovium (p<0.001) and cartilage (p<0.003) in mice treated with a LD of AdhMAp44 in comparison to mice treated with AdGFP (FIG. 1D). Almost identical results were seen in mice treated with a HD of AdhMAp44 (FIG. 1D). Overall there was more than a 90% decrease in C3 deposition in the knee joint of mice either treated with a LD or with a HD of AdhMAp44. Representative tissue sections of C3 deposition are shown in knee joints (FIG. 2E, G) and ankle joints (FIG. 2F, H) of mice injected i.p. with AdGFP or AdhMAp44 (HD), respectively. There was no effect on the weight of mice before, during and after the development of disease in mice treated with a LD or HD of AdhMAp44 as compared to AdGFP or PBS (FIG. 11C).

### Effect of AdhMAp44 on C5a Levels in Serum

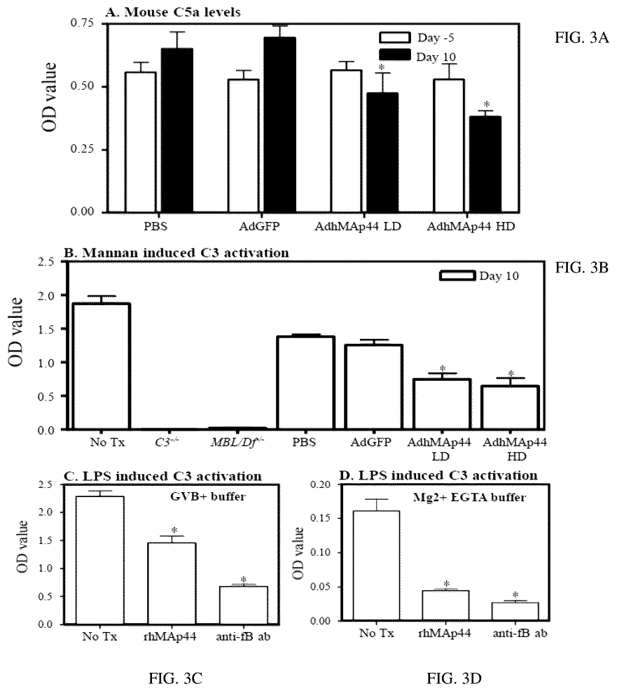

A 22% (p<0.045) and 45% (p<0.001) decrease in the level of C5a at day 10 was noted using sera from mice treated with AdhMAp44 using a LD or a HD, respectively, as compared to AdGFP-transduced mice (FIG. 3A). At day −5, i.e. prior to treatment, the absolute levels of C5a were identical and there was no significant difference as expected in all treatment groups. The decrease in the absolute levels of C5a in serum of CAIA were consistent with the anticipated effect in the model of AdMAp44 treatment on complement activation.

### Effect of AdhMAp44 on Mannan-Induced C3 Activation

Mannan particles specifically activate C3 through the LP. There was a 40% and 49% decrease at day 10 in the C3 activation induced by sera from mice treated with AdhMAp44 LD and AdhMAp44 HD vs. AdGFP, respectively (FIG. 3B). The O.D. values for WT serum were 1.187±0.108, PBS 1.383±0.035, AdGFP 1.258±0.078, AdhMAp44 LD 0.750±0.086 (p<0.002 vs. AdGFP), and AdhMAp44 HD 0.646±0.122 (p<0.003 vs. Ad GFP). In contrast, no decrease in C3 activation was seen in the sera from mice treated with PBS or AdGFP alone. There was no C3 activation using sera from C3−/− and MBUDf−/− mice as expected. Sera from C3−/− and MBUDf−/− mice were used as negative controls for ELISA. These results show that recombinant human MAp44 present in the circulation of CAIA mice affected activation of the LP.

### Effect of Recombinant Human MAp44 on LPS-Induced C3 Deposition

LPS is known to activate complement through both the LP and AP. LPS is used in our disease model of CAIA after the passive infusion of anti-CII mAbs, although the mechanism whereby LPS enhances disease is unknown. The possibility exists that rhMAp44 may inhibit the enhancing effects of LPS on the initiation of CAIA through the LP or AP of the complement system. We examined this question through induction of C3 deposition in vitro using LPS-coated plates and serum in the presence of Ca⁺⁺-sufficient buffer, enabling all 3 complement pathways, or Ca⁺⁺-deficient buffer with Mg⁺⁺ EGTA where only the AP is active. LPS-induced C3 deposition in the presence of Ca⁺⁺ was decreased from 2.28±0.10 O.D. to 1.45±0.13 O.D. in the absence or presence of rhMAp44, a 36% reduction (p<0.002), and decreased to 0.679±0.042 in the presence of anti-FB mAb, a 70% reduction (p<0.001) (FIG. 2C). More marked results were observed in Ca⁺⁺-deficient buffer where the O.D. values for LPS-induced C3 deposition using no treatment were 0.161±0.017, with rhMAp44 were 0.044±0.003 (p<0.0004), and with anti-FB mAb were 0.027±0.003 (p<0.0002) (FIG. 2D); these decreases were 72% and 83%, respectively. These results suggest that rhMAp44 may inhibit the induction of complement primarily through the AP.

Human MAp44 is Present in the Circulation of Mice after Injection of AdhMAp44

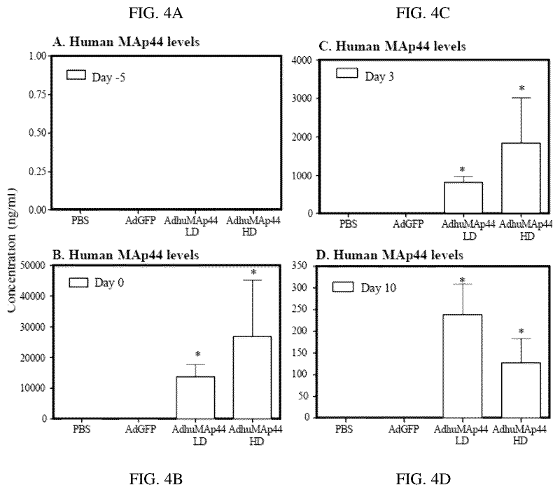

Using an ELISA human MAp44 was found to be present in the sera at day −5, day 0, day 3 and at day 10 from mice with CAIA treated with a LD or HD of AdhMAp44, but not in the sera from mice injected with PBS or AdGFP alone (FIG. 4A-D). At day 0, a huge highly significant increase (P<0.001) in the levels of human MAp44 was seen in the circulation of mice treated with LD or HD dose AdhMAp44 (FIG. 4). At day 3, the levels of human MAP44 were 808.6±170.54 (ng/ml) and 1841.0±1173.9 (ng/ml) in the circulation of mice treated with a LD or HD of AdhMAp44, respectively (FIG. 4C). At day 10, the levels of human MAp44 were 238.39±70.66 (ng/ml) and 127.25±56.08 (ng/ml) in the circulation of mice treated with a LD or HD of AdhMAp44, respectively (FIG. 4D). The differences in the level of human MAp44 between a LD and HD of AdhMAp44, at day 10, were not statistically significant (p>0.5). As expected no human MAp44 was detected using sera from WT mice without any treatment (data not shown). The presence of human MAp44 in the circulation at day 0, at day 3 and at day 10 of mice demonstrates that Ad-targeted cells were effectively transduced with AdhMAp44 to produce human MAp44.

AdmMAp44 Treatment Also Substantially Prevents Clinical Disease Activity in Mice with CAIA

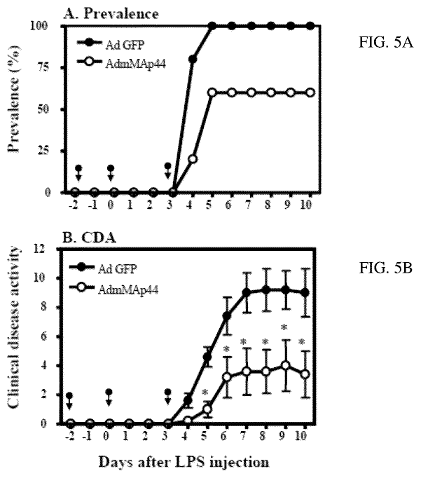

To confirm that the effect of AdhMAp44 was not due to non-physiologic effects of the human protein in mouse, the effects of expression of mouse MAp44 were also examined. To evaluate this question, mice were injected i.p. with AdmMAp44 and control AdGFP as described in Materials and Methods. At day 10 the CDA in WT mice injected with AdGFP and AdmMAp44 was 9.0+1.65 and 3.4+1.60, respectively (FIG. 5B). Thus, at day 10 CDA was reduced by 60% (p<0.026) in mice pre-treated with AdmMAp44 as compared to mice pre-treated identically with AdGFP. The prevalence of disease at day 10 in WT mice injected with AdGFP or AdmMAp44 was 100% and 60%, respectively (FIG. 5A). There was no significant effect on the weight of mice treated with AdGFP as compared to AdmMAp44 (FIG. 11D). These data demonstrate that, similarly to AdhMAp44, treatment with AdmMAp44 prevents the development of CAIA.

### Exogenous Mouse MAp44 Expression Prevents Histological Changes and C3 Deposition in the Joints in CAIA

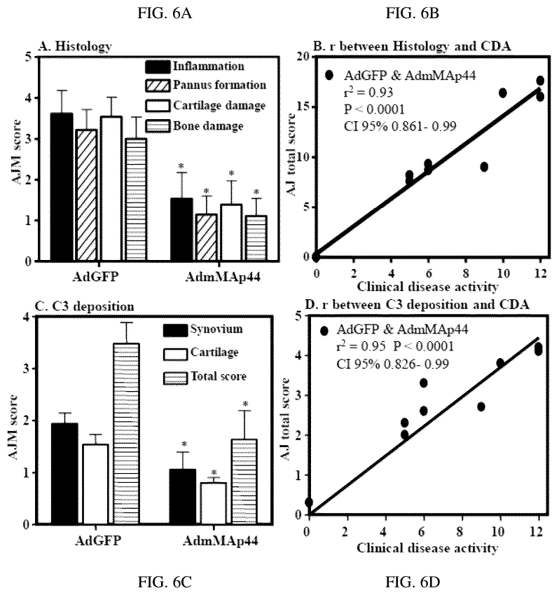

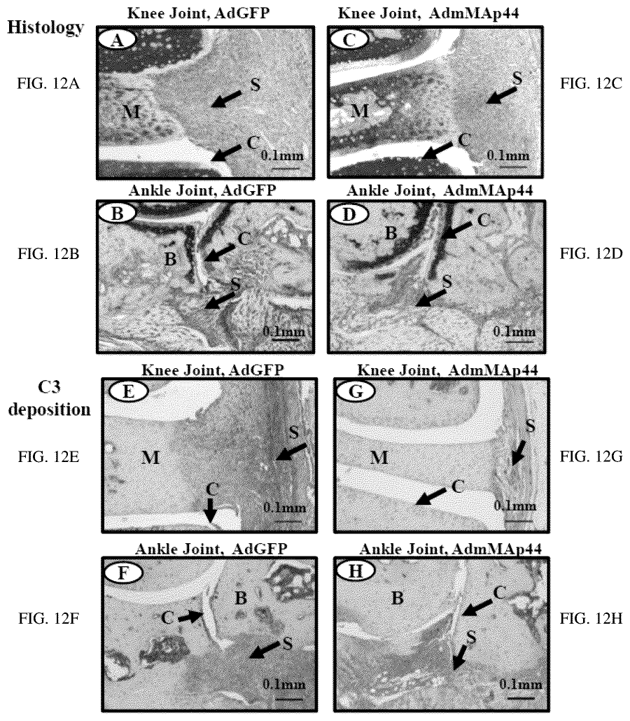

To further examine the treatment effect of AdmMAp44, histopathologic analyses were performed in fixed joints from mice injected i.p. with AdGFP or with AdmMAp44. A significant decrease (p<0.023) was seen in the total histopathology score as well as in the individual scores for inflammation (p<0.040), pannus (p<0.015), cartilage damage (p<0.021), and bone damage (p<0.026) in mice treated with AdmMAp44 as compared to AdGFP (FIG. 6A). The correlation coefficient (r2) between CDA and histology scores was 0.93 (FIG. 6B). The levels of C3 deposition in the synovium and cartilage were also significantly decreased in mice transduced with AdmMAp44 in comparison to transduction with AdGFP (p<0.02) (FIG. 6C). The correlation coefficient (r2) between CDA and C3 deposition scores was highly positive (0.95) (FIG. 6D). Representative tissue sections are shown in knee joints (FIG. 12A, C) and ankle (FIG. 12B, D) of mice injected i.p. with AdGFP or AdmMAp44. Representative tissue sections of C3 deposition are shown in knee joints (FIG. 12E, G) and ankle (FIG. 12F, H) of mice injected with AdGFP or AdmMAp44.

Systemic Effects of AdmMAp44 Injected into the Right Knee Joint

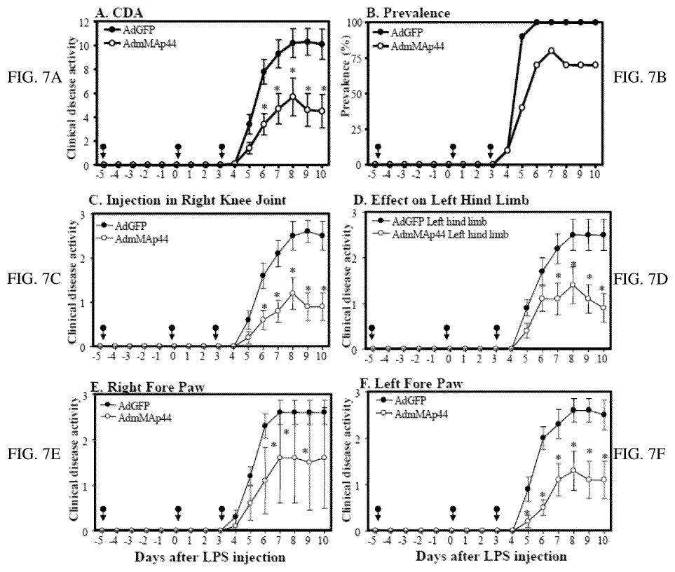

AdmMAp44 or AdGFP were injected three times in the right knee during the development of CAIA at days −5, 0, and 3 (with anti-CII mAb injected at day 0); the un-injected left knee served as a control. The overall CDA in all of the indicated joints in mice with CAIA pretreated with AdmMAp44 was significantly reduced by 55% as compared to mice injected with AdGFP; the CDA values were AdGFP 10.1+1.26 and AdmMAp44 4.5+1.40, respectively (p<0.008) (FIG. 7A). The prevalence of disease at day 10 in CAIA mice pretreated with AdGFP or AdmMAp44 was 100% and 70%, respectively (FIG. 7B). Injection in the right knee joint led to a decreased CDA in this joint (FIG. 7C). Following injection in the right knee joint, decreases in CDA were also observed in the left hind limb (FIG. 7D), right forepaw (FIG. 7E), and left forepaw (FIG. 7F). This experiment was repeated two times with identical injection schedules in the right knee, and the data were pooled. Thus, in addition to demonstrating that AdmMAp44 prevents CAIA in mice within the locally injected right knee joint, the effect was systemic because the CDA in all joints was substantially diminished.

### AdhMAp44 Treatment Decreases Severity of Ross River Virus-Induced Arthritis and Myositis

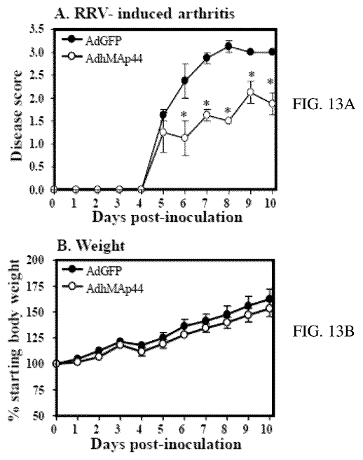

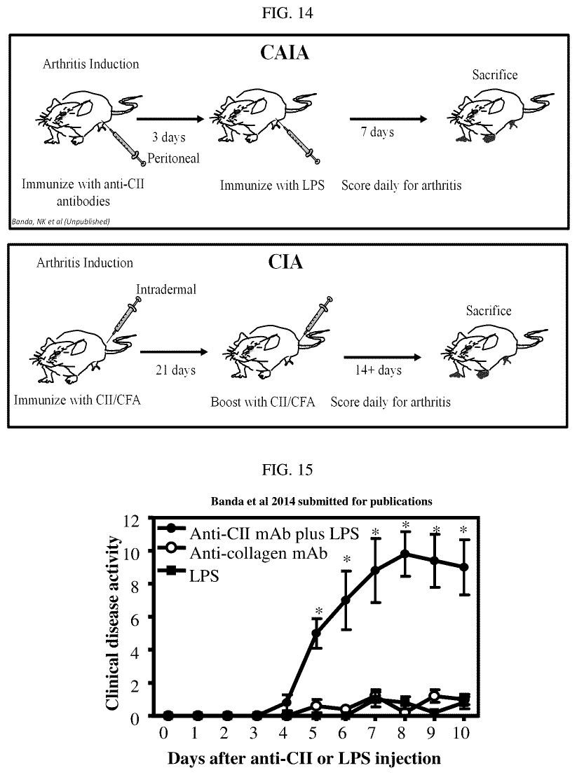

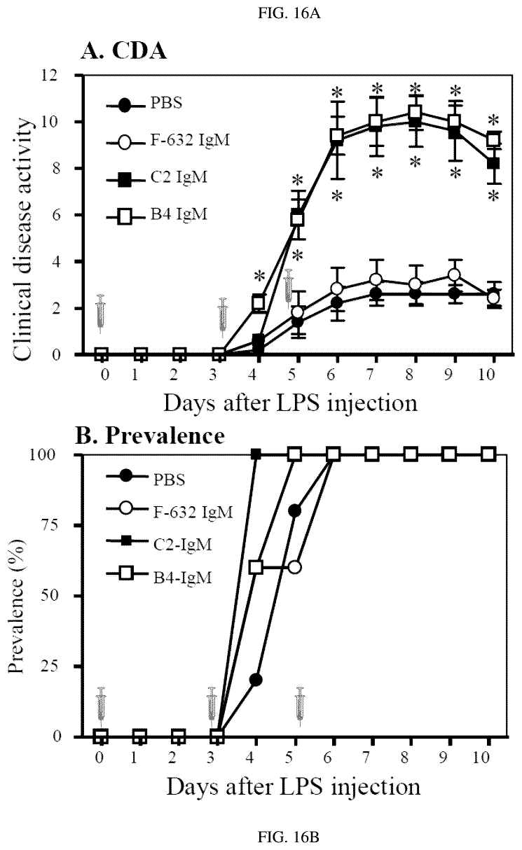

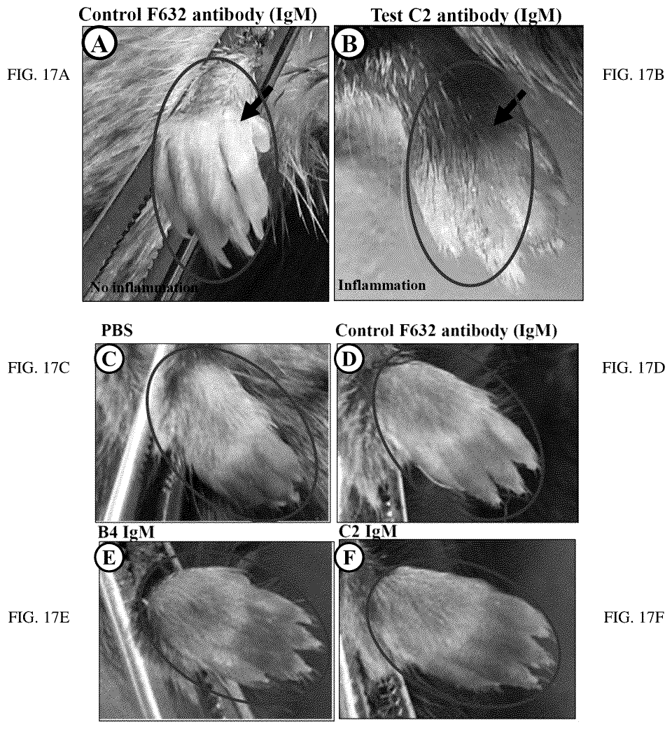

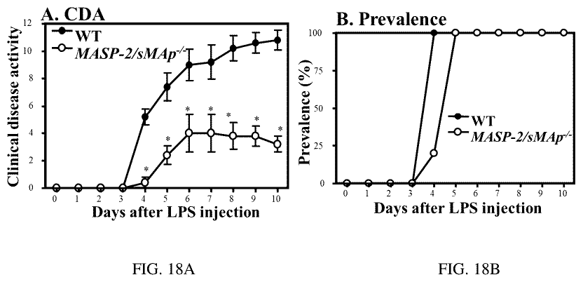

To assess the role of AdhMAp44 in another model of LP-dependent musculoskeletal inflammatory disease, we used a mouse model of Ross River virus (RRV)-induced arthritis and myositis (Morrison et al., 2006, J. Virol. 80: 737-749) (FIG. 13). Previous studies have implicated a role for the LP of the complement system in the development of RRV-induced disease, as both C3- and MBL-deficient mice exhibit reduced RRV-induced disease severity and tissue destruction compared to WT mice (Morrison et al., 2007, J. Virol. 81: 5132-5143; Morrison et al., 2006, J. Virol. 80: 737-749). To evaluate whether human MAp44 could mitigate RRV-induced disease, AdhMAp44 or AdGFP was administered in the right rear footpad to mice at days −3, 0, and 3 with inoculation with RRV in the left rear footpad at day 0. As shown, pretreatment with AdhMAp44 significantly (p<0.05) diminished the severity of RRV-induced disease signs (FIG. 13A). There was no effect on weight (FIG. 13B).

Presence of HA-Tagged Mouse MAp44 in Circulation after AdmMAp44 or AdhMAp44 Treatment

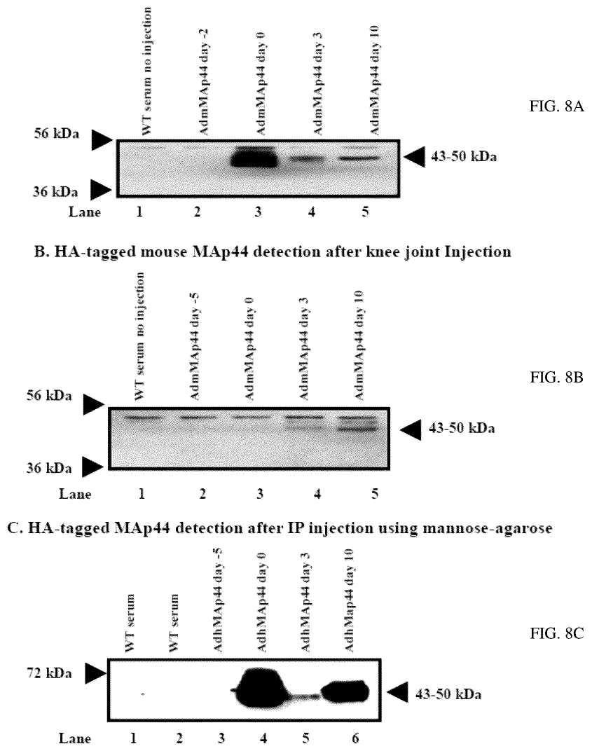

The presence of Ad-derived mouse or human MAp44 in the serum was examined, using an ELISA for the HA tag, in mice injected with AdmMAp44 or AdhMAp44 before and after the induction of CAIA (FIG. 8). Sera from mice injected i.p. with AdmMAp44 were examined at days −2, 0, 3, and 10 using Western blot analysis for the HA tag (FIG. 8A). Likewise, sera from mice injected with AdmMAp44 in the right knee joint were examined at days −5, 0, 3, and 10 using Western blot analysis for the HA tag (FIG. 8B). A band of ˜43-50 kDa was present in mice injected i.p. with AdmMAp44 but was missing at day −2 prior to injection as expected (FIG. 8A lane 2). Similarly, a band of ˜43-50 kDa was present in the sera of mice injected in the knee joint with AdmMAp44 but it was not present at day −5 prior to injection (FIG. 7B lane 2). HA was not detected using serum from untreated mice with no disease (FIG. 8A, B lane 1). The presence of HA in sera at days 0, 3 and 10 clearly indicates that mouse recombinant MAp44 was present in the circulation. In addition, human MAp44 generated after i.p. administration of AdhMAp44 was apparently functional because it bound to MBL, as confirmed using mannan-agarose beads to pull down the HA-tagged protein from serum (FIG. 8C). These results also show that AdmMAp44 or AdhMAp44 effectively transduced cells and led to recombinant protein expression in vivo regardless of the injection route. We used Western blot analysis to detect the HA tag since an ELISA to measure mouse MAp44 is not available.

### In Vivo Transduction Efficiency in the Knee Joint Synovium Through Detection of GFP and HA

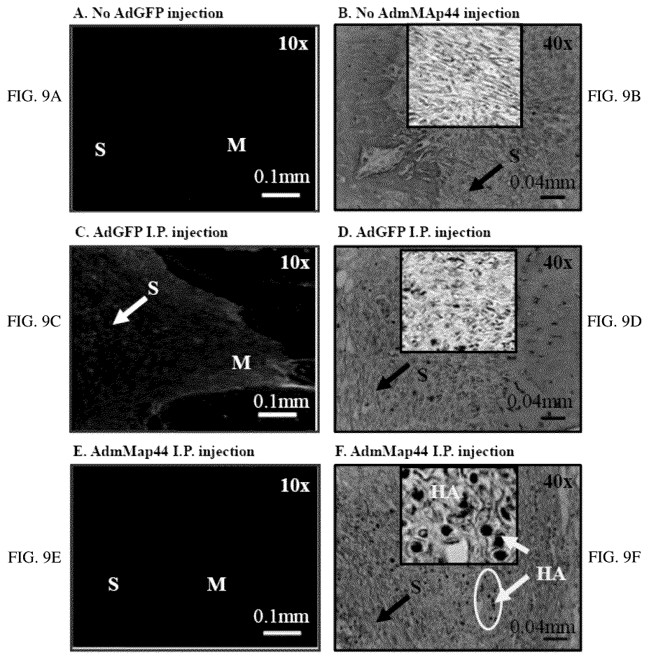

To specifically determine whether the synovium in the knee joint of mice with CAIA was transduced with AdmMAp44, we used IHS. After mice were injected i.p. with AdGFP or AdmMAp44, the presence of GFP and the HA tag was assessed in the synovium of the knee joint (FIG. 9). We found that GFP was clearly visible by IHS in the knee joints of mice with CAIA at day 10 after injection of AdGFP (FIG. 9C). In contrast, no green fluorescence was visible in mice injected with PBS (FIG. 9A) or AdmMAp44 (FIG. 9E). However, we found that the HA tag was detectable as sand grain particles in cells in mice injected i.p. with AdmMAp44 (FIG. 9F) but not in mice injected with PBS (FIG. 9B) or AdGFP (FIG. 9D). These data show that AdmMAp44 transduced a subset of cells in the synovium. We do not know the exact identity of the cells but they could be synovioctyes, lymphocytes, neutrophils and/or macrophages because of the presence in CAIA of these cell populations (Banda et al., 2012, J. Immunol. 188: 1469-1478).

Effect of AdMAp44 on Lectin, MASPs, FD and Cytokine mRNA Levels in the Knee Joints with CAIA

To determine the effect of human MAp44 expression on expression of MBL-A, MBL-C, FCN-A, MASP-1, MASP-2, MASP-3 and FD, as well as on pro-inflammatory cytokines, we measured the mRNA levels at day 10 from the knee joints of mice with CAIA injected i.p. three times with PBS, AdGFP, AdhMAp44 LD or AdhMAp44 HD (Table 2). Minimum baseline mRNA levels for all targets were present in the knee joints of mice without CAIA and without any treatment (Table 2). Liver was used as a positive control to examine the mRNA levels of all ten targets. There was a 42% and 60% decrease in the mRNA levels of FCN-A in the knee joints of mice treated with a LD or with a HD of AdhMAp44 respectively compared with AdGFP-treated mice; this decrease was significant (P<0.0017 for LD, P<0.08 for HD). Minimal levels of mRNA for MBL-A or MBL-C were present below 40 cycles of PCR in the knee joints of mice treated with PBS, AdGFP, AdhMAp44 LD, or AdhMAp44 HD. There was a statistically significant (p<0.05) decrease in the mRNA levels in the knee joints of MASP-1, MASP-2, MASP-3 and FD in mice injected with either dose of AdhMAp44 compared to mice injected with AdGFP or PBS. There was also a significant decrease (p<0.02 or greater) of 66%, 87% and 85% in the mRNA levels of TNF-α, IL-1α and IL-1β respectively in the knee joints of mice treated with either the LD or HD of AdhMAp44 (Table 2). These mRNA data show that treatment of CAIA mice with AdhMAp44 decreased the mRNA levels in the joints of FCN-A, FD, and MASPs as well as of pro-inflammatory cytokines.

TABLE 2
mRNA for factor D, MASPs and cytokines in the
knee joints of WT mice with and without CAIA¹

| mRNA | No CAIA² | PBS | AdGFP | AdhMAp44 LD³ | AdhMAp44 HD⁴ |
|---|---|---|---|---|---|
| Factor D | 28.27 ± 10.7 | 1523.6 ± 523.8 | 709.8 ± 73.9 | 295.31 ± 52.2 | 324.0 ± 120.5 |
| p | | | | 0.0017 | 0.018 |
| Decrease | | | | 58% | 54% |
| MASP-1 | 6.83 ± 0.9 | 40.6 ± 523.8 | 51.3 ± 3.7 | 31.8 ± 1.6 | 22.7 ± 3.2 |
| p | | | | 0.001 | 0.001 |
| Decrease | | | | 38% | 56% |
| MASP-3 | 1.38 ± 0.13 | 16.6 ± 2.7 | 14.2 ± 0.13 | 8.9 ± 0.76 | 5.2 ± 0.55 |
| p | | | | 0.025 | 0.0035 |
| Decrease | | | | 38% | 56% |
| MASP-2 | 0.39 ± 0.09 | 1.3 ± 0.15 | 1.2 ± 0.13 | 0.73 ± 0.1 | 0.53 ± 0.2 |
| p | | | | 0.044 | 0.010 |
| Decrease | | | | 39% | 56% |
| MBL-A⁵ | 0.29 ± 0.09 | 0.05 ± 0.01 | 0.04 ± 0.01 | 0.01 ± 0.004 | 0.01 ± 0.004 |
| MBL-C⁵ | 1.13 ± 0.27 | 0.002 ± 0.002 | 0.016 ± 0.006 | 0.006 ± 0.002 | 0.003 ± 0.002 |
| FCN-A | 145.1 ± 34.2 | 366.3 ± 55.3 | 295.6 ± 34.1 | 170.0 ± 32.6 | 117.3 ± 30.1 |
| p | | | | 0.028 | 0.069 |
| Decrease | | | | 42% | 60% |
| TNF-α | 1.06 ± 0.134 | 15.1 ± 2.1 | 12.8 ± 2.1 | 3.09 ± 0.6 | 3.9 ± 0.37 |
| p | | | | 0.002 | 0.007 |
| Decrease | | | | 76% | 66% |
| IL-1α | 1.36 ± 0.19 | 4.7 ± 1.2 | 4.0 ± 0.75 | 0.99 ± 0.23 | 0.53 ± 0.14 |
| p | | | | 0.005 | 0.005 |
| Decrease | | | | 75% | 87% |
| IL-1β | 2.91 ± 0.50 | 41.3 ± 6.1 | 29.8 ± 8.5 | 5.2 ± 0.50 | 4.40 ± 0.2 |
| p | | | | 0.02 | 0.03 |
| Decrease | | | | 83% | 85% |

¹Data are expressed in pg/ng 18s rRNA with mean ± SEM based on the indicated number of mice (n). CAIA mice treated with PBS (n = 5), AdGFP (n = 5), AdhMAp44 LD (n = 5) and AdhMAp44 HD (n = 5).
²Baseline levels for various mRNA targets from the knee joint of WT mice without CAIA and without any treatment (n = 4).
³LD = Low dose,
⁴HD = high dose.
⁵The mRNA levels of MBL-A and MBL-C were very low.
Liver from WT mice used as a positive control to measuring the mRNA levels (data not shown).
All p-values for different mRNAs in mice treated either with AdhMAp44 LD or with AdhMAp44 HD were compared with the corresponding values of WT mice treated with AdGFP.
The percent (%) decrease in mRNA levels in mice treated with AdhMAp44 in each column has been shown compared with mice treated with AdGFP.
p values were compared between mice treated either with AdGFP and AdhMAp44 LD or AdhMAp44 HD.
p < 0.05 were considered statistically significant.

### Discussion

Through the use of Ad-programmed expression of both human and mouse MAp44, these studies have revealed an unexpected essential role for the LP of the complement system in the development of CAIA. Prevention of clinical disease with adenovirus expressed MAp44 was associated with a decrease in cartilage and synovial C3 deposition, marked improvement in histologic injury scores, and decreases in local mRNA levels of LP and AP components as well pro-inflammatory cytokines. Both human and mouse MAp44 appeared to be effective in ameliorating disease, and both molecules were functional as assessed by binding to MBL in vivo. Treatment with Ad was effective using both systemic and local articular injection, with a systemic amelioration found in the latter situation likely due to the resulting effects of circulating recombinant MAp44. In sum, our studies have revealed a central role for the LP in the initiation of complement activation, and that an understanding of the molecular pathogenesis of inflammatory arthritis must expand to incorporate a role for the LP.

Prior studies using mice deficient in MBL-A/C or C4 had demonstrated no effect on the development of joint damage in CAIA. MBL is a major pattern recognition molecule within the LP, and the major means by which the LP activates C3 is through MASP-1- and MASP-2-mediated cleavage of C4 and C2 to generate the shared CP/LP C3 convertase C4b2b. The absence of an apparent effect of deletion of MBL-A/C or C4 on the evolution of tissue injury in CAIA had suggested that no major role existed for the LP. However, recent findings indicate the presence of additional pattern recognition molecules that could be important in the initiation of the LP (Kawai et al., 2002, Bioscience, biotechnology, and biochemistry 66: 2134-2145). See also, Matsushita and Fujita, 1995, Immunobiology 194: 443-448, Presanis et al., 2004, Mol. Immunol. 40: 921-929, and Degn et al., 2009, J. Immunol. 183: 7371-7378.

MAp44 interacts with MBL and ficolins with nM affinities and forms a Ca²⁺ dependent homo-dimer (Skjoedt et al., 2010, J. Biol. Chem. 285: 8234-8243). MAp44 blocks interactions between MBL and ficolins with the MASPs by competitive inhibition or displacement, disrupting the activation complexes and thus impairing LP-mediated complement activation (Degn et al., 2013, J. Immunol. 191: 1334-1345). The results from in vitro structural and functional studies have been corroborated by in vivo studies showing that MAp44 attenuates myocardial injury and arterial thrombogenesis in MBL-A/C-dependent models (Pavlov et al., 2012, Circulation 126: 2227-2235). Therefore, MAp44 was suggested as a natural in vivo endogenous inhibitor of the LP (Pavlov et al., 2012, Circulation 126: 2227-2235).

Although the CAIA model does not require MBL-A/C engagement, we hypothesized that if MAp44 inhibits other collectin-MASP interactions, it could affect the development of CAIA in mice. Since large amounts of purified MAp44 protein were not available for experiments, we used AdhMAp44 and AdmMAp44 to generate sustained in vivo production of human and mouse HA-tagged MAp44 for CAIA studies. We found that there was more 40% decrease in C3 activation via LP when mice were treated with AdhMAp44 vs AdGFP. The concentration of MAp44 in human serum is 1.7 μg/ml (Degn et al., 2010, J. Immunol. Methods 361: 37-50; Skjoedt et al., 2010, J. Biol. Chem. 285: 8234-8243). Although the levels of mouse MAp44 are not known, the demonstration of a marked clinical effect showed that we had achieved a therapeutically effective dose. This is due to the fact that there was a huge 1000-fold increase in the levels of MAp44, at day 0, just with a single injection of AdhMAp44 at day −5 and these data were consistent with the western blot analysis of MAp44. The measured levels of human MAp44 were in the 100-300 ng/ml range using serum obtained at 10 days, which was a point of lower levels as assessed by pull-down experiments.

Adenovirus type 5 was chosen as a delivery vehicle for the current studies because it uses the Coxsackie-Adenovirus Receptor (CAR, a cell adhesion molecule) to bind to cells (Bakker et al., 2001, Gene Ther. 8: 1785-1793), and subsequent internalization takes place by binding of an Arg-Gly-Asp (RGD) sequence to the integrins αvβ5 and αvβ3 (Bai et al., 1993, J. Virol. 67: 5198-5205; Mathias et al., 1994, J. Virol. 68: 6811-6814; Wickham et al., 1993, Cell 73: 309-319). Synoviocytes express CAR or RGD binding integrin on their surface, which are used by adenoviruses to enter into cells. We found by using FACS analysis that a fibroblast like synoviocyte (FLS) cell line driven from the synovium of a mouse with CIA expressed a substantial levels (˜60%) of the integrin, αvβ5 on their surface (data not shown). IHS data also showed that RGD binding integrins are highly expressed in the mouse synovium with and without arthritis (data not shown) (Nikkari et al., 1995, J. Rheumatol. 22: 16-23; Pirila and Heino, 1996, J. Rheumatol. 23: 1691-1698; Rinaldi et al., 1997, Ann. Rheum. Dis. 56: 729-736) and the inclusion of the RGD sequence in the construct markedly improves the delivery into the synovial cells (Heja et al., 2012, Proc. Natl. Acad. Sci. USA 109: 10498-10503) because administration of AdmMAp44 without RGD although partially attenuated the CAIA but it was not highly statistically significant (data not shown). Therefore RGD sequences in AdMAp44 served as an effective delivery vehicle to homing AdMAp44 the synovium in the knee joints of mice without affecting the disease itself because AdGFP also contained RGD. For example, adenovirus containing the mouse interleukin 1 receptor antagonist (mIL-1Ra) inhibited collagen-induced arthritis (CIA) (Bakker et al., 2001, Gene Ther. 8: 1785-1793), and it has been shown that Ad vectors carrying the human adiponectin APN (Ad-APN) gene significantly reduced CIA and C3 deposition in the knee joints (Ebina et al., 2009, Biochem. Biophys. Res. Commun. 378: 186-191).

CAIA is an appropriate model in which to study the role of the LP. The complement system, a part of innate immunity, protects from invading pathogens but also plays a central role in the pathological process of an autoimmune and inflammatory disease such as RA. Our studies have previously shown that in CAIA, the AP is the main contributor to the development of tissue injury (Banda et al., 2006, J. Immunol. 177: 1904-1912). Additionally, MASP-1 and MASP-3 cleave a zymogen of FD called pro-FD (Takahashi et al., 2010, J. Exp. Med. 207: 29-37). It was shown that mice lacking MASP-1 and MASP-3 lack both the LP and have reduced AP activity (Takahashi et al., 2010, J. Exp. Med. 207: 29-37; Takahashi et al., 2008, J. Immunol. 180: 6132-6138). Similarly, it has been reported that patients deficient in MASP-1 and MASP-3 have reduced but detectable AP activity (Degn et al., 2012, J. Immunol. 189: 3957-3969). No cleavage of Pro-FD was observed in the circulation of fH−/−/MASP-1/3−/− mice; AP activity was reduced in these mice and activation was possible only after injecting Cobra Venom Factor (Ruseva et al., 2013, Clin. Exp. Immunol.). Overall, MASP-1/3−/− mice exhibit defective AP activation because there is only pro-FD and not mature FD in circulation, even in the presence of plasmin, thrombin and kallikreins (Takahashi et al., 2010, J. Exp. Med. 207: 29-37; Takahashi et al., 2008, J. Immunol. 180: 6132-6138; Banda et al., 2010, J. Immunol. 185: 5598-5606). Consistent with these observations, we have shown that MASP-1/3−/− mice not only have defective AP of the complement system but are markedly resistant to CAIA (Banda et al., 2010, J. Immunol. 185: 5598-5606). Thus, for the development of CAIA, the AP of the complement system is necessary and mice lacking any component of the AP are resistant to arthritis (Banda et al., 2006, J. Immunol. 177: 1904-1912; Kemper et al., 2010, Annu. Rev. Immunol. 28: 131-155; Banda et al., 2010, Clin. Exp. Immunol. 159: 100-108).

In the current CAIA studies we have made several new observations. First, AdhMAp44 dramatically attenuates CAIA in mice. Recombinant hMAp44 was detectable on day 0, day 3 and on day 10 in the circulation of CAIA mice treated with AdhMAp44. In addition, administration of AdhMAp44 diminished the severity of RRV-induced arthritis in mice. Second, AdmMAp44 also attenuates CAIA in mice using either a systemic or local injection as the delivery route. Third, there was a decrease in the levels of C5a in the circulation of WT mice treated with AdhMAp44 compared with AdGFP-treated mice, which is consistent with the intended effect of MAp44 expression (FIG. 3A). Fourth, MAp44 not only inhibited interactions between MASPs and its ligands but also resulted in reduced levels of MASP-1, MASP-2, MASP-3 and pro-FD mRNA expression, as well as pro-inflammatory cytokine expression, in the knee joints of mice treated with AdhMAp44. And fifth, the ameliorative effects of MAp44 are not specific to one model of arthritis, as when we used another mouse model, RRV-induced arthritis that is not dependent on the AP of complement but it is partially dependent on the LP ligand MBL (Morrison et al., 2007, J. Virol. 81: 5132-5143; Morrison et al., 2008, J. Virol. 82: 11263-11272; Morrison et al., 2006, J. Virol. 80: 737-749), we again found that AdhMAp44 significantly decreased arthritis.

Finally, we conclude that Ad-mediated gene transfer of MAp44 can be used as a potential tool for the treatment of arthritis. Human MAp44 was present in the circulation, and MAp44 delivered by this approach can have long-term inhibitory effects on inflammatory arthritis even with reduced transduction efficiency. Furthermore, an Ad gene delivery system is highly efficient at transferring genes to a variety of proliferating and quiescent cells both in vitro and in vivo (Wilson, 1996, N. Engl. J. Med. 334: 1185-1187). It has also been shown that genetically modified Ad5 vectors with short-shafted fibers are highly efficient in transduction of RA fibroblast-like synoviocytes (FLS) and human and murine synovium (Toh et al., 2005, J. Immunol. 175: 7687-7698). The marked prevention of disease using AdhMAp44 sequences provides evidence that Ad vectors containing complement inhibitor genes and/or use of recombinant MAp44 alone should be evaluated as a treatment for inflammatory arthritis in humans.

### Example 2: Mouse Models of Arthritis for MAp44 Studies

The following strains of mice are utilized for rheumatoid arthritis (RA) studies: C57BL/6 (Jackson Laboratories) and DBA/1 lacJ (Jackson Laboratories). C57BL/6 and DBA/1 lacJ mice are susceptible to anti-collagen antibody (passive) and collagen-induced arthritis (active), respectively, and are widely used to study inflammatory arthritis. Both male and female mice are equally susceptible to developing arthritis following immunization with bovine type II collagen or injection with a cocktail of monoclonal antibodies to collagen (Arthrogen).

We use two mouse models of RA called collagen antibody-induced arthritis (CAIA) and collagen-induced arthritis (CIA). CAIA is a mouse model of RA dependent on the complement system and CIA is dependent on T-cells, B-cells and complement. Eight to ten week old C57BL/6 and DBA1/lacJ and various KO males and females are anesthetized with 500 μl of avertin (intraperitoneal injection: dose 0.75 mg/g). Avertin is an anesthetic agent used for all of our surgical procedures. It is available from Sigma.

For CAIA, mice are injected intraperitonealy (IP) with a cocktail of monoclonal anti-collagen antibodies (Arthrogen cocktail contains five antibodies). C57BL/6 mice require 8 mg/mouse of Arthrogen. At day 3, all mice are injected with LPS (Lipoplysaccride) using a 25 G needle intraperitonealy (50 μl total volume at a concentration of 50 μg/mouse). The injection of LPS is necessary to cycle disease in this model. Mice injected with anti-CII antibodies alone or LPS develop very low levels of arthritis. In contrast, mice injected with anti-CII antibodies followed by an injection of LPS develop severe arthritis (FIG. 15).

Mice start showing the signs of clinical disease at day 4 and all mice are sacrificed at day 10. Mice start showing the signs of clinical disease at day 4 immediately after LPS injection as mentioned earlier. At day 4 both fore paws and hind paws become slightly red. Later on the knee joint swells due to ankylosis. We have already established and published the following criteria to examine the clinical disease in mice.

For CIA, mice at day 0 are injected intradermally (total volume 100 ul) at the base of the tail using a 25 G needle with 200 μg of bovine collagen type II in Incomplete Freunds Adjuvant containing 4 mg/ml of inactivated Mycobacterium tuberculosis. After three weeks (21 days), the mice are anesthetized with avertin and receive a second booster injection of 200 μg of bovine collagen type II in Incomplete Freund's Adjuvant containing 4 mg/ml of inactivated Mycobacterium. The booster injection at day 21 is necessary to cycle the disease and for the production of antibodies necessary for the development of arthritis. Both injections of collagen are given intradermally. This protocol is widely used to induce collagen-induced arthritis in DBA1/J mice. Freund's adjuvant and inactivated Mycobacterium tuberculosis are necessary to induce this model of arthritis. Mice do not develop consistent and severe levels of arthritis if we do not use inactivated M. tuberculosis along with IFA. To examine the effect of different proteins and antibodies mice must develop severe arthritis, otherwise we cannot compare outcomes for different treatment groups. Animals are monitored daily for the development of arthritis. Arthritis usually occurs 4 to 5 weeks following the first collagen injection. The early signs of arthritis are the appearance of redness on the fore and hind limbs. The control group consists of WT mice. At 2-3 weeks after the onset of arthritis, all groups of animals are sacrificed by giving anesthesia to obtain blood followed by cervical dislocation. For IV or knee joint injections we use a 1 cc U-100 27G5/8 (0.40×1600) insulin syringe. This syringe has a permanently attached needle.

Clinical disease activity (CDA) is scored on a 3-point scale per paw: 0=normal joint; 1=slight inflammation and redness; 2=severe erythema and swelling affecting the entire paw with inhibition of use; and 3=deformed paw or joint with ankylosis, joint rigidity, and loss of function. The total score for clinical disease activity is based on all 4 paws and is a maximum of 12 for each mouse.

At day 10 for CAIA or at day 35 for CIA studies, both forepaws and the entire right hind limb, including the paw, ankle and knee, are surgically removed from all mice and fixed immediately in 10% buffered formalin (Biochemical Sciences, Inc., Swedesboro, NJ). The preparation of tissue samples and histological analysis are performed as described previously (Banda et al., 2006, J. Immunol. 177: 1904-1912; Banda et al., 2007, J. Immunol. 179: 4101-4109). All sections are read by a trained observer who is also blinded to the mouse types and to the clinical disease activity score of each mouse. The joint sections are scored for changes in inflammation, pannus, cartilage damage, and bone damage, from a scale of 0-5, and the overall score is calculated as the total of the four individual parameters. Each parameter is represented as the mean value for 5 joints per mouse.

At sacrifice on day 10 or at day 35, the entire right hind limb, including the paw, ankle and knee, is surgically removed from all mice and fixed immediately in 10% buffered formalin (Biochemical Sciences, Inc., Swedesboro, NJ). C3 deposition in the joints of all experimental mice is assessed immunohistochemically by using polyclonal goat anti-mouse C3 antisera (ICN Pharmaceuticals, Aurora, OH). Histology sections are incubated with anti-murine C3 antibodies overnight at 4° C. (Banda et al., 2006, J. Immunol. 177: 1904-1912; Banda et al., 2007, J. Immunol. 179: 4101-4109). The tissue sections are sequentially incubated with biotinylated rabbit anti-goat immunoglobulin (Vector Laboratories, Burlington, CA), and then additionally treated with Dako LSAB2 Steptavidin-HRP (DakoCytomation, Carpinteria, CA). Staining is developed with Liquid DAB+(DakoCytomation, Carpentaria, CA) and counterstained with Hematoxylin. Immunohistochemical staining for MAC is done using identical methods.

C3 immunohistochemical stain scoring is performed as follows: both the synovium and surrounding tissue is scored based upon a 3 point scoring system, in which 0 represented no staining and 3 represents 3+ staining. The staining in the cartilage is also assessed. The criteria for cartilage staining is as follows: 0—no staining present, 0.5—one area of minimal staining of chondrocytes in one joint, 1—one area of moderate staining of chondrocytes in one joint, 2—multiple areas of moderate staining of chondrocytes—multiple joints affected, 3—multiple areas of intense staining of chondryocytes and/or diffuse multi-focal staining of articular cartilage lesions. For each animal, the synovium and cartilage scores are determined separately for each of the 5 joints. A sum total animal score (all 5 joints, with a maximum score of 30) and a five joint mean animal score (maximum of 6) is determined, as well as sums (maximum of 15) and means (maximum of 3) for each of the individual (synovium or cartilage) parameters. We have already published the above mentioned immunohistochemical scoring methods for C3 and IgG (Banda et al., 2006, J. Immunol. 177: 1904-1912; Banda et al., 2007, J. Immunol. 179: 4101-4109; Banda et al., 2012, J. Immunol. 188: 1469-1478).

SEQUENCES
B4 CDR-L1 amino acid sequence
(SEQ ID NO: 1)
SSISSNY
B4 CDR-L2 amino acid sequence
(SEQ ID NO: 2)
RTS
B4 CDR-L3 amino acid sequence
(SEQ ID NO: 3)
QQGSSIPRTRSEGAPSWK
B4 CDR-H1 amino acid sequence
(SEQ ID NO: 4)
GYTFTSYW
B4 CDR-H2 amino acid sequence
(SEQ ID NO: 5)
IGPNSGGT
B4 CDR-H3 amino acid sequence
(SEQ ID NO: 6)
ARRMVKGCYGLLGPRDHGHRLL
B4 CDR-L1 amino acid sequence
(SEQ ID NO: 7)
QSIVHSNGNTY
B4 CDR-L2 amino acid sequence
(SEQ ID NO: 8)
KVS
B4 CDR-L3 amino acid sequence
(SEQ ID NO: 9)
FQGSHVPYT
B4 CDR-H1 amino acid sequence
(SEQ ID NO: 10)
GYTFTDYY
B4 CDR-H2 amino acid sequence
(SEQ ID NO: 11)
INPNNGGT
B4 CDR-H3 amino acid sequence
(SEQ ID NO: 12)
ARYDYAWYFDV
B4 VL amino acid sequence
(SEQ ID NO: 13)
DIELTQSPTTMAASPGEKITITCSASSSISSNYLHWYQQKPGFSP
KLLIYRTSNLASGVPARFSGSGSGTSYSLTIGTMEAEDVATYYCQ
QGSSIPRTRSEGAPSWK
B4 VL amino acid sequence
(SEQ ID NO: 14)
DVLMTQTPLSLPVSLGDQASISCRSSQSIVHSNGNTYLEWYLQKP
GQSPKLLIYKVSNRFSGVPDRFSGSGSGTDFTLKISRVEAEDLGV
YYCFQGSHVPYTFGGGTKLEIK
B4 VH amino acid sequence
(SEQ ID NO: 15)
VKLQESGAELVKPGASVKLSCKASGYTFTSYWMHWVKQRPGRGLE
WIGRIGPNSGGTKYNEKFKSKATLTVDKPSSTAYMQLSSLTSEDS
AVYYCARRMVKGCYGLLGPRDHGHRLL
B4 VH amino acid sequence
(SEQ ID NO: 16)
VKLQESGPELVKPGASVKISCKASGYTFTDYYMNWVKQSHGKSLE
WIGDINPNNGGTSYNQKFKGKATLTVDKSSSTAYMELRSLTSEDS
AVYYCARYDYAWYFDVWGQGTTVTVSS
B4 scFV amino acid sequence
(SEQ ID NO: 17)
HHHHHHVKLQESGAELVKPGASVKLSCKASGYTFTSYWMHWVKQR
PGRGLEWIGRIGPNSGGTKYNEKFKSKATLTVDKPSSTAYMQLSS
LTSEDSAVYYCARRMVKGCYGLLGPRDHGHRLLKGRIPAHWRPLL
VDPSSVPSLASGGGGGSGGGGSWISAEFALDIELTQSPTTMAASP
GEKITITCSASSSISSNYLHWYQQKPGFSPKLLIYRTSNLASGVP
ARFSGSGSGTSYSLTIGTMEAEDVATYYCQQGSSIPRTRSEGAPS
WK
B4 scFV amino acid sequence
(SEQ ID NO: 18)
MSVPTQVLGLLLLWLTDARCVKLQESGPELVKPGASVKISCKASG
YTFTDYYMNWVKQSHGKSLEWIGDINPNNGGTSYNQKFKGKATLT
VDKSSSTAYMELRSLTSEDSAVYYCARYDYAWYFDVWGQGTTVTV
SSGGGGSGGGGSGGGGDVLMTQTPLSLPVSLGDQASISCRSSQSI
VHSNGNTYLEWYLQKPGQSPKLLIYKVSNRFSGVPDRESGSGSGT
DFTLKISRVEAEDLGVYYCFQGSHVPYTFGGGTKLEIKRIEGRHH
HHHH
B4 VL nucleic acid sequence
(SEQ ID NO: 19)
GACATTGAGCTCACCCAGTCTCCAACCACCATGGCTGCATCTCCC
GGGGAGAAGATCACTATCACCTGCAGTGCCAGCTCAAGTATAAGT
TCCAATTACTTGCATTGGTATCAGCAGAAGCCAGGATTCTCCCCT
AAACTCTTGATTTATAGGACATCCAATCTGGCTTCTGGAGTCCCA
GCTCGCTTCAGTGGCAGTGGGTCTGGGACCTCTTACTCTCTCACA
ATTGGCACCATGGAGGCTGAAGATGTTGCCACTTACTACTGCCAG
CAGGGTAGTAGTATACCACGTACACGTTCGGAGGGGGCACCAAGC
TGGAAA
B4 VL nucleic acid sequence
(SEQ ID NO: 20)
GATGTTTTGATGACCCAAACTCCACTCTCCCTGCCTGTCAGTCTT
GGAGATCAAGCCTCCATCTCTTGCAGATCTAGTCAGAGCATTGTA
CATAGTAATGGAAACACCTATTTAGAATGGTACCTGCAGAAACCA
GGCCAGTCTCCAAAGCTCCTGATCTACAAAGTTTCCAACCGATTT
TCTGGGGTCCCAGACAGGTTCAGTGGCAGTGGATCAGGGACAGAT
TTCACACTCAAGATCAGCAGAGTGGAGGCTGAGGATCTGGGAGTT
TATTACTGCTTTCAAGGTTCACATGTTCCGTACACGTTCGGAGGG
GGGACCAAGCTGGAAATAAAACG
B4 VH nucleic acid sequence
(SEQ ID NO: 21)
GTGAAACTGCAGGAGTCAGGGGCTGAGCTTGTGAAGCCTGGGGCT
TCAGTGAAGCTGTCCTGCAAGGCTTCTGGCTACACCTTCACCAGC
TACTGGATGCACTGGGTGAAGCAGAGGCCTGGACGAGGCCTTGAG
TGGATTGGAAGGATTGGTCCTAATAGTGGTGGTACTAAGTACAAT
GAGAAGTTCAAGAGCAAGGCCACACTGACTGTAGACAAACCCTCC
AGCACAGCCTACATGCAGCTCAGCAGCCTGACATCTGAGGACTCT
GCGGTCTATTATTGTGCAAGAAGAATGGTAAAGGGGTGCTATGGA
CTACTGGGGCCAAGGGACCACGGTCACCGTCTCCTCA
B4 VH nucleic acid sequence
(SEQ ID NO: 22)
GTGAAGCTGCAGGAGTCTGGACCTGAGCTGGTGAAGCCTGGGGCT
TCAGTGAAGATATCCTGTAAGGCTTCTGGATACACGTTCACTGAC
TACTACATGAACTGGGTGAAGCAGAGCCATGGAAAGAGCCTTGAG
TGGATTGGAGATATTAATCCTAACAATGGTGGTACTAGCTACAAC
CAGAAGTTCAAGGGCAAGGCCACATTGACTGTAGACAAGTCCTCC
AGCACAGCCTACATGGAGCTCCGCAGCCTGACATCTGAGGACTCT
GCAGTCTATTACTGTGCAAGATATGATTACGCTTGGTACTTCGAT
GTCTGGGGCCAAGGGACCACGGTCACCGTCTCCTCA
B4 scFV nucleic acid sequence
(SEQ ID NO: 23)
GCCGCCACCATGAGTGTGCCCACTCAGGTCCTGGGGTTGCTGCTG
CTGTGGCTTACAGATGCCAGATGTGTGAAGCTGCAGGAGTCTGGA
CCTGAGCTGGTGAAGCCTGGGGCTTCAGTGAAGATATCCTGTAAG
GCTTCTGGATACACGTTCACTGACTACTACATGAACTGGGTGAAG
CAGAGCCATGGAAAGAGCCTTGAGTGGATTGGAGATATTAATCCT
AACAATGGTGGTACTAGCTACAACCAGAAGTTCAAGGGCAAGGCC
ACATTGACTGTAGACAAGTCCTCCAGCACAGCCTACATGGAGCTC
CGCAGCCTGACATCTGAGGACTCTGCAGTCTATTACTGTGCAAGA
TATGATTACGCTTGGTACTTCGATGTCTGGGGCCAAGGGACCACG
GTCACCGTCTCCTCAGGCGGAGGTGGGTCGGGTGGCGGCGGATCT
GGCGGAGGTGGGGATGTTTTGATGACCCAAACTCCACTCTCCCTG
CCTGTCAGTCTTGGAGATCAAGCCTCCATCTCTTGCAGATCTAGT
CAGAGCATTGTACATAGTAATGGAAACACCTATTTAGAATGGTAC
CTGCAGAAACCAGGCCAGTCTCCAAAGCTCCTGATCTACAAAGTT
TCCAACCGATTTTCTGGGGTCCCAGACAGGTTCAGTGGCAGTGGA
TCAGGGACAGATTTCACACTCAAGATCAGCAGAGTGGAGGCTGAG
GATCTGGGAGTTTATTACTGCTTTCAAGGTTCACATGTTCCGTAC
ACGTTCGGAGGGGGGACCAAGCTGGAAATAAAACGGATCGAAGGC
CGGCATCACCATCATCACCACTGATAG
CHO optimized B4 scFV nucleic acid sequence
(SEQ ID NO: 24)
ATGTCCGTGCCTACCCAGGTGCTCGGACTCCTGCTGCTGTGGCTC
ACCGACGCCAGGTGTGTGAAGCTGCAGGAGAGCGGACCCGAGCTG
GTGAAGCCTGGAGCCTCCGTGAAGATCAGCTGCAAGGCTTCCGGA
TACACCTTCACCGACTACTATATGAACTGGGTGAAGCAGAGCCAC
GGCAAGAGCCTGGAGTGGATCGGCGACATCAACCCTAACAACGGC
GGCACCTCCTACAACCAGAAGTTCAAGGGCAAGGCTACACTGACC
GTGGACAAGTCCTCCAGCACCGCCTACATGGAGCTCAGGAGCCTG
ACCTCCGAGGATTCCGCCGTCTATTACTGTGCCCGGTACGACTAC
GCCTGGTATTTCGACGTGTGGGGCCAGGGCACAACCGTCACAGTC
TCCAGCGGAGGAGGAGGAAGCGGCGGCGGAGGATCCGGAGGCGGA
GGCGATGTCCTGATGACACAGACACCTCTGAGCCTCCCCGTGAGC
CTGGGAGACCAAGCCTCCATCTCCTGCAGGTCCTCCCAGTCCATC
GTGCACAGCAATGGCAACACCTACCTGGAGTGGTATCTGCAGAAG
CCTGGCCAGTCCCCCAAGCTGCTGATCTACAAGGTGTCCAACCGG
TTCAGCGGCGTCCCTGACAGGTTCTCCGGATCCGGAAGCGGCACA
GATTTCACCCTGAAGATCAGCAGGGTCGAGGCCGAGGACCTGGGA
GTGTACTACTGCTTCCAGGGCTCCCATGTCCCTTACACCTTCGGC
GGCGGCACCAAACTGGAGATCAAGCGGATCGAGGGCAGGCATCAC
CACCATCACCACTGA
C2 CDR-L1 amino acid sequence
(SEQ ID NO: 25)
KSVSTSGYSY
C2 CDR-L2 amino acid sequence
(SEQ ID NO: 26)
LVS
C2 CDR-L3 amino acid sequence
(SEQ ID NO: 27)
QHIRELTRSEGGPSWK
C2 CDR-H1 amino acid sequence
(SEQ ID NO: 28)
GYTFTSYW
C2 CDR-H2 amino acid sequence
(SEQ ID NO: 29)
INPSNGGT
C2 CDR-H3 amino acid sequence
(SEQ ID NO: 30)
ARRGIRLRHFDY
C2 CDR-L1 amino acid sequence
(SEQ ID NO: 31)
QDVGTA
C2 CDR-L2 amino acid sequence
(SEQ ID NO: 32)
WAS
C2 CDR-L3 amino acid sequence
(SEQ ID NO: 33)
QQYSSYPLT
C2 VL amino acid sequence
(SEQ ID NO: 34)
DIVMTQSPASLAVSLGQRATISYRASKSVSTSGYSYMHWNQQKPG
QPPRLLIYLVSNLESGVPARFSGSGSGTDFTLNIHPVEEEDAATY
YCQHIRELTRSEGGPSWK
C2 VL amino acid sequence
(SEQ ID NO: 35)
DIQMTQSPKFMSTSVGDRVSITCKASQDVGTAVAWYQQKPGQSPK
LLIYWASTRHTGVPDRFTGSGSGTDFTLTISNVQSEDLADYFCQQ
YSSYPLTFGAGTKLELK
C2 VH amino acid sequence
(SEQ ID NO: 36)
VKLQESGTELVKPGASVKLSCKASGYTFTSYWMHWVKQRPGQGLE
WIGNINPSNGGTNYNEKFKSKATLTVDKSSSTAYMQLSSLTSEDS
AVYYCARRGIRLRHFDYWGQGTTVTVS
C2 scFV amino acid sequence
(SEQ ID NO: 37)
VKLQESGTELVKPGASVKLSCKASGYTFTSYWMHWVKQRPGQGLE
WIGNINPSNGGTNYNEKFKSKATLTVDKSSSTAYMQLSSLTSEDS
AVYYCARRGIRLRHFDYWGQGTTVTVSSRANSADIHHTGGRSSMH
LEGPIRPIVSRISGGGGGSGGGGSWISAEFALDIVMTQSPASLAV
SLGQRATISYRASKSVSTSGYSYMHWNQQKPGQPPRLLIYLVSNL
ESGVPARFSGSGSGTDFTLNIHPVEEEDAATYYCQHIRELTRSEG
GPSWK
C2 scFV amino acid sequence
(SEQ ID NO: 38)
MSVPTQVLGLLLLWLTDARCVKLQESGTELVKPGASVKLSCKASG
YTFTSYWMHWVKQRPGQGLEWIGNINPSNGGTNYNEKFKSKATLT
VDKSSSTAYMQLSSLTSEDSAVYYCARRGIRLRHFDYWGQGTTVT
VSSGGGGSGGGGSGGGGSDIQMTQSPKFMSTSVGDRVSITCKASQ
DVGTAVAWYQQKPGQSPKLLIYWASTRHTGVPDRFTGSGSGTDFT
LTISNVQSEDLADYFCQQYSSYPLTFGAGTKLELKRIEGRHHHHH
H
C2 VL nucleic acid sequence
(SEQ ID NO: 39)
GACATTGTGATGACACAGTCTCCTGCTTCCTTAGCTGTATCTCTG
GGGCAGAGGGCCACCATCTCATACAGGGCCAGCAAAAGTGTCAGT
ACATCTGGCTATAGTTATATGCACTGGAACCAACAGAAACCAGGA
CAGCCACCCAGACTCCTCATCTATCTTGTATCCAACCTAGAATCT
GGGGTCCCTGCCAGGTTCAGTGGCAGTGGGTCTGGGACAGACTTC
ACCCTCAACATCCATCCTGTGGAGGAGGAGGATGCTGCAACCTAT
TACTGTCAGCACATTAGGGAGCTTACACGTTCGGAGGGGGGACCA
AGCTGGAAA
C2 VL nucleic acid sequence
(SEQ ID NO: 40)
GACATCCAGATGACCCAGTCTCCCAAATTCATGTCCACATCAGTA
GGAGACAGGGTCAGCATCACCTGCAAGGCCAGTCAGGATGTGGGT
ACTGCTGTAGCCTGGTATCAACAGAAACCAGGGCAATCTCCTAAA
CTACTGATTTACTGGGCATCCACCCGGCACACTGGAGTCCCTGAT
CGCTTCACAGGCAGTGGATCTGGGACAGATTTCACTCTCACCATT
AGCAATGTGCAGTCTGAAGACTTGGCAGATTATTTCTGTCAGCAA
TATAGCAGCTATCCTCTCACGTTCGGTGCTGGGACCAAGCTGGAG
CTGAAAC
C2 VH nucleic acid sequence
(SEQ ID NO: 41)
GTGAAACTGCAGGAGTCTGGGACTGAACTGGTGAAGCCTGGGGCT
TCAGTGAAGCTGTCCTGCAAGGCTTCTGGCTACACCTTCACCAGC
TACTGGATGCACTGGGTGAAGCAGAGGCCTGGACAAGGCCTTGAG
TGGATTGGAAATATTAATCCTAGCAATGGTGGTACTAACTACAAT
GAGAAGTTCAAGAGCAAGGCCACACTGACTGTAGACAAATCCTCC
AGCACAGCCTACATGCAGCTCAGCAGCCTGACATCTGAGGACTCT
GCGGTCTATTATTGTGCAAGAAGAGGCATACGGTTACGACACTTT
GACTACTGGGGCCAAGGGACCACGGTCACCGTCTCC
C2 scFV nucleic acid sequence
(SEQ ID NO: 42)
GCCGCCACCATGAGTGTGCCCACTCAGGTCCTGGGGTTGCTGCTG
CTGTGGCTTACAGATGCCAGATGTGTGAAACTGCAGGAGTCTGGG
ACTGAACTGGTGAAGCCTGGGGCTTCAGTGAAGCTGTCCTGCAAG
GCTTCTGGCTACACCTTCACCAGCTACTGGATGCACTGGGTGAAG
CAGAGGCCTGGACAAGGCCTTGAGTGGATTGGAAATATTAATCCT
AGCAATGGTGGTACTAACTACAATGAGAAGTTCAAGAGCAAGGCC
ACACTGACTGTAGACAAATCCTCCAGCACAGCCTACATGCAGCTC
AGCAGCCTGACATCTGAGGACTCTGCGGTCTATTATTGTGCAAGA
AGAGGCATACGGTTACGACACTTTGACTACTGGGGCCAAGGGACC
ACGGTCACCGTCTCCTCTGGCGGAGGTGGGTCGGGTGGCGGCGGA
TCTGGCGGAGGTGGGTCGGACATCCAGATGACCCAGTCTCCCAAA
TTCATGTCCACATCAGTAGGAGACAGGGTCAGCATCACCTGCAAG
GCCAGTCAGGATGTGGGTACTGCTGTAGCCTGGTATCAACAGAAA
CCAGGGCAATCTCCTAAACTACTGATTTACTGGGCATCCACCCGG
CACACTGGAGTCCCTGATCGCTTCACAGGCAGTGGATCTGGGACA
GATTTCACTCTCACCATTAGCAATGTGCAGTCTGAAGACTTGGCA
GATTATTTCTGTCAGCAATATAGCAGCTATCCTCTCACGTTCGGT
GCTGGGACCAAGCTGGAGCTGAAACGGATCGAAGGCCGGCATCAC
CATCATCACCACTGATAG
CHO optimized C2 scFV nucleic acid sequence
(SEQ ID NO: 43)
ATGAGCGTGCCTACACAGGTGCTCGGCCTGCTGCTCCTCTGGCTG
ACAGACGCCCGGTGTGTGAAGCTGCAGGAGTCCGGAACCGAGCTG
GTGAAACCTGGCGCCAGCGTGAAACTGAGCTGCAAAGCCAGCGGA
TACACCTTCACCTCCTACTGGATGCACTGGGTGAAACAGAGGCCT
GGCCAGGGCCTGGAATGGATTGGCAACATCAACCCCAGCAACGGC
GGCACCAACTACAATGAGAAGTTCAAGAGCAAGGCCACCCTGACC
GTGGATAAGTCCTCCTCCACCGCCTACATGCAGCTGTCCTCCCTC
ACCTCCGAGGACAGCGCCGTCTATTACTGTGCCAGGCGGGGCATC
AGGCTGAGGCACTTCGACTACTGGGGCCAAGGCACAACCGTCACC
GTGAGCTCCGGAGGAGGAGGCAGCGGAGGCGGAGGCTCCGGCGGA
GGCGGAAGCGACATTCAGATGACCCAGAGCCCCAAGTTCATGTCC
ACCTCCGTCGGCGACAGGGTGAGCATCACCTGTAAGGCCAGCCAG
GATGTCGGCACAGCTGTGGCCTGGTACCAGCAGAAGCCCGGCCAG
TCCCCCAAGCTGCTGATCTACTGGGCTTCCACAAGGCATACCGGC
GTCCCCGATAGGTTCACAGGCTCCGGCTCCGGCACCGACTTCACA
CTCACCATCAGCAACGTCCAGTCCGAGGACCTGGCCGACTACTTC
TGCCAGCAGTACTCCAGCTACCCCCTCACCTTCGGCGCTGGCACC
AAGCTGGAACTCAAGCGGATCGAGGGCAGGCATCACCACCATCAC
CACTGATAG
MAp44 amino acid sequence
(SEQ ID NO: 44)
MRWLLLYYALCFSLSKASAHTVELNNMFGQIQSPGYPDSYPSDSE
VTWNITVPDGFRIKLYFMHFNLESSYLCEYDYVKVETEDQVLATF
CGRETTDTEQTPGQEVVLSPGSFMSITFRSDFSNEERFTGFDAHY
MAVDVDECKEREDEELSCDHYCHNYIGGYYCSCRFGYILHTDNRT
CRVECSDNLFTQRTGVITSPDFPNPYPKSSECLYTIELEEGFMVN
LQFEDIFDIEDHPEVPCPYDYIKIKVGPKVLGPFCGEKAPEPIST
QSHSVLILFHSDNSGENRGWRLSYRAAGNECPELQPPVHGKIEPS
QAKYFFKDQVLVSCDTGYKVLKDNVEMDTFQIECLKDGTWSNKIP
TCKKNEIDLESELKSEQVTE
MAp44 nucleic acid sequence
(SEQ ID NO: 45)
ATGAGGTGGCTGCTTCTCTATTATGCTCTGTGCTTCTCCCTGTCA
AAGGCTTCAGCCCACACCGTGGAGCTAAACAATATGTTTGGCCAG
ATCCAGTCGCCTGGTTATCCAGACTCCTATCCCAGTGATTCAGAG
GTGACTTGGAATATCACTGTCCCAGATGGGTTTCGGATCAAGCTT
TACTTCATGCACTTCAACTTGGAATCCTCCTACCTTTGTGAATAT
GACTATGTGAAGGTAGAAACTGAGGACCAGGTGCTGGCAACCTTC
TGTGGCAGGGAGACCACAGACACAGAGCAGACTCCCGGCCAGGAG
GTGGTCCTCTCCCCTGGCTCCTTCATGTCCATCACTTTCCGGTCA
GATTTCTCCAATGAGGAGCGTTTCACAGGCTTTGATGCCCACTAC
ATGGCTGTGGATGTGGACGAGTGCAAGGAGAGGGAGGACGAGGAG
CTGTCCTGTGACCACTACTGCCACAACTACATTGGCGGCTACTAC
TGCTCCTGCCGCTTCGGCTACATCCTCCACACAGACAACAGGACC
TGCCGAGTGGAGTGCAGTGACAACCTCTTCACTCAAAGGACTGGG
GTGATCACCAGCCCTGACTTCCCAAACCCTTACCCCAAGAGCTCT
GAATGCCTGTATACCATCGAGCTGGAGGAGGGTTTCATGGTCAAC
CTGCAGTTTGAGGACATATTTGACATTGAGGACCATCCTGAGGTG
CCCTGCCCCTATGACTACATCAAGATCAAAGTTGGTCCAAAAGTT
TTGGGGCCTTTCTGTGGAGAGAAAGCCCCAGAACCCATCAGCACC
CAGAGCCACAGTGTCCTGATCCTGTTCCATAGTGACAACTCGGGA
GAGAACCGGGGCTGGAGGCTCTCATACAGGGCTGCAGGAAATGAG
TGCCCAGAGCTACAGCCTCCTGTCCATGGGAAAATCGAGCCCTCC
CAAGCCAAGTATTTCTTCAAAGACCAAGTGCTCGTCAGCTGTGAC
ACAGGCTACAAAGTGCTGAAGGATAATGTGGAGATGGACACATTC
CAGATTGAGTGTCTGAAGGATGGGACGTGGAGTAACAAGATTCCC
ACCTGTAAAAAAAATGAAATCGATCTGGAGAGCGAACTCAAGTCA
GAGCAAGTGACAGAGTGA
MAp44 CUB1 amino acid sequence
(SEQ ID NO: 46)
FGQIQSPGYPDSYPSDSEVTWNITVPDGFRIKLYFMHFNLESSYL
CEYDYVKVETEDQVLATFCGRETTDTEQTPGQEVVLSPGSFMSIT
FRSDFSNEERFTGFDAHYMA
MAp44 CUB1 nucleic acid sequence
(SEQ ID NO: 47)
TTTGGCCAGATCCAGTCGCCTGGTTATCCAGACTCCTATCCCAGT
GATTCAGAGGTGACTTGGAATATCACTGTCCCAGATGGGTTTCGG
ATCAAGCTTTACTTCATGCACTTCAACTTGGAATCCTCCTACCTT
TGTGAATATGACTATGTGAAGGTAGAAACTGAGGACCAGGTGCTG
GCAACCTTCTGTGGCAGGGAGACCACAGACACAGAGCAGACTCCC
GGCCAGGAGGTGGTCCTCTCCCCTGGCTCCTTCATGTCCATCACT
TTCCGGTCAGATTTCTCCAATGAGGAGCGTTTCACAGGCTTTGAT
GCCCACTACATGGCT
MAp44 EGF amino acid sequence
(SEQ ID NO: 48)
DVDECKEREDEELSCDHYCHNYIGGYYCSCRFGYILHT
MAp44 EGF nucleic acid sequence
(SEQ ID NO: 49)
GATGTGGACGAGTGCAAGGAGAGGGAGGACGAGGAGCTGTCCTGT
GACCACTACTGCCACAACTACATTGGCGGCTACTACTGCTCCTGC
CGCTTCGGCTACATCCTCCACACA
MAp44 CUB2 amino acid sequence
(SEQ ID NO: 50)
CSDNLFTQRTGVITSPDFPNPYPKSSECLYTIELEEGFMVNLQFE
DIFDIEDHPEVPCPYDYIKIKVGPKVLGPFCGEKAPEPISTQSHS
VLILFHSDNSGENRGWRLSYRA
MAp44 CUB2 nucleic acid sequence
(SEQ ID NO: 51)
TGCAGTGACAACCTCTTCACTCAAAGGACTGGGGTGATCACCAGC
CCTGACTTCCCAAACCCTTACCCCAAGAGCTCTGAATGCCTGTAT
ACCATCGAGCTGGAGGAGGGTTTCATGGTCAACCTGCAGTTTGAG
GACATATTTGACATTGAGGACCATCCTGAGGTGCCCTGCCCCTAT
GACTACATCAAGATCAAAGTTGGTCCAAAAGTTTTGGGGCCTTTC
TGTGGAGAGAAAGCCCCAGAACCCATCAGCACCCAGAGCCACAGT
GTCCTGATCCTGTTCCATAGTGACAACTCGGGAGAGAACCGGGGC
TGGAGGCTCTCATACAGGGCT
MAp44 CCP1 amino acid sequence
(SEQ ID NO: 52)
CPELQPPVHGKIEPSQAKYFFKDQVLVSCDTGYKVLKDNVEMDTF
QIECLKDGTWSNKIPTCK
MAp44 CCP1 nucleic acid sequence
(SEQ ID NO: 53)
TGCCCAGAGCTACAGCCTCCTGTCCATGGGAAAATCGAGCCCTCC
CAAGCCAAGTATTTCTTCAAAGACCAAGTGCTCGTCAGCTGTGAC
ACAGGCTACAAAGTGCTGAAGGATAATGTGGAGATGGACACATTC
CAGATTGAGTGTCTGAAGGATGGGACGTGGAGTAACAAGATTCCC
ACCTGTAAA
Signal peptide of the human CD5 protein
(SEQ ID NO: 54)
MPMGSLQPLATLYLLGMLVAS
Signal peptide of the human CR2 protein
(SEQ ID NO: 55)
MGAAGLLGVFLALVAPG
Signal peptide of the human CR2 protein
(SEQ ID NO: 56)
MGAAGLLGVFLALVAPGVLG
B4 scFV nucleic acid sequence
(SEQ ID NO: 57)
GACACGTGATCAGCCGCCACCATGCCCATGGGGTCTCTGCAACCG
CTGGCCACCTTGTACCTGCTGGGGATGCTGGTCGCTTCCGTGCTA
GCGCATCATCATCATCATCATGTGAAACTGCAGGAGTCAGGGGCT
GAGCTTGTGAAGCCTGGGGCTTCAGTGAAGCTGTCCTGCAAGGCT
TCTGGCTACACCTTCACCAGCTACTGGATGCACTGGGTGAAGCAG
AGGCCTGGACGAGGCCTTGAGTGGATTGGAAGGATTGGTCCTAAT
AGTGGTGGTACTAAGTACAATGAGAAGTTCAAGAGCAAGGCCACA
CTGACTGTAGACAAACCCTCCAGCACAGCCTACATGCAGCTCAGC
AGCCTGACATCTGAGGACTCTGCGGTCTATTATTGTGCAAGAAGA
ATGGTAAAGGGGTGCTATGGACTACTGGGGCCAAGGGACCACGGT
CACCGTCTCCTCAAAGGGCGAATTCCAGCACACTGGCGGCCGTTA
CTAGTGGATCCGAGCTCGGTACCAAGCTTGGCGTCAGGAGGCGGT
GGCGGCTCGGGTGGCGGCGGCTCTTGGATATCTGCAGAATTCGCC
CTTGACATTGAGCTCACCCAGTCTCCAACCACCATGGCTGCATCT
CCCGGGGAGAAGATCACTATCACCTGCAGTGCCAGCTCAAGTATA
AGTTCCAATTACTTGCATTGGTATCAGCAGAAGCCAGGATTCTCC
CCTAAACTCTTGATTTATAGGACATCCAATCTGGCTTCTGGAGTC
CCAGCTCGCTTCAGTGGCAGTGGGTCTGGGACCTCTTACTCTCTC
ACAATTGGCACCATGGAGGCTGAAGATGTTGCCACTTACTACTGC
CAGCAGGGTAGTAGTATACCACGTACACGTTCGGAGGGGGCACCA
AGCTGGAAATAATAGACTAGTCGTGCG
C2 scFV nucleic acid sequence
(SEQ ID NO: 58)
GACACGAAGCTTGCCGCCACCATGCCCATGGGGTCTCTGCAACCG
CTGGCCACCTTGTACCTGCTGGGGATGCTGGTCGCTTCCGTGCTA
GCGCATCATCATCATCATCATGTCAAGCTGCAGGAGTCTGGGACT
GAACTGGTGAAGCCTGGGGCTTCAGTGAAGCTGTCCTGCAAGGCT
TCTGGCTACACCTTCACCAGCTACTGGATGCACTGGGTGAAGCAG
AGGCCTGGACAAGGCCTTGAGTGGATTGGAAATATTAATCCTAGC
AATGGTGGTACTAACTACAATGAGAAGTTCAAGAGCAAGGCCACA
CTGACTGTAGACAAATCCTCCAGCACAGCCTACATGCAGCTCAGC
AGCCTGACATCTGAGGACTCTGCGGTCTATTATTGTGCAAGAAGA
GGCATACGGTTACGACACTTTGACTACTGGGGCCAAGGGACCACG
GTCACCGTCTCCTCAAGGGCGAATTCTGCAGATATCCATCACACT
GGCGGCCGCTCGAGCATGCATCTAGAGGGCCCAATTCGCCCTATA
GTGAGTCGTATATCAGGAGGCGGTGGCGGCTCGGGTGGCGGCGGC
TCTTGGATATCTGCAGAATTCGCCCTTGACATTGTGATGACACAG
TCTCCTGCTTCCTTAGCTGTATCTCTGGGGCAGAGGGCCACCATC
TCATACAGGGCCAGCAAAAGTGTCAGTACATCTGGCTATAGTTAT
ATGCACTGGAACCAACAGAAACCAGGACAGCCACCCAGACTCCTC
ATCTATCTTGTATCCAACCTAGAATCTGGGGTCCCTGCCAGGTTC
AGTGGCAGTGGGTCTGGGACAGACTTCACCCTCAACATCCATCCT
GTGGAGGAGGAGGATGCTGCAACCTATTACTGTCAGCACATTAGG
GAGCTTACACGTTCGGAGGGGGGACCAAGCTGGAAATAATAGCCC
GGGCGTGCG

1-56. (canceled)

57. A targeting construct comprising a targeting moiety and a MAp44 polypeptide or a fragment thereof, wherein:
(1) the MAp44 polypeptide or fragment thereof inhibits complement activation, and comprises
(a) amino acids 1-137, amino acids 1-176, amino acids 1-296, or amino acids 1-363 of SEQ ID NO: 44; or,
(b) a sequence selected from the group consisting of SEQ ID NOs: 46, 48, 50, and 52; and,
(2) the targeting moiety is an antibody or an antigen-binding fragment thereof that specifically binds to Annexin IV and comprises:
(i) a light chain variable domain comprising a light chain complementarity determining region (LC-CDR) 1 comprising the sequence of SEQ ID NO: 1 or 7, an LC-CDR2 comprising the sequence of SEQ ID NO: 2 or 8, and an LC-CDR3 comprising the sequence of SEQ ID NO: 3 or 9; and
(ii) heavy chain variable domain comprising a heavy chain complementarity determining region (HC-CDR) 1 comprising the sequence of SEQ ID NO: 4 or 10, an HC-CDR2 comprising the sequence of SEQ ID NO: 5 or 11, and an HCCDR3 comprising the sequence of SEQ ID NO: 6 or 12.

58. The targeting construct of claim 57, wherein the light chain variable domain comprises the sequence of SEQ ID NO: 13 or 14; wherein the heavy chain variable domain comprises the sequence of SEQ ID NO: 15 or 16; and/or wherein the antibody or antigen-binding fragment thereof is an scFv comprising the sequence of SEQ ID NO: 17 or 18.

59. The targeting construct of claim 58, wherein the antibody or antigen-binding fragment thereof competitively inhibits the binding of monoclonal antibody B4 to Annexin IV.

60. The targeting construct of claim 58, wherein the antibody or antigen-binding fragment thereof binds to the same epitope as monoclonal antibody B4.

60. A targeting construct comprising a targeting moiety, and a MAp44 polypeptide or a fragment thereof, wherein:
(1) the MAp44 polypeptide or fragment thereof inhibits complement activation, and comprises
(a) amino acids 1-137, amino acids 1-176, amino acids 1-296, or amino acids 1-363 of SEQ ID NO: 44; or,
(b) a sequence selected from the group consisting of SEQ ID NOs: 46, 48, 50, and 52; and,
(2) the targeting moiety is an antibody or an antigen-binding fragment thereof that specifically binds to a phospholipid and comprises:
(i) a light chain variable domain comprising an LC-CDR1 comprising the sequence of SEQ ID NO: 25 or 31, an LC-CDR2 comprising the sequence of SEQ ID NO: 26 or 32, and an LC-CDR3 comprising the sequence of SEQ ID NO: 27 or 33; and
(ii) a heavy chain variable domain comprising an HC-CDR1 comprising the sequence of SEQ ID NO: 28, an HC-CDR2 comprising the sequence of SEQ ID NO: 29, and an HC-CDR3 comprising the sequence of SEQ ID NO: 30.

61. The targeting construct of claim 60, wherein the light chain variable domain comprises the sequence of SEQ ID NO: 34 or 35; the heavy chain variable domain comprises the sequence of SEQ ID NO: 36; and/or the antibody or antigen-binding fragment is an scFv comprising the sequence of SEQ ID NO: 37 or 38.

62. The targeting construct of claim 60, wherein the antibody or antigen-binding fragment thereof competitively inhibits the binding of monoclonal antibody C2 to the phospholipid.

63. The targeting construct of claim 60, wherein the antibody or antigen-binding fragment thereof binds to the same epitope as monoclonal antibody C2.

64. The targeting construct of claim 60, wherein the phospholipid is selected from the group consisting of phosphatidylethanolamine (PE), cardiolipin (CL), and phosphatidylcholine (PC).

65. The targeting construct of claim 60, wherein the antibody or antigen binding fragment thereof binds to MDA.

66. The targeting construct of claim 57, wherein the construct is a fusion protein.

67. The targeting construct of claim 66, wherein the antibody or antigen-binding fragment thereof, and the MAp44 polypeptide or fragment thereof, are linked by a peptide linker.

68. The targeting construct of claim 57, wherein the antigen-binding fragment thereof is a scFv, Fab, Fab′, or F(ab′)2.

69. A pharmaceutical composition comprising the targeting construct of claim 57 and a pharmaceutically acceptable carrier.
